(19) United States
(12) Patent Application Publication
(10) Pub. No.: US 2026/0308158 A1
(43) Pub. Date: Oct. 8, 2026

(54) TARGETING RNAS ASSOCIATED WITH TAUOPATHIES WITH SMALL MOLECULES

(71) Applicant: University of Florida Research Foundation, Incorporated, Gainesville, FL (US)

(72) Inventor: Matthew D. Disney, Gainesville, FL (US)

(73) Assignee: University of Florida Research Foundation, Incorporated, Gainesville, FL (US)

(21) Appl. No.: 19/483,416

(22) PCT Filed: May 10, 2024
(86) PCT No.: PCT/US2024/028867

§ 371 Date: Nov. 11, 2025

Related U.S. Application Data
(60) Provisional application No. 63/502,051, filed on May 12, 2023.

(51) Int. Cl. C07D 487/04; A61K 31/381; A61K 31/4436; A61K 31/444; A61K 31/519; A61P 25/28; C07D 333/76; C07D 409/12; C07D 409/14

(52) U.S. Cl. CPC C07D 487/04; A61K 31/381; A61K 31/4436; A61K 31/444; A61K 31/519; A61P 25/28; C07D 333/76; C07D 409/12; C07D 409/14

(57) ABSTRACT

The present disclosure provides compounds of the formulae herein (e.g., Formula (I)), and pharmaceutically acceptable salts, solvates, hydrates, polymorphs, co-crystals, tautomers, stereoisomers, isotopically labeled compounds, and prodrugs thereof, which stabilize an RNA target. The present disclosure also provides pharmaceutical compositions and kits comprising the compounds, or pharmaceutically acceptable salts, solvates, hydrates, polymorphs, co-crystals, tautomers, stereoisomers, isotopically labeled compounds, or prodrugs thereof, and methods of treating or preventing diseases by administering to a subject in need thereof the compounds, or pharmaceutically acceptable salts, solvates, hydrates, polymorphs, co-crystals, tautomers, stereoisomers, isotopically labeled compounds, or prodrugs thereof, or pharmaceutical compositions thereof.

### CROSS-REFERENCE TO RELATED APPLICATIONS

This application claims the benefit of and priority under 35 U.S.C. § 119 (e) to U.S. Provisional Application No. 63/502,051, filed May 12, 2023, titled TARGETING RNAS ASSOCIATED WITH TAUOPATHIES WITH SMALL MOLECULES, the contents of which are incorporated herewith by reference in their entirety.

### GOVERNMENT SUPPORT

This invention was made with government support under Grant No. NS116846 awarded by the National Institutes of Health. The government has certain rights in the invention.

### REFERENCE TO AN ELECTRONIC SEQUENCE LISTING

The contents of the electronic sequence listing (U120270119WO00-SEQ-JDH.xml; Size: 39,704 bytes; and Date of Creation: May 9, 2024) is herein incorporated by reference in its entirety.

### BACKGROUND OF THE INVENTION

Over 90% of mRNAs are alternatively spliced, the outcome of which can be affected by a variety of factors, which include the sequence and structure of the pre-mRNA and the recognition thereof by trans-splicing factors such as the spliceosome and other proteins.¹⁻² Many diseases are caused by aberrant alternative splicing events including inherited genetic disease, cancer, and diseases of aging.³⁻⁴ The mechanistic underpinnings of disease-relevant alternative pre-mRNA splicing events can provide insights into the development of targeted therapeutics.

The aberrant alternative splicing of the microtubule-associated protein tau (MAPT) gene, which encodes the protein tau, causes the genetically defined disease, frontotemporal dementia and parkinsonism linked to chromosome 17 (FTDP-17).⁵ The gene comprises 16 exons, including alternatively spliced exons 2, 3, and 10, yielding six isoforms containing eight constitutive exons that affect the number of microtubule binding repeat (MTBR) domains at the carboxy-terminal.⁶ Inclusion or exclusion of exon 10 leads to three- or four-repeat MTBR isoforms (3R tau or 4R tau), respectively. In the healthy adult brain, 3R and 4R tau proteins are expressed equally,⁷ where 4R tau has additional phosphorylation sites that change protein solubility and ultimately lead to insoluble aggregates.⁸ In FTDP-17, a mutation in an RNA regulatory structure at the exon 10-intron junction dysregulates alternative splicing, tipping the balance of 3R: 4R towards the aggregation-prone 4R isoform.⁹ In addition, an increase in 4R tau without a genetic disposition has been shown to contribute to Alzheimer's disease (AD), as evidenced by aggregation of 4R in human brains.⁸ The contributions of 4R tau to disease are supported by the delivery of antisense oligonucleotides (ASOs) that switch splicing to increase 4R tau in a humanized tau (htau) mouse model, which causes behavioral defects observed in AD.¹⁰

Mechanistic studies used in conjunction with human genetics have deciphered the mechanism of FTDP-17. Various mutations, including a C-to-U intronic mutation (cytosine to uracil; C[+14]U; commonly referred to as disinhibition-dementia-parkinsonism-amyotrophy complex (DDPAC)), occur downstream of the 5′ splice site of tau exon 10 (FIG. 1A).¹¹ This mutation in the RNA's structure, as well as others, decreases the thermodynamic stability of the hairpin structure formed at the exon 10-intron 10 junction, a splicing regulatory element (SRE).¹² These mutations increase U1 small nuclear ribonuclear protein (snRNP) binding and hence exon 10 inclusion and 4R tau (FIG. 1A).¹² The proportion of 4R to 3R tau is therefore above a normal ratio in FTDP-17 patients (FIG. 1A). Conversely, the introduction of mutations that thermodynamically stabilize the SRE hairpin causes a reduction of the 4R/3R ratio,¹⁰ suggesting a potential strategy to reduce 4R tau in FTDP-17. That is, small molecules that bind to the RNA structure in MAPT pre-mRNA could limit U1 snRNP binding at the exon 10-intron junction and reduce the formation of toxic 4R tau protein.

Various small molecules have been discovered that bind to the SRE at the exon 10-intron junction, including the topoisomerase inhibitor mitoxantrone and aminoglycosides.¹³⁻¹⁴ Previously, a series of small molecules, including S1 (FIG. 6), that bind a well-folded adenine-containing bulge (A-bulge) region of the MAPT SRE and modulate splicing were identified from a database of experimentally derived RNA-small molecule interactions named Inforna.¹⁵ This method of lead identification searches for overlap between experimentally derived interactions and secondary structural elements present in cellular RNAs, outputting potential small molecule binders as well as associated fitness scores, metrics of selectivity and affinity. Using these small molecules as leads, a collection of compounds, including 1 (FIG. 6), was later identified that promoted exon skipping by directly targeting MAPT pre-mRNA in primary neuron cells harvested from humanized tau (htau) transgenic mice. Although the approach was promising in identifying drug-like chemical matter that bound the RNA, the IC₅₀ of 1 for directing MAPT exon 10 splicing was around 10 UM in cellular luciferase reporter assay, limiting its in vivo applicability.

### SUMMARY OF THE INVENTION

A general and pervasive challenge in the development of RNA-targeted small molecule medicines is their lead optimization, particularly for potency, selectively, and in vivo efficacy. One such strategy relies on targeting multiple sites within an RNA, which has been effective for RNA repeat expansions where small molecules selectively alleviate disease-associated phenotypes in patient-derived cells and mouse models.¹⁶⁻²⁰

In the present disclosure, a general strategy was developed to improve the potency and selectivity of small molecules that target non-repeating RNA targets utilizing MAPT pre-mRNA splicing as a proof-of-concept, while providing orally bioavailable compounds that cross the blood brain barrier (BBB). In particular, a base-triple-formation²¹ design strategy was leveraged around 1, affording 2, which selectively binds the MAPT SRE with sub-micromolar binding affinity (FIG. 1B). Further, 2 is at least 6-fold more potent in multiple biochemical assays and shows at least 10-fold improvement in a cellular luciferase reporter assay compared to 1. Target validation studies by Chemical Cross-linking and Isolation by Pull-down (Chem-CLIP)²²⁻²⁴ showed that 2 binds the SRE in cells, and RNA-seq analysis revealed that 2 is functionally selective transcriptome-wide. Furthermore, orally administrated 2 modulated Tau splicing in the brains of hTau mice and improved an associated aberrant behavioral phenotype. To the Applicant's knowledge, this is the first orally available bioactive small molecule to treat tauopathy in vivo.

Accordingly, in one aspect, the present disclosure provides compounds of Formula (I):

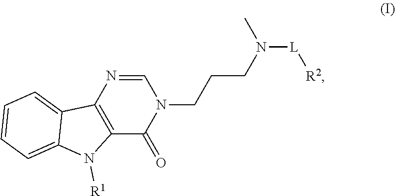

or pharmaceutically acceptable salts, solvates, hydrates, polymorphs, co-crystals, tautomers, stereoisomers, isotopically labeled compounds, or prodrugs thereof, wherein R¹, R², and L are as defined herein.

In another aspect, the present disclosure provides pharmaceutical compositions comprising a compound disclosed herein. In some embodiments, the pharmaceutical composition comprises an excipient.

In another aspect, the present disclosure provides methods of stabilizing an RNA target in a subject in need thereof or in a cell, tissue, or biological sample in a subject in need thereof or in a cell, tissue, or biological sample, comprising administering to the subject in need thereof or contacting the cell, tissue, or biological sample with an effective amount of a provided compound or pharmaceutical composition. In certain embodiments, the cell, tissue, or biological sample is in vivo. In certain embodiments, the cell, tissue, or biological sample is in vitro. In certain embodiments, the RNA target is microtubule-associated protein Tau (MAPT) pre-mRNA.

In another aspect, the present disclosure provides methods of decreasing a ratio of an amount of a first mRNA isoform to an amount of a second mRNA isoform in a subject in need thereof or in a cell, tissue, or biological sample in a subject in need thereof or in a cell, tissue, or biological sample, comprising administering to the subject in need thereof or contacting the cell, tissue, or biological sample with an effective amount of a provided compound or pharmaceutical composition. In certain embodiments, the cell, tissue, or biological sample is in vivo. In certain embodiments, the cell, tissue, or biological sample is in vitro. In certain embodiments, the method further comprises decreasing a ratio of an amount of a first protein isoform to an amount of a second protein isoform.

In another aspect, the present disclosure provides methods of treating or preventing a disease in a subject in need thereof, comprising administering to the subject in need thereof a provided compound or pharmaceutical composition. In certain embodiments, the disease is associated with microtubule-associated protein Tau (MAPT) pre-mRNA (e.g., a neurodegenerative disease (e.g., frontotemporal dementia and parkinsonism linked to chromosome 17 (FTDP-17), Alzheimer's disease)).

In another aspect, the present disclosure provides methods of preparing a compound of Formula (I-a-v):

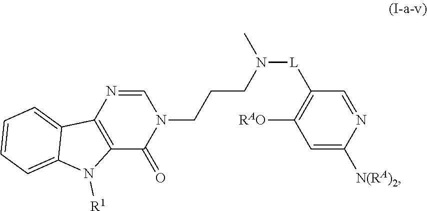

or a pharmaceutically acceptable salt, solvate, hydrate, polymorph, co-crystal, tautomer, stereoisomer, isotopically labeled compound, or prodrug thereof, wherein R¹, L, and R^{A} are as defined herein.

In another aspect, the present disclosure provides kits comprising a provided compound or pharmaceutical composition disclosed herein and instructions for its use.

It should be appreciated that the foregoing concepts, and the additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying drawings.

### BRIEF DESCRIPTION OF THE DRAWINGS

FIGS. 1A-1E show rational design of 2, which modulates alternative splicing of exon 10 by binding to an SRE present in MAPT pre-mRNA. FIG. 1A shows a schematic of alternative splicing of exons 9, 10, and 11 of MAPT pre-mRNA. U1 snRNP binds and unfolds the MAPT SRE at the exon 10-intron 10 junction, leading to exon 10 inclusion. DDPAC MAPT, a mutation associated with FTDP-17, has +14 C to U mutation that destabilizes the hairpin, promoting U1 snRNP binding and hence exon 10 inclusion, producing higher levels of 4R tau and an imbalance of the 4R/3R ratio. The binding of small molecules to the A-bulge site within SRE stabilizes the hairpin, increasing exon 10 skipping and production of 3R tau. FIG. 1B shows a structure of parent compound 1, which was previously identified.¹⁵ Compound 2 was rationally designed to form a base triple with a GC pair that closes the A-bulge, 1's binding site. FIG. 1C shows secondary structures of various MAPT SRE RNAs used in the present disclosure. The mini-gene luciferase reporters express luciferase in-frame with exon 10. The I17T mutants abolish the small molecule binding site by converting the A-bulge to an AU base pair. FIG. 1D shows HeLa cells transfected with a DDPAC or WT MAPT mini-gene reporter treated with 1 or 2, showing inhibition of luciferase activity in a dose dependent manner (n=8). FIG. 1E shows the effect of 1 and 2 on the alternative splicing of WT I17T or DDPAC I17T mutant luciferase reporters (n=4). Error bars indicate SD.

FIGS. 2A-2C show the structures of unbound and ligand-bound WT MAPT SRE. FIG. 2A shows previously reported structures of the bound WT stem mimic duplex.¹⁵ FIG. 2B shows the structure of the interactions formed between 1 and a duplex model of the WT MAPT SRE that was previously reported.¹⁵ FIG. 2C shows the Structure of the interactions formed between 2 and a duplex model of the WT MAPT SRE, as determined by NMR-restrained molecular dynamics (MD) simulations. For clarity just the triple base hydrogen bonds are shown.

FIGS. 3A-3H show target profiling in cells demonstrates direct target engagement of MAPT mRNA by 2 using Chemical Cross-Linking and Isolation by Pull-down (Chem-CLIP). FIG. 3A shows the effects of 1 and 2 on the ratio of 4R and 3R MAPT mRNA, as assessed by RT-qPCR in HeLa cells transfected with WT or DDPAC mini-genes, and in LAN5 neuroblastoma cells. Cells were treated with 10 μM of 1, 1.5 μM of 2, 0.5 μM of an ASO that direct tau splicing toward the 3R isoform, or 0.5 μM of a scrambled ASO control (n=3). FIG. 3B shows the effects of 1 and 2 on the ratio of 4R and 3R MAPT mRNA, as assessed by RT-qPCR in HeLa cells transfected with mini-genes expressing the mutants WT I17T or DDPAC I17T. Cells were treated with 10 μM of 1, 1.5 μM of 2, 0.5 μM of an ASO that direct tau splicing toward the 3R isoform, or 0.5 μM of a scrambled ASO control (n=3). FIG. 3C shows a scheme of Chem-CLIP to cross-link MAPT pre-mRNA to small molecule binders in cells to validate target engagement. FIG. 3D shows the compounds used in Chem-CLIP and competitive Chem-CLIP (C-Chem-CLIP) studies. Chem-CLIP probe 3 comprises the RNA-binding module 2, an alkyne purification module, and a cross-linking diazirine module. Control Chem-CLIP probe 4 lacks RNA-binding module 2. FIG. 3E shows pull-down of MAPT pre-mRNA by 3 or 4 in HeLa cells transfected with WT, DDPAC, WT I17T, or DDPAC I17T mini-genes (n=4). Pre-incubation of 2 diminished pull-down of the MAPT pre-mRNA by 3 in C-Chem-CLIP studies. FIG. 3F shows target validation studies in LAN5 cells completed by Chem-CLIP using Chem-CLIP probe 3 and control probe 4 (n=4). C-Chem-CLIP studies between 3 and increasing concentrations of 1 or 2, which were preincubated with LAN5 cells prior to addition of 3. FIG. 3G shows the effect of 2 (1.5 μM) on MAPT exon 10 alternative splicing in LAN5 cells at the protein level, as determined by western blotting (n=3). FIG. 3H shows the effects of 1, 2, and an exon 10 modulating ASO, or a scrambled ASO on MAPT exon 10 splicing outcomes in primary neurons harvested from htau mouse pups, as reported by the 4R/3R ratio (n=4). * P<0.05, ** P<0.01, and *** P<0.001 as determined by one-way ANOVA. Error bars indicate SD.

FIGS. 4A-4D show compound 2 mitigates disease-associated pathologies and rescues aberrant behavioral phenotypes in htau mice when orally administrated at 100 mg/kg q.o.d. FIG. 4A shows the effect of 2 on the MAPT 4R/3R ratio (top) or PSI₁₀ (PSI of MAPT exon 10, bottom) in htau mice, as compared to vehicle-treated mice (measured by RT-qPCR; n=6). FIG. 4B shows: Top, representative histological images of cortex from htau mice treated with vehicle or 2 and quantification thereof. Immunohistochemical staining was used to measure levels of phosphorylated tau (AT8 antibody; maker of tauopathy) and NeuN (marker of neuron viability). Bottom, quantification of AT8 positive area and NeuN positive neurons. FIG. 4C shows representative images of nests built by htau mice. Nesting activity was assessed using scoring criteria to rate the quality of nest construction and the amount of torn nestlet material on a scale of 1-5 as previously described.³⁷ FIG. 4D shows nesting scores upon treatment of WT or htau mice treated with vehicle or 2 (n=6). * P<0.05, ** P<0.01, *** P<0.001, as determined by one-way ANOVA. Error bars indicate SD.

FIGS. 5A-5B show a comparison of Risdiplam and small molecule 2 of the present disclosure. FIG. 5A shows FDA-approved small molecule Risdiplam modulates alternative splicing of the SMN2 pre-mRNA by targeting RNA-protein complex as a treatment for SMA. FIG. 5B shows small molecule 2 in the present disclosure modulates alternative splicing of tau pre-mRNA by directly targeting its RNA structural motif and closing base pair.

FIG. 6 shows structures of S1, 1, and the eight 2-aminopyridine analogs that were screened in cell-based luciferase assay. S1 and 1 were previously described and designed by an iterative process of file mining, in vitro and cellular assays, and structure-based design.¹⁸ M1 was not pursued for derivatization since M1 has a low calculated binding energy (−6.39 kcal/mol, Table 1) due to its weak interactions (solely hydrogen bonds) with the A-bulge binding pocket.¹⁸

FIG. 7 shows the interaction network of first five structures, those with the lowest free energies, extracted from the NMR ensemble of the RNA-S1 complex (PDB: 6VA2¹⁸). Stacking interactions with the neighboring GC base pair of the A-bulge (solid line) as well as hydrogen bonds (dashed lines) stabilize the bound state of S1. The RNA backbone is shown in cartoon and base pairs in stick representation. S1 is shown in stick representation.

FIG. 8 shows the interaction network of first five structures, those with the lowest free energies, extracted from the NMR ensemble of the RNA-M1 complex (PDB: 6VA318). Hydrogen bonds with the neighboring GC base pair of the A-bulge (dashed lines) as well as with backbone stabilize the bound state of M1. The RNA backbone is shown in cartoon and base pairs in stick representation. M1 is shown in stick representation.

FIG. 9 shows the interaction network of first five structures, those with the lowest free energies extracted from the NMR ensemble of the RNA-1 complex (PDB: 6VA418). Stacking interactions with the neighboring GC base pair of the A-bulge (solid lines) as well as hydrogen bonds (dashed lines) stabilize the bound state of 1. The RNA backbone is shown in cartoon and base pairs in stick representation. 1 is shown in stick representation.

FIGS. 10A-10B show the effect of derivatives on expression of luciferase in cell-based luciferase assays. FIG. 10A shows a schematic diagram of a minigene used in cell-based luciferase assays with HeLa cells transfected with a DDPAC or WT construct. The RNA hairpin at the exon 10-intron junction controls alternative splicing of U1 snRNP. The C(+14) U mutation destabilizes the hairpin, promoting U1 snRNP loading and exon 10 inclusion. When the small molecule binds to the A-bulge site, it stabilizes the hairpin and prevents RNA unfolding. The firefly luciferase reporter gene is in frame with exon 10. Luciferase is expressed when exon 10 is included. FIG. 10B shows results of luciferase reporter assay that reports on exon 10 inclusion in DDPAC mini-gene-transfected HeLa cells treated with compounds (n=4). The DDPAC mini-gene reporter was previously described.⁴⁴ Firefly luciferase is in-frame with exon 10. Thus, compounds that direct alternative splicing such that exon 10 is excluded reduce firefly luciferase activity. Note that 1 was tested at concentrations of 0.5, 1.5, 5, and 15 μM while its 2-aminopyridine conjugates were evaluated at 0.5, 1.5, and 5 μM; S1 and its 2-aminopyridine conjugates were treated at 0.1, 0.5, and 2.5 μM; * P<0.05, ** P<0.01, *** P<0.001, as determined by one-way ANOVA.

FIG. 11 shows the effect of 2 on the viability of (untransfected) HeLa cells, assessed by WST-1 cell proliferation reagent (n=4). ** P<0.01, as determined by one-way ANOVA.

FIGS. 12A-12C show percent spliced in index (PSI) values of MAPT exon 10 are downregulated by compounds. PSI₁₀ (PSI of MAPT exon 10) was downregulated by for 1 and for 2 compared to vehicle in LAN5 cells (FIG. 12A), in HeLa cells transfected by WT and DDPAC mini-gene (FIG. 12A) but not WT I17T or DDPAC I17T mutant (FIG. 12B) as well as in primary neurons harvested from htau mouse pups (FIG. 12C). PSI₁₀ of tau ASO but not scrambled ASO was also downregulated in all cell lines.

FIGS. 13A-13B show in vitro characterization of compounds, indicating that 2 binds to Tau SRE RNA with greater selectivity and affinity than 1. FIG. 13A shows binding curves of 1 or 2 and WT, WT I17T, DDPAC, and DDPAC I17T RNA, where the intrinsic fluorescence of the small molecule was measured as a function of RNA concentration (n=3). No saturable binding was observed with the addition of up to 50 μM of the I17T mutant RNAs. FIG. 13B shows binding curves of fluorescent binding assay between 1 or 2 and 7-deaza-guanosine WT RNA (n=3).

FIGS. 14A-14C show in vitro Chem-CLIP experiments demonstrating direct target engagement of Tau SRE RNA by 2. FIG. 14A shows the percentage of WT and WT+I17T mutant RNA pulled down in Chem-CLIP experiments as function of probe concentration, whether 3 and 4 (n=3). FIG. 14B shows the percentage of DDPAC and DDPAC+I17T mutant RNA pulled down in Chem-CLIP experiments as function of probe concentration, whether 3 and 4 (n=3). FIG. 14C shows results of in vitro C-Chem-CLIP experiments with WT and DDPAC RNAs and 2 and 3 (n=3). Increasing concentrations of 2 inhibits pulldown of RNAs by Chem-CLIP probe 3 in a dose-dependent manner. * P<0.05, ** P<0.01, and *** P<0.001 as determined by one-way ANOVA.

FIGS. 15A-15C show a biochemical assay to assess the ability of 1 and 2 to prevent binding of U1 snRNA, which facilitates inclusion of exon 10. FIG. 15A shows a schematic of FRET-based assay to assess inhibition of U1 snRNA binding of the tau SRE. FIG. 15B shows the effect of 1 and 2 (10 μM) on the ability of U1 snRNA to unfold a dually labeled tau RNA hairpin as a function of time (n=4). FIG. 15C shows fold-change in fluorescence intensity measured at t=10 min (end-point measurement) when tau SRE was incubated with varying concentrations of 1 and 2 (n=4). Compound 2 has around 5-fold lower IC₅₀ due to its higher binding affinity. Compound 2 was not tested at 200 μM due to insolubility.

FIGS. 16A-16B show the effects of 1 and 2 on the thermodynamic stability of tau RNA constructs. FIG. 16A shows the melting temperature (Tₘ) of WT, DDPAC, and DDPAC I17T RNAS upon addition of 1 and 2 (n=4). FIG. 16B shows the change in Tₘ of WT, DDPAC, and DDPAC I17T RNAs upon addition of 1 and 2 (n=4). ** P<0.01, and **** P<0.0001, as determined by two-tailed Student t-test.

FIGS. 17A-17D show 1D NMR spectral analysis of 2 and WT Tau Duplex. FIG. 17A shows the secondary structure of the tau RNA duplex construct used for NMR studies. FIG. 17B shows a 1D ¹H NMR spectrum of 2 (300 μM) at 298 K focusing on the aromatic region. FIG. 17C shows a WaterLOGSY NMR spectrum for 2 alone (300 μM), acquired in 95% H₂O and 5% D₂O at 298 K (top) and for 2 (300 μM) and the WT tau duplex (10 μM), affording a compound: RNA ratio 20:1, showing the same aromatic region (bottom). FIG. 17D shows a 1D ¹H NMR spectrum of exchangeable imino protons in the WT tau duplex (50 μM), acquired at 10° C. in the presence and absence of varying equivalents of 2. The imino proton spectrum in the absence of 2 shows base pairing of the RNA duplex in black and allows the assignment of the bases within the RNA duplex.

FIG. 18 shows the 2D NMR spectral analysis of 2 and WT tau Duplex. 2D ¹H-¹H D₂O NOESY NMR spectrum of the tau duplex RNA and 2, focusing on the non-exchangeable aromatic-sugar region. NOESY walks are highlighted with lines, with distinct duplex strands shown, AH2s highlighted with dashed-lines, and intramolecular NOEs shown on the far right (2-G17H1′).

FIGS. 19A-19A show Chem-CLIP-Map-Seq precisely maps the photo-cross-linking site of 3 in cells. FIG. 19A shows a scheme of the approach to identify the binding site of the Chem-CLIP probe 3 via photo-cross-linking. FIG. 19B shows representative Sanger sequencing results to identify the nucleotide cross-linked to 3, indicated with an arrow; that is, where reverse transcription is halted (left). Secondary structure of the SRE annotated with the 2 binding site, including the G that participates in a base triple interaction and the nucleotide where cross-linking occurs and the corresponding RT stop (right).

FIGS. 20A-20B show topology of the binding pocket induced by 2 (FIG. 20A) and 1 (FIG. 20B). The binding pocket provided by the flipped-out A-bulge adopts a rod-shaped topology induced by 2 (FIG. 20A) and sphere-like induced by 1 (FIG. 20B).

FIGS. 21A-21C show the effect of 2 and an ASO directing MAPT splicing towards the 3R isoform transcriptome-wide in LAN5 cells. FIG. 21A shows a Volcano plot showing transcriptome-wide changes induced by 2 (left, 1.5 μM) as compared to vehicle (0.1% (v/v) DMSO) and tau ASO (right, 0.5 μM) as compared to the same concentration of a scrambled ASO control (n=6). Dotted lines represent absolute fold change>2 and a P value<0.05. FIG. 21B shows the fold change of percent spliced in index (PSI) of MAPT exons. Left: evaluation of alternative splicing of MAPT exons upon treatment of LAN5 cells with 1.5 μM of 2. Right: evaluation of alternative splicing of MAPT exons upon treatment of LAN5 cells with 0.5 μM ASO. Changes shown are represented as log₂ fold increase (up) or decrease (down) in exon PSI relative to vehicle or scrambled ASO. Dotted lines represent absolute fold change>2. FIG. 21C shows plots of PSI of MAPT exons in FIG. 21B. * P<0.05 and ** P<0.01 as determined by two-tailed Student t-test.

FIGS. 22A-22B show Drug Metabolism and Pharmacokinetics (DMPK) analysis of 2 in C57BL/6 mice and the effect of 2 on the weight of WT and htau mice upon treatment. FIG. 22A shows plasma and brain exposure after oral administration of 2 (100 mg/kg) determined 48 h post-treatment (n=3). FIG. 22B shows WT mice or htau mice treated with 2 over 3 weeks have similar weights as vehicle-treated mice (n=7). Mice were orally administered (P.O.) with 2 (100 mg/kg) q.o.d . . .

FIG. 23 shows the effect of 2 on total MAPT levels in htau mice treated with vehicle or 2 (n=6). Note 2 reduced the 4R/3R MAPT ratio in hTau mice (FIG. 4A).

FIG. 24 shows shape calculations of compounds found in DrugBank⁴⁵ using the normalized principles moment of inertia (NPR1, NPR2). Based on the NPR values compounds shape is categorized as rod-, disc- or sphere-like.

FIG. 25 shows a schematic of how primary neuron cells were extracted and cultured from hTau mice brains as described in hTau mice primary neuron experiments. Total RNA was extracted after 48 h treatment of DMSO vehicle, 1, 2, Tau ASO and Scrambled ASO control. RT-qPCR was performed to measure 4R/3R ratio which calculated as 4R Tau mRNA expression divided by 3R Tau mRNA expression.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7th Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers. The term “isomers” is intended to include diastereoisomers, enantiomers, regioisomers, structural isomers, rotational isomers, tautomers, and the like. All such isomers of such compounds herein are expressly included in the present invention.

When a range of values (“range”) is listed, it encompasses each value and sub-range within the range. A range is inclusive of the values at the two ends of the range unless otherwise provided. For example, “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C12), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 2 to 20 carbon atoms and one or more carbon-carbon double bonds (e.g., 1, 2, 3, or 4 double bonds). In some embodiments, an alkenyl group has 2 to 20 carbon atoms (“C₂₋₂₀ alkenyl”). In some embodiments, an alkenyl group has 2 to 12 carbon atoms (“C₂₋₁₂ alkenyl”). In some embodiments, an alkenyl group has 2 to 11 carbon atoms (“C₂₋₁₁ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₂₀ alkenyl. In certain embodiments, the alkenyl group is a substituted C₂₋₂₀ alkenyl. In an alkenyl group, a C═C double bond for which the stereochemistry is not specified (e.g., —CH═CHCH₃ or

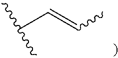

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 2 to 20 carbon atoms, at least one double bond, and 1 or more heteroatoms within the parent chain (“heteroC₂₋₂₀ alkenyl”). In certain embodiments, a heteroalkenyl group refers to a group having from 2 to 12 carbon atoms, at least one double bond, and 1 or more heteroatoms within the parent chain (“heteroC₂₋₁₂ alkenyl”). In certain embodiments, a heteroalkenyl group refers to a group having from 2 to 11 carbon atoms, at least one double bond, and 1 or more heteroatoms within the parent chain (“heteroC₂₋₁₁ alkenyl”). In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 carbon atoms, at least one double bond, and 1 heteroatom within the parent chain (“heteroC₂ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₂₀ alkenyl. In certain embodiments, the heteroalkenyl group is a substituted heteroC₂₋₂₀ alkenyl.

The term “alkynyl” refers to a radical of a straight-chain or branched hydrocarbon group having from 2 to 20 carbon atoms and one or more carbon-carbon triple bonds (e.g., 1, 2, 3, or 4 triple bonds) (“C₁₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₂₀ alkynyl. In certain embodiments, the alkynyl group is a substituted C₂₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 2 to 20 carbon atoms, at least one triple bond, and 1 or more heteroatoms within the parent chain (“heteroC₂₋₂₀ alkynyl”). In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 carbon atoms, at least one triple bond, and 1 heteroatom within the parent chain (“heteroC₂ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₂₀ alkynyl. In certain embodiments, the heteroalkynyl group is a substituted heteroC₂₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is optionally substituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo [3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is optionally substituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is optionally substituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being optionally substituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR)₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

wherein:
each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
each instance of R^{ee} is, independently, selected from C₁₋₁₀ alkyl, C₁₋₁₀ perhaloalkyl, C₁₋₁₀ alkenyl, C₃₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R&8 groups;
each instance of R⁸⁸ is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl) (C₁₋₆ alkyl), —N(OH) (C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl) C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R&8 substituents can be joined to form ═O or ═S; and each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb})) 2, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S) OR^{aa}, —SC(═S)N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group-NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; optionally substituted hydroxyl; optionally substituted thiol; optionally substituted amino; optionally substituted acyl, cyclic or acyclic, optionally substituted, branched or unbranched aliphatic; cyclic or acyclic, optionally substituted, branched or unbranched heteroaliphatic; cyclic or acyclic, optionally substituted, branched or unbranched alkyl; cyclic or acyclic, optionally substituted, branched or unbranched alkenyl; optionally substituted alkynyl; optionally substituted aryl, optionally substituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “phosphino” refers to the group —P(R^{cc})₂, wherein R^{cc} is as defined herein.

The term “phosphono” refers to the group —(P═O)(OR)₂, wherein R^{aa} and R^{cc} are as defined herein.

The term “phosphoramido” refers to the group —O(P═O)(N(R^{bb})₂)₂, wherein each R^{bb} is as defined herein.

The term “oxo” refers to the group ═O, and the term “thiooxo” refers to the group ═S.

Nitrogen atoms can be optionally substituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂ORE, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3rd edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino) acetamide, 3-(p-hydroxyphenyl) propanamide, 3-(o-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that includes the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten) acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR)₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri (p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3″′-[N-(imidazolylmethyl)]trityl Ether (IDTr-OR), 4,4′-Dimethoxy-3″′-[N-(imidazolylethyl) carbamoyl]trityl Ether (IETr-OR), 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy) ethyl carbonate (MTMEC-OR), 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(Rₐₐ)₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb}) 2)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O) 2 (CF₂) 3CF₃ (nonaflate, —ONf), or trifluoromethanesulfonate (triflate, —OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and Claims. The invention is not limited in any manner by the above exemplary listing of substituents.

As used herein, the term “salt” refers to any and all salts and encompasses pharmaceutically acceptable salts. The term “salt” refers to ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “co-crystal” refers to a crystalline structure comprising at least two different components (e.g., a compound and an acid), wherein each of the components is independently an atom, ion, or molecule. In certain embodiments, none of the components is a solvent. In certain embodiments, at least one of the components is a solvent. A co-crystal of a compound and an acid is different from a salt formed from a compound and the acid. In the salt, a compound is complexed with the acid in a way that proton transfer (e.g., a complete proton transfer) from the acid to a compound easily occurs at room temperature. In the co-crystal, however, a compound is complexed with the acid in a way that proton transfer from the acid to a herein does not easily occur at room temperature. In certain embodiments, in the co-crystal, there is substantially no proton transfer from the acid to a compound. In certain embodiments, in the co-crystal, there is partial proton transfer from the acid to a compound. Co-crystals may be useful to improve the properties (e.g., solubility, stability, and ease of formulation) of a compound.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

Compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers,” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “isotopically labeled compound” refers to a derivative of a compound that only structurally differs from the compound in that at least one atom of the derivative includes at least one isotope enriched above (e.g., enriched 3-, 10-, 30-, 100-, 300-, 1,000-, 3,000- or 10,000-fold above) its natural abundance, whereas each atom of the compound includes isotopes at their natural abundances. In certain embodiments, the isotope enriched above its natural abundance is ²H. In certain embodiments, the isotope enriched above its natural abundance is ¹³C, ¹⁵N, or ¹⁸O.

The term “prodrugs” refers to compounds that have cleavable groups and become by solvolysis or under physiological conditions the compounds described herein, which are pharmaceutically active in vivo. Such examples include choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgaard, H., Design of Prodrugs, pp. 7-9, 21-24, Elsevier, Amsterdam 1985). Prodrugs include, for example, esters prepared by reaction of the parent acid with a suitable alcohol, or amides prepared by reaction of the parent acid compound with a optionally substituted amine, or acid anhydrides, or mixed anhydrides.

The terms “pharmaceutical composition,” “composition,” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a pharmaceutical composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population. In some embodiments, the subject is at risk of developing a disease or condition due to environmental factors (e.g., exposure to the sun).

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for treating a disease or disorder associated with an RNA target in a subject in need thereof. In certain embodiments, a therapeutically effective amount is an amount sufficient for treating a disease or disorder associated with microtubule-associated protein Tau (MAPT) pre-mRNA (e.g., a neurodegenerative disease (e.g., frontotemporal dementia and parkinsonism linked to chromosome 17 (FTDP-17), Alzheimer's disease)) in a subject in need thereof. In certain embodiments, a therapeutically effective amount is an amount effective for treating a disease or disorder associated with microtubule-associated protein Tau (MAPT) pre-mRNA (e.g., a neurodegenerative disease (e.g., frontotemporal dementia and parkinsonism linked to chromosome 17 (FTDP-17), Alzheimer's disease)) in a subject in need thereof. In certain embodiments, a therapeutically effective amount is an amount effective for stabilizing microtubule-associated protein Tau (MAPT) pre-mRNA in a subject in need thereof or in a cell, tissue, or biological sample (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a therapeutically effective amount is an amount effective for decreasing a ratio of an amount of a first mRNA isoform to an amount of a second mRNA isoform (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%).

A “prophylactically effective amount” of a compound is an amount sufficient to prevent a condition, or one or more signs and/or symptoms associated with the condition or prevent its recurrence. In certain embodiments, the prophylactically effective amount is an amount that improves overall prophylaxis and/or enhances the prophylactic efficacy of another prophylactic agent. In certain embodiments, a prophylactically effective amount is an amount effective for preventing a disease or disorder associated with an RNA target in a subject in need thereof. In certain embodiments, a prophylactically effective amount is an amount effective for preventing a disease or disorder associated with microtubule-associated protein Tau (MAPT) pre-mRNA (e.g., a neurodegenerative disease (e.g., frontotemporal dementia and parkinsonism linked to chromosome 17 (FTDP-17), Alzheimer's disease)) in a subject in need thereof. In certain embodiments, a prophylactically effective amount is an amount effective for reducing the risk of developing a disease or disorder associated with microtubule-associated protein Tau (MAPT) pre-mRNA (e.g., a neurodegenerative disease (e.g., frontotemporal dementia and parkinsonism linked to chromosome 17 (FTDP-17), Alzheimer's disease)) in a subject in need thereof. In certain embodiments, a prophylactically effective amount is an amount effective for stabilizing microtubule-associated protein Tau (MAPT) pre-mRNA in a subject in need thereof or in a cell, tissue, or biological sample (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a prophylactically effective amount is an amount effective for decreasing a ratio of an amount of a first mRNA isoform to an amount of a second mRNA isoform (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%).

The term “gene” refers to a nucleic acid fragment that expresses a protein, including regulatory sequences preceding (5′ non-coding sequences) and following (3′ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonucleotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be introduced directly into the tissue site.

The polynucleotides may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, isotopes (e.g., radioactive isotopes), biotin, and the like.

“RNA transcript” refers to the product resulting from RNA polymerase-catalyzed transcription of a DNA sequence. When the RNA transcript is a complementary copy of the DNA sequence, it is referred to as the primary transcript, or it may be an RNA sequence derived from post-transcriptional processing of the primary transcript and is referred to as the mature RNA. “Messenger RNA (mRNA)” refers to the RNA that is without introns and can be translated into polypeptides by the cell. “cRNA” refers to complementary RNA, transcribed from a recombinant cDNA template. “cDNA” refers to DNA that is complementary to and derived from an mRNA template. The cDNA can be single-stranded or converted to double-stranded form using, for example, the Klenow fragment of DNA polymerase I.

A sequence “complementary” to a portion of an RNA, refers to a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

The terms “nucleic acid” or “nucleic acid sequence”, “nucleic acid molecule”, “nucleic acid fragment” or “polynucleotide” may be used interchangeably with “gene”, “mRNA encoded by a gene” and “cDNA”.

The term “mRNA” or “mRNA molecule” refers to messenger RNA, or the RNA that serves as a template for protein synthesis in a cell. The sequence of a strand of mRNA is based on the sequence of a complementary strand of DNA comprising a sequence coding for the protein to be synthesized.

The term “siRNA” or “siRNA molecule” refers to small inhibitory RNA duplexes that induce the RNA interference (RNAi) pathway, where the siRNA interferes with the expression of specific genes with a complementary nucleotide sequence. siRNA molecules can vary in length (e.g., between 18-30 or 20-25 basepairs, inclusive) and contain varying degrees of complementarity to their target mRNA in the antisense strand. Some siRNA have unpaired overhanging bases on the 5′ or 3′ end of the sense strand and/or the antisense strand. The term siRNA includes duplexes of two separate strands, as well as single strands that can form hairpin structures comprising a duplex region.

The term “microRNAs” or “miRNA” refers to small non-coding RNAs that are transcribed as primary transcripts that are processed first in the nucleus by a first nuclease to liberate the precursor miRNA, and then in the cytoplasm by a second nuclease to produce the mature miRNA. In certain embodiments, the term “microRNAs” or “miRNA” refers to small non-coding RNAs that are transcribed as primary transcripts that are processed first in the nucleus by Drosha to liberate the precursor miRNA, and then in the cytoplasm by Dicer to produce the mature miRNA.

The term “linker” refers to a bond or a divalent chemical moiety that is bonded to (i.e., that connects) two separate monovalent chemical moieties (e.g., the moieties N and R² in Formula (I)).

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Compounds

In one aspect, the present disclosure provides a compound of Formula (I):

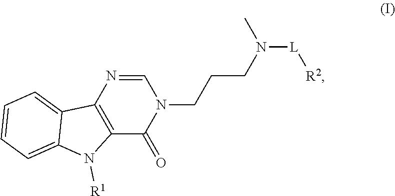

or a pharmaceutically acceptable salt, solvate, hydrate, polymorph, co-crystal, tautomer, stereoisomer, isotopically labeled compound, or prodrug thereof, wherein:
L is a linker;
R¹ is hydrogen or optionally substituted alkyl; and
R² is optionally substituted heteroaryl.

In certain embodiments, R¹ is hydrogen. In certain embodiments, R¹ is optionally substituted alkyl. In certain embodiments, R¹ is optionally substituted C₁₋₁₂ alkyl. In certain embodiments, R¹ is optionally substituted C₁₋₆ alkyl. In certain embodiments, R¹ is unsubstituted C₁₋₆ alkyl. In certain embodiments, R¹ is substituted C₁₋₆ alkyl. In certain embodiments, R¹ is optionally substituted methyl, optionally substituted ethyl, optionally substituted n-propyl, optionally substituted isopropyl, optionally substituted n-butyl, optionally substituted tert-butyl, optionally substituted sec-butyl, optionally substituted isobutyl, optionally substituted n-pentyl, optionally substituted 3-pentanyl, optionally substituted amyl, optionally substituted neopentyl, optionally substituted 3-methyl-2-butanyl, optionally substituted tert-amyl, or optionally substituted n-hexyl. In certain embodiments, R¹ is substituted methyl, substituted ethyl, substituted n-propyl, substituted isopropyl, substituted n-butyl, substituted tert-butyl, substituted sec-butyl, substituted isobutyl, substituted n-pentyl, substituted 3-pentanyl, substituted amyl, substituted neopentyl, substituted 3-methyl-2-butanyl, substituted tert-amyl, or substituted n-hexyl. In certain embodiments, R¹ is unsubstituted methyl, unsubstituted ethyl, unsubstituted n-propyl, unsubstituted isopropyl, unsubstituted n-butyl, unsubstituted tert-butyl, unsubstituted sec-butyl, unsubstituted isobutyl, unsubstituted n-pentyl, unsubstituted 3-pentanyl, unsubstituted amyl, unsubstituted neopentyl, unsubstituted 3-methyl-2-butanyl, unsubstituted tert-amyl, or unsubstituted n-hexyl. In certain embodiments, R¹ is —CH₃.

In certain embodiments, R² is optionally substituted heteroaryl. In certain embodiments, R² is optionally substituted 5-14 membered heteroaryl. In certain embodiments, R² is optionally substituted monocyclic heteroaryl. In certain embodiments, R² is optionally substituted 5- to 6-membered, monocyclic heteroaryl. In certain embodiments, R² is optionally substituted pyrrolyl, optionally substituted furanyl, optionally substituted thiophenyl, optionally substituted imidazolyl, optionally substituted pyrazolyl, optionally substituted oxazolyl, optionally substituted isoxazolyl, optionally substituted thiazolyl, optionally substituted isothiazolyl, optionally substituted triazolyl, optionally substituted oxadiazolyl, optionally substituted thiadiazolyl, or optionally substituted tetrazolyl. In certain embodiments, R² is optionally substituted pyridinyl, optionally substituted pyridazinyl, optionally substituted pyrimidinyl, optionally substituted pyrazinyl, optionally substituted triazinyl, optionally substituted tetrazinyl, optionally substituted oxepinyl, or optionally substituted thiepinyl. In certain embodiments, R² is optionally substituted bicyclic heteroaryl (e.g. optionally substituted bicyclic, 9- or 10-membered heteroaryl, wherein 1, 2, 3, or 4 atoms in the heteroaryl ring system are independently oxygen, nitrogen, or sulfur). In certain embodiments, R² is optionally substituted indolyl, optionally substituted isoindolyl, optionally substituted indazolyl, optionally substituted benzotriazolyl, optionally substituted benzothiophenyl, optionally substituted isobenzothiophenyl, optionally substituted benzofuranyl, optionally substituted benzoisofuranyl, optionally substituted benzimidazolyl, optionally substituted benzoxazolyl, optionally substituted benzisoxazolyl, optionally substituted benzoxadiazolyl, optionally substituted benzthiazolyl, optionally substituted benzisothiazolyl, optionally substituted benzthiadiazolyl, optionally substituted indolizinyl, optionally substituted purinyl. In certain embodiments, R² is optionally substituted naphthyridinyl, optionally substituted pteridinyl, optionally substituted quinolinyl, optionally substituted isoquinolinyl, optionally substituted cinnolinyl, optionally substituted quinoxalinyl, optionally substituted phthalazinyl, or optionally substituted quinazolinyl. In certain embodiments, R² is optionally substituted pyridinyl, optionally substituted pyrimidinyl, optionally substituted indolyl, optionally substituted oxazolyl, optionally substituted isoxazolyl, optionally substituted pyrrolyl, optionally substituted furanyl, optionally substituted pyrazolyl, optionally substituted imidazolyl, optionally substituted benzimidazolyl, or optionally substituted indazolyl. In certain embodiments, R² is heteroaryl substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A}) SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A}) SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A}) SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A}) 2 groups.

In certain embodiments, R² is optionally substituted pyridyl. In certain embodiments, R² is pyridyl substituted with one or more of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A}) SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A}) SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A}) SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A}) 2.

In certain embodiments, R² is

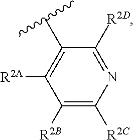

wherein:
each of R^{2A}, R^{2B}, R^{2C}, and R^{2D} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A}) SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; and
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom or atoms to form an optionally substituted heterocyclic ring or optionally substituted heteroaryl ring.

In certain embodiments, the compound of Formula (I) is of Formula (I-a-i):

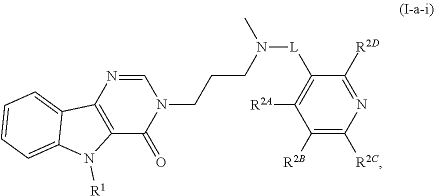

or a pharmaceutically acceptable salt, solvate, hydrate, polymorph, co-crystal, tautomer, stereoisomer, isotopically labeled compound, or prodrug thereof, wherein:
each of R^{2A}, R^{2B}, R^{2C}, and R^{2D} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; and
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom or atoms to form an optionally substituted heterocyclic ring or optionally substituted heteroaryl ring.

In certain embodiments, the compound of Formula (I-a-i) is of Formula (I-a-ii):

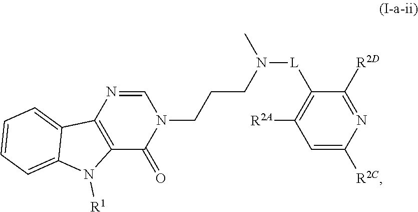

or a pharmaceutically acceptable salt, solvate, hydrate, polymorph, co-crystal, tautomer, stereoisomer, isotopically labeled compound, or prodrug thereof.

In certain embodiments, the compound of Formula (I-a-i) is of Formula (I-a-iii):

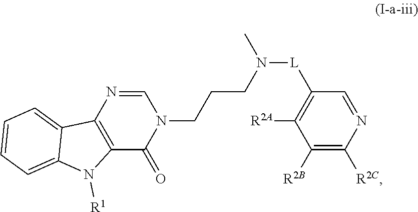

or a pharmaceutically acceptable salt, solvate, hydrate, polymorph, co-crystal, tautomer, stereoisomer, isotopically labeled compound, or prodrug thereof.

In certain embodiments, the compound of Formula (I-a-i) is of Formula (I-a-iv):

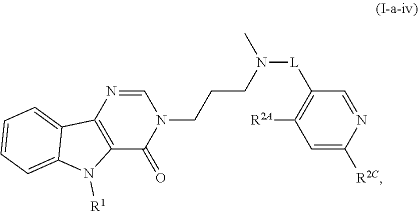

or a pharmaceutically acceptable salt, solvate, hydrate, polymorph, co-crystal, tautomer, stereoisomer, isotopically labeled compound, or prodrug thereof.

In certain embodiments, R^{2A} is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

In certain embodiments, R^{2B} is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

In certain embodiments, R^{2C} is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

In certain embodiments, R^{2D} is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

In certain embodiments, at least one of R^{2A}, R^{2B}, R^{2C}, and R^{2D} is hydrogen, —OR^{A}, or —N(R^{A})₂. In certain embodiments, each of R^{2A}, R^{2B}, R^{2C}, and R^{2D} is independently hydrogen, —OR^{A}, or —N(R^{A})₂. In certain embodiments, at least one of R^{2A}, R^{2B}, R^{2C}, and R^{2D} is hydrogen. In certain embodiments, R^{2A} is hydrogen. In certain embodiments, R^{2B} is hydrogen. In certain embodiments, R^{2C} is hydrogen. In certain embodiments, R^{2D} is hydrogen. In certain embodiments, R^{2B} is hydrogen and R^{2D} is hydrogen. In certain embodiments, R^{2A} is hydrogen, —OR^{A}, or —N(R^{A})₂, R^{2B} is hydrogen, R^{2C} is hydrogen, —OR^{A}, or —N(R^{A})₂, and R^{2D} is hydrogen. In certain embodiments, R^{2A} is —OR^{A} or —N(R^{A})₂, R^{2B} is hydrogen, R^{2C} is —OR^{A} or —N(R^{A})₂, and R^{2D} is hydrogen. In certain embodiments, R^{2A} is —OR^{A}, R^{2B} is hydrogen, R^{2C} is —OR^{A} or —N(R^{A})₂, and R^{2D} is hydrogen. In certain embodiments, R^{2A} is —OR^{A} or —N(R^{A})₂, R^{2B} is hydrogen, R^{2C} is —N(R^{A})₂, and R^{2D} is hydrogen. In certain embodiments, R^{2A} is —OR^{A}, R^{2B} is hydrogen, R^{2C} is —N(R^{A})₂, and R^{2D} is hydrogen.

In certain embodiments, at least one of R^{2A}, R^{2B}, R^{2C}, and R^{2D} is —OR^{A}. In certain embodiments, R^{2A} is —OR^{A}. In certain embodiments, R^{2B} is —OR^{A}. In certain embodiments, R^{2C} is —OR^{A}. In certain embodiments, R^{2D} is —OR^{A}. In certain embodiments, R^{2A} is —OR^{A}, R^{2B} is hydrogen, —OR^{A}, or —N(R^{A})₂, R^{2C} is hydrogen, —OR^{A}, or —N(R^{A})₂, and R^{2D} is hydrogen, —OR^{A}, or —N(R^{A})₂. In certain embodiments, R^{2A} is —OR^{A}, R^{2B} is hydrogen, R^{2C} is hydrogen, —OR^{A}, or —N(R^{A})₂, and R^{2D} is hydrogen, —OR^{A}, or —N(R^{A})₂. In certain embodiments, R^{2A} is —OR^{A}, R^{2B} is hydrogen, —OR^{A}, or —N(R^{A})₂, R^{2C} is —OR^{A} or —N(R^{A})₂, and R^{2D} is hydrogen, —OR^{A}, or —N(R^{A})₂. In certain embodiments, R^{2A} is —OR^{A}, R^{2B} is hydrogen, —OR^{A}, or —N(R^{A})₂, R^{2C} is —N(R^{A})₂, and R^{2D} is hydrogen, —OR^{A}, or —N(R^{A})₂. In certain embodiments, R^{2A} is —OR^{A}, R^{2B} is hydrogen, —OR^{A}, or —N(R^{A})₂, R^{2C} is hydrogen, —OR^{A}, or —N(R^{A})₂, and R^{2D} is hydrogen. In certain embodiments, R^{2A} is —OR^{A}, R^{2B} is hydrogen, R^{2C} is hydrogen, —OR^{A}, or —N(R^{A})₂, and R^{2D} is hydrogen. In certain embodiments, R^{2A} is —OR^{A}, R^{2B} is hydrogen, R^{2C} is —OR^{A} or —N(R^{A})₂, and R^{2D} is hydrogen.

In certain embodiments, at least one of R^{2A}, R^{2B}, R^{2C}, and R^{2D} is —N(R^{A})₂. In certain embodiments, R^{2A} is —N(R^{A})₂. In certain embodiments, R^{2B} is —N(R^{A})₂. In certain embodiments, R^{2C} is —N(R^{A})₂. In certain embodiments, R^{2D} is —N(R^{A})₂. In certain embodiments, R^{2A} is hydrogen, —OR^{A}, or —N(R^{A})₂, R^{2B} is hydrogen, —OR^{A}, or —N(R^{A})₂, R^{2C} is —N(R^{A})₂, and R^{2D} is hydrogen, —OR^{A}, or —N(R^{A})₂. In certain embodiments, R^{2A} is —OR^{A} or —N(R^{A})₂, R^{2B} is hydrogen, —OR^{A}, or —N(R^{A})₂, R^{2C} is —N(R^{A})₂, and R^{2D} is hydrogen, —OR^{A}, or —N(R^{A})₂. In certain embodiments, R^{2A} is —OR^{A}, R^{2B} is hydrogen, —OR^{A}, or —N(R^{A})₂, R^{2C} is —N(R^{A})₂, and R^{2D} is hydrogen, —OR^{A}, or —N(R^{A})₂. In certain embodiments, R^{2A} is hydrogen, —OR^{A}, or —N(R^{A})₂, R^{2B} is hydrogen, R^{2C} is —N(R^{A})₂, and R^{2D} is hydrogen, —OR^{A}, or —N(R^{A})₂. In certain embodiments, R^{2A} is hydrogen, —OR^{A}, or —N(R^{A})₂, R^{2B} is hydrogen, —OR^{A}, or —N(R^{A})₂, R^{2C} is —N(R^{A})₂, and R^{2D} is hydrogen. In certain embodiments, R^{2A} is hydrogen, —OR^{A}, or —N(R^{A})₂, R^{2B} is hydrogen, R^{2C} is —N(R^{A})₂, and R^{2D} is hydrogen. In certain embodiments, R^{2A} is —OR^{A} or —N(R^{A})₂, R^{2B} is hydrogen, R^{2C} is —N(R^{A})₂, and R^{2D} is hydrogen.

In certain embodiments, R^{2A} is —OR^{A}, and R^{A} is hydrogen or optionally substituted alkyl. In certain embodiments, R^{2A} is —OR^{A}, and R^{A} is hydrogen. In certain embodiments, R^{2A} is —OH. In certain embodiments, R^{2A} is —OR^{A}, and R^{A} is optionally substituted alkyl. In certain embodiments, R^{2A} is —O(optionally substituted alkyl). In certain embodiments, R^{2A} is —O(optionally substituted C₁₋₁₂ alkyl). In certain embodiments, R^{2A} is —O(optionally substituted C₁₋₆ alkyl). In certain embodiments, R^{2A} is —O(unsubstituted C₁₋₆ alkyl). In certain embodiments, R^{2A} is —O(substituted C₁₋₆ alkyl). In certain embodiments, R^{2A} is —O(optionally substituted methyl), —O(optionally substituted ethyl), —O(optionally substituted n-propyl), —O(optionally substituted isopropyl), —O(optionally substituted n-butyl), —O(optionally substituted tert-butyl), —O(optionally substituted sec-butyl), —O(optionally substituted isobutyl), —O(optionally substituted n-pentyl), —O(optionally substituted 3-pentanyl), —O(optionally substituted amyl), —O(optionally substituted neopentyl), —O(optionally substituted 3-methyl-2-butanyl), —O(optionally substituted tert-amyl), or —O(optionally substituted n-hexyl). In certain embodiments, R^{2A} is —O(substituted methyl), —O(substituted ethyl), —O(substituted n-propyl), —O(substituted isopropyl), —O(substituted n-butyl), —O(substituted tert-butyl), —O(substituted sec-butyl), —O(substituted isobutyl), —O(substituted n-pentyl), —O(substituted 3-pentanyl), —O(substituted amyl), —O(substituted neopentyl), —O(substituted 3-methyl-2-butanyl), —O(substituted tert-amyl), or —O(substituted n-hexyl). In certain embodiments, R^{2A} is —O(unsubstituted methyl), —O(unsubstituted ethyl), —O(unsubstituted n-propyl), —O(unsubstituted isopropyl), —O(unsubstituted n-butyl), —O(unsubstituted tert-butyl), —O(unsubstituted sec-butyl), —O(unsubstituted isobutyl), —O(unsubstituted n-pentyl), —O(unsubstituted 3-pentanyl), —O(unsubstituted amyl), —O(unsubstituted neopentyl), —O(unsubstituted 3-methyl-2-butanyl), —O(unsubstituted tert-amyl), or —O(unsubstituted n-hexyl). In certain embodiments, R^{2A} is —O(optionally substituted methyl), —O(optionally substituted ethyl), —O(optionally substituted n-propyl), —O(optionally substituted n-butyl), —O(optionally substituted n-pentyl), or —O(optionally substituted n-hexyl). In certain embodiments, R^{2A} is —O(substituted methyl), —O(substituted ethyl), —O(substituted n-propyl), —O(substituted n-butyl), —O(substituted n-pentyl), or —O(substituted n-hexyl). In certain embodiments, R^{2A} is —O(unsubstituted methyl), —O(unsubstituted ethyl), —O(unsubstituted n-propyl), —O(unsubstituted n-butyl), —O(unsubstituted n-pentyl), or —O(unsubstituted n-hexyl). In certain embodiments, R^{2A} is —O(optionally substituted methyl). In certain embodiments, R^{2A} is —O(substituted methyl). In certain embodiments, R^{2A} is —O(unsubstituted methyl). In certain embodiments, R^{2A} is —OCH₃.

In certain embodiments, R^{2C} is —N(R^{A})₂, and each occurrence of R^{A} is independently hydrogen or optionally substituted alkyl. In certain embodiments, R^{2C} is —N(R^{A})₂, and each occurrence of R^{A} is independently hydrogen. In certain embodiments, R^{2C} is —NH₂. In certain embodiments, R^{2C} is —NHRA, and R^{A} is optionally substituted alkyl. In certain embodiments, R^{2C} is —NH(optionally substituted alkyl). In certain embodiments, R^{2C} is —NH(optionally substituted C₁₋₁₂ alkyl). In certain embodiments, R^{2C} is —NH(optionally substituted C₁₋₆ alkyl). In certain embodiments, R^{2C} is —NH(unsubstituted C₁₋₆ alkyl). In certain embodiments, R^{2C} is —NH(substituted C₁₋₆ alkyl). In certain embodiments, R^{2C} is —NH(optionally substituted methyl), —NH(optionally substituted ethyl), —NH(optionally substituted n-propyl), —NH(optionally substituted isopropyl), —NH(optionally substituted n-butyl), —NH(optionally substituted tert-butyl), —NH(optionally substituted sec-butyl), —NH(optionally substituted isobutyl), —NH(optionally substituted n-pentyl), —NH(optionally substituted 3-pentanyl), —NH(optionally substituted amyl), —NH(optionally substituted neopentyl), —NH(optionally substituted 3-methyl-2-butanyl), —NH(optionally substituted tert-amyl), or —NH(optionally substituted n-hexyl). In certain embodiments, R^{2C} is —NH(substituted methyl), —NH(substituted ethyl), —NH(substituted n-propyl), —NH(substituted isopropyl), —NH(substituted n-butyl), —NH(substituted tert-butyl), —NH(substituted sec-butyl), —NH(substituted isobutyl), —NH(substituted n-pentyl), —NH(substituted 3-pentanyl), —NH(substituted amyl), —NH(substituted neopentyl), —NH(substituted 3-methyl-2-butanyl), —NH(substituted tert-amyl), or —NH(substituted n-hexyl). In certain embodiments, R^{2C} is —NH(unsubstituted methyl), —NH(unsubstituted ethyl), —NH(unsubstituted n-propyl), —NH(unsubstituted isopropyl), —NH(unsubstituted n-butyl), —NH(unsubstituted tert-butyl), —NH(unsubstituted sec-butyl), —NH(unsubstituted isobutyl), —NH(unsubstituted n-pentyl), —NH(unsubstituted 3-pentanyl), —NH(unsubstituted amyl), —NH(unsubstituted neopentyl), —NH(unsubstituted 3-methyl-2-butanyl), —NH(unsubstituted tert-amyl), or —NH(unsubstituted n-hexyl). In certain embodiments, R^{2C} is —NH(optionally substituted methyl), —NH(optionally substituted ethyl), —NH(optionally substituted n-propyl), —NH(optionally substituted n-butyl), —NH(optionally substituted n-pentyl), or —NH(optionally substituted n-hexyl). In certain embodiments, R^{2C} is —NH(substituted methyl), —NH(substituted ethyl), —NH(substituted n-propyl), —NH(substituted n-butyl), —NH(substituted n-pentyl), or —NH(substituted n-hexyl). In certain embodiments, R^{2C} is —NH(unsubstituted methyl), —NH(unsubstituted ethyl), —NH(unsubstituted n-propyl), —NH(unsubstituted n-butyl), —NH(unsubstituted n-pentyl), or —NH(unsubstituted n-hexyl). In certain embodiments, R^{2C} is —NH(optionally substituted methyl). In certain embodiments, R^{2C} is —NH(substituted methyl). In certain embodiments, R^{2C} is —NH(unsubstituted methyl). In certain embodiments, R^{2C} is —NHCH₃.

In certain embodiments, R^{2A} is —O(optionally substituted alkyl), and R^{2C} is —NH₂. In certain embodiments, R^{2A} is —O(optionally substituted alkyl), and R^{2C} is —NH(optionally substituted alkyl). In certain embodiments, R^{2A} is —O(optionally substituted alkyl), and R^{2C} is —NH(optionally substituted C₁₋₆ alkyl). In certain embodiments, R^{2A} is —O(optionally substituted alkyl), and R^{2C} is —NH(optionally substituted methyl). In certain embodiments, R^{2A} is —O(optionally substituted alkyl), and R^{2C} is —NHCH₃. In certain embodiments, R^{2A} is —O(optionally substituted alkyl), and R^{2C} is —NH₂ or —NHCH₃. In certain embodiments, R^{2A} is —O(optionally substituted C₁₋₆ alkyl), and R^{2C} is —NH₂. In certain embodiments, R^{2A} is —O(optionally substituted C₁₋₆ alkyl), and R^{2C} is —NH(optionally substituted alkyl). In certain embodiments, R^{2A} is —O(optionally substituted C₁₋₆ alkyl), and R^{2C} is —NH(optionally substituted C₁₋₆ alkyl). In certain embodiments, R^{2A} is —O(optionally substituted C₁₋₆ alkyl), and R^{2C} is —NH(optionally substituted methyl). In certain embodiments, R^{2A} is —O(optionally substituted C₁₋₆ alkyl), and R^{2C} is —NHCH₃. In certain embodiments, R^{2A} is —O(optionally substituted C₁₋₆ alkyl), and R^{2C} is —NH₂ or —NHCH₃. In certain embodiments, R^{2A} is —O(optionally substituted methyl), and R^{2C} is —NH₂. In certain embodiments, R^{2A} is —O(optionally substituted methyl), and R^{2C} is —NH(optionally substituted alkyl). In certain embodiments, R^{2A} is —O(optionally substituted methyl), and R^{2C} is —NH(optionally substituted C₁₋₆ alkyl). In certain embodiments, R^{2A} is —O(optionally substituted methyl), and R^{2C} is —NH(optionally substituted methyl). In certain embodiments, R^{2A} is —O(optionally substituted methyl), and R^{2C} is —NHCH₃. In certain embodiments, R^{2A} is —O(optionally substituted methyl), and R^{2C} is —NH₂ or —NHCH₃. In certain embodiments, R^{2A} is —OCH₃, and R^{2C} is —NH₂. In certain embodiments, R^{2A} is —OCH₃, and R^{2C} is —NH(optionally substituted alkyl). In certain embodiments, R^{2A} is —OCH₃, and R^{2C} is —NH(optionally substituted C₁₋₆ alkyl). In certain embodiments, R^{2A} is —OCH₃, and R^{2C} is —NH(optionally substituted methyl). In certain embodiments, R^{2A} is —OCH₃, and R^{2C} is —NHCH₃. In certain embodiments, R^{2A} is —OCH₃, and R^{2C} is —NH₂ or —NHCH₃.

In certain embodiments, R^{2A} is —O(optionally substituted alkyl), R^{2B} is hydrogen, R^{2C} is —NH₂, and R^{2D} is hydrogen. In certain embodiments, R^{2A} is —O(optionally substituted alkyl), R^{2B} is hydrogen, R^{2C} is —NH(optionally substituted alkyl), and R^{2D} is hydrogen. In certain embodiments, R^{2A} is —O(optionally substituted alkyl), R^{2B} is hydrogen, R^{2C} is —NH(optionally substituted C₁₋₆ alkyl), and R^{2D} is hydrogen. In certain embodiments, R^{2A} is —O(optionally substituted alkyl), R^{2B} is hydrogen, R^{2C} is —NH(optionally substituted methyl), and R^{2D} is hydrogen. In certain embodiments, R^{2A} is —O(optionally substituted alkyl), R^{2B} is hydrogen, R^{2C} is —NHCH₃, and R^{2D} is hydrogen. In certain embodiments, R^{2A} is —O(optionally substituted alkyl), R^{2B} is hydrogen, R^{2C} is —NH₂ or —NHCH₃, and R^{2D} is hydrogen. In certain embodiments, R^{2A} is —O(optionally substituted C₁₋₆ alkyl), R^{2B} is hydrogen, R^{2C} is —NH₂, and R^{2D} is hydrogen. In certain embodiments, R^{2A} is —O(optionally substituted C₁₋₆ alkyl), R^{2B} is hydrogen, R^{2C} is —NH(optionally substituted alkyl), and R^{2D} is hydrogen. In certain embodiments, R^{2A} is —O(optionally substituted C₁₋₆ alkyl), R^{2B} is hydrogen, R^{2C} is —NH(optionally substituted C₁₋₆ alkyl), and R^{2D} is hydrogen. In certain embodiments, R^{2A} is —O(optionally substituted C₁₋₆ alkyl), R^{2B} is hydrogen, R^{2C} is —NH(optionally substituted methyl), and R^{2D} is hydrogen. In certain embodiments, R^{2A} is —O(optionally substituted C₁₋₆ alkyl), R^{2B} is hydrogen, R^{2C} is —NHCH₃, and R^{2D} is hydrogen. In certain embodiments, R^{2A} is —O(optionally substituted C₁₋₆ alkyl), R^{2B} is hydrogen, R^{2C} is —NH₂ or —NHCH₃, and R^{2D} is hydrogen. In certain embodiments, R^{2A} is —O(optionally substituted methyl), R^{2B} is hydrogen, R^{2C} is —NH₂, and R^{2D} is hydrogen. In certain embodiments, R^{2A} is —O(optionally substituted methyl), R^{2B} is hydrogen, R^{2C} is —NH(optionally substituted alkyl), and R^{2D} is hydrogen. In certain embodiments, R^{2A} is —O(optionally substituted methyl), R^{2B} is hydrogen, R^{2C} is —NH(optionally substituted C₁₋₆ alkyl), and R^{2D} is hydrogen. In certain embodiments, R^{2A} is —O(optionally substituted methyl), R^{2B} is hydrogen, R^{2C} is —NH(optionally substituted methyl), and R^{2D} is hydrogen. In certain embodiments, R^{2A} is —O(optionally substituted methyl), R^{2B} is hydrogen, R^{2C} is —NHCH₃, and R^{2D} is hydrogen. In certain embodiments, R^{2A} is —O(optionally substituted methyl), R^{2B} is hydrogen, R^{2C} is —NH₂ or —NHCH₃, and R^{2D} is hydrogen. In certain embodiments, R^{2A} is —OCH₃, R^{2B} is hydrogen, R^{2C} is —NH₂, and R^{2D} is hydrogen. In certain embodiments, R^{2A} is —OCH₃, R^{2B} is hydrogen, R^{2C} is —NH(optionally substituted alkyl), and R^{2D} is hydrogen. In certain embodiments, R^{2A} is —OCH₃, R^{2B} is hydrogen, R^{2C} is —NH(optionally substituted C₁₋₆ alkyl), and R^{2D} is hydrogen. In certain embodiments, R^{2A} is —OCH₃, R^{2B} is hydrogen, R^{2C} is —NH(optionally substituted methyl), and R^{2D} is hydrogen. In certain embodiments, R^{2A} is —OCH₃, R^{2B} is hydrogen, R^{2C} is —NHCH₃, and R^{2D} is hydrogen. In certain embodiments, R^{2A} is —OCH₃, R^{2B} is hydrogen, R^{2C} is —NH₂ or —NHCH₃, and R^{2D} is hydrogen.

In certain embodiments, the compound of Formula (I-a-i) is of Formula (I-a-v):

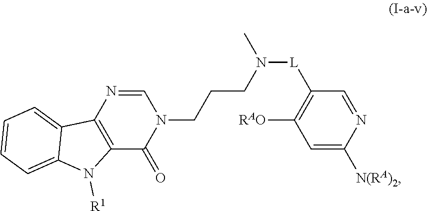

or a pharmaceutically acceptable salt, solvate, hydrate, polymorph, co-crystal, tautomer, stereoisomer, isotopically labeled compound, or prodrug thereof.

In certain embodiments, the compound of Formula (I-a-i) is of Formula (I-a-vi):

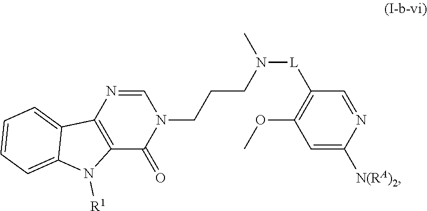

or a pharmaceutically acceptable salt, solvate, hydrate, polymorph, co-crystal, tautomer, stereoisomer, isotopically labeled compound, or prodrug thereof.

In certain embodiments, the compound of Formula (I-a-i) is of Formula (I-a-vii):

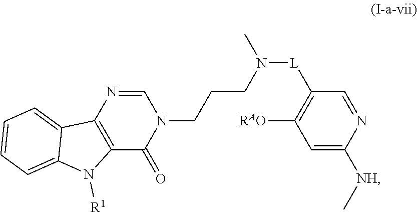

or a pharmaceutically acceptable salt, solvate, hydrate, polymorph, co-crystal, tautomer, stereoisomer, isotopically labeled compound, or prodrug thereof.

In certain embodiments, L is a linker. In certain embodiments, L is a bond, optionally substituted alkylene, optionally substituted alkenylene, optionally substituted alkynylene, optionally substituted heteroalkylene, optionally substituted heteroalkenylene, optionally substituted heteroalkynylene, optionally substituted heterocyclylene, optionally substituted carbocyclylene, optionally substituted arylene, optionally substituted heteroarylene, or a combination thereof. In certain embodiments, L is a bond. In certain embodiments, L is optionally substituted alkylene. In certain embodiments, L is optionally substituted alkenylene. In certain embodiments, L is optionally substituted alkynylene. In certain embodiments, L is optionally substituted heteroalkylene. In certain embodiments, L is optionally substituted heteroalkenylene. In certain embodiments, L is optionally substituted heteroalkynylene. In certain embodiments, L is optionally substituted heterocyclylene. In certain embodiments, L is optionally substituted carbocyclylene. In certain embodiments, L is optionally substituted arylene. In certain embodiments, L is optionally substituted heteroarylene.

In certain embodiments, L is optionally substituted alkylene. In certain embodiments, L is optionally substituted C₁₋₁₂ alkylene. In certain embodiments, L is optionally substituted C₁₋₆ alkylene. In certain embodiments, L is unsubstituted C₁₋₆ alkylene. In certain embodiments, L is substituted C₁₋₆ alkylene. In certain embodiments, L is optionally substituted methylene, optionally substituted ethylene, optionally substituted n-propylene, optionally substituted isopropylene, optionally substituted n-butylene, optionally substituted tert-butylene, optionally substituted sec-butylene, optionally substituted isobutylene, optionally substituted n-pentylene, optionally substituted 3-pentanylene, optionally substituted amylene, optionally substituted neopentylene, optionally substituted 3-methylene-2-butanylene, optionally substituted tert-amylene, or optionally substituted n-hexylene. In certain embodiments, L is substituted methylene, substituted ethylene, substituted n-propylene, substituted isopropylene, substituted n-butylene, substituted tert-butylene, substituted sec-butylene, substituted isobutylene, substituted n-pentylene, substituted 3-pentanylene, substituted amylene, substituted neopentylene, substituted 3-methylene-2-butanylene, substituted tert-amylene, or substituted n-hexylene. In certain embodiments, L is unsubstituted methylene, unsubstituted ethylene, unsubstituted n-propylene, unsubstituted isopropylene, unsubstituted n-butylene, unsubstituted tert-butylene, unsubstituted sec-butylene, unsubstituted isobutylene, unsubstituted n-pentylene, unsubstituted 3-pentanylene, unsubstituted amylene, unsubstituted neopentylene, unsubstituted 3-methylene-2-butanylene, unsubstituted tert-amylene, or unsubstituted n-hexylene. In certain embodiments, L is optionally substituted methylene, optionally substituted ethylene, optionally substituted n-propylene, optionally substituted n-butylene, optionally substituted n-pentylene, or optionally substituted n-hexylene. In certain embodiments, L is substituted methylene, substituted ethylene, substituted n-propylene, substituted n-butylene, substituted n-pentylene, or substituted n-hexylene. In certain embodiments, L is unsubstituted methylene, unsubstituted ethylene, unsubstituted n-propylene, unsubstituted n-butylene, unsubstituted n-pentylene, or unsubstituted n-hexylene. In certain embodiments, L is optionally substituted n-propylene. In certain embodiments, L is substituted n-propylene. In certain embodiments, L is unsubstituted n-propylene.

In certain embodiments, L is

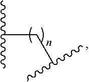

wherein n is 1, 2, 3, 4, or 5. In certain embodiments, L is

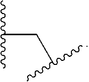 [SMILES: *C*]

In certain embodiments, L is

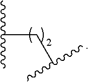

In certain embodiments, L is

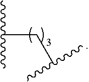

In certain embodiments, L is

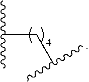

In certain embodiments, L is

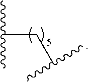

In certain embodiments, the compound of Formula (I) is of Formula (I-b-i):

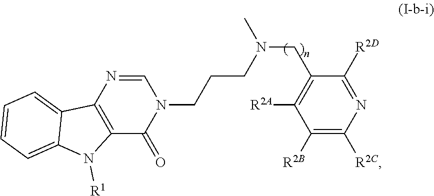

or a pharmaceutically acceptable salt, solvate, hydrate, polymorph, co-crystal, tautomer, stereoisomer, isotopically labeled compound, or prodrug thereof, wherein:
each of R^{2A}, R^{2B}, R^{2C}, and R^{2D} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom or atoms to form an optionally substituted heterocyclic ring or optionally substituted heteroaryl ring; and
n is 1, 2, 3, 4, or 5.

In certain embodiments, n is 1, 2, 3, 4, or 5. In certain embodiments, n is 1. In certain embodiments, n is 2. In certain embodiments, n is 3. In certain embodiments, n is 4. In certain embodiments, n is 5.

In certain embodiments, the compound of Formula (I-b-i) is of Formula (I-b-ii):

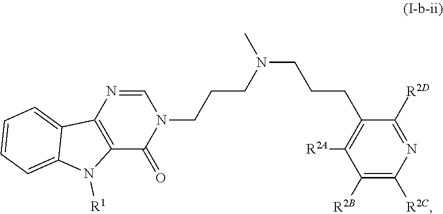

or a pharmaceutically acceptable salt, solvate, hydrate, polymorph, co-crystal, tautomer, stereoisomer, isotopically labeled compound, or prodrug thereof.

In certain embodiments, the compound of Formula (I-b-i) is of Formula (I-b-iii):

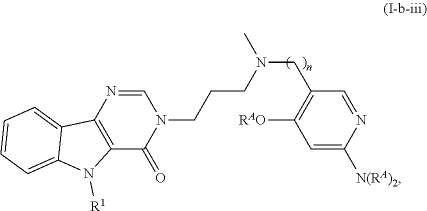

or a pharmaceutically acceptable salt, solvate, hydrate, polymorph, co-crystal, tautomer, stereoisomer, isotopically labeled compound, or prodrug thereof.

In certain embodiments, the compound of Formula (I-b-i) is of Formula (I-b-iv):

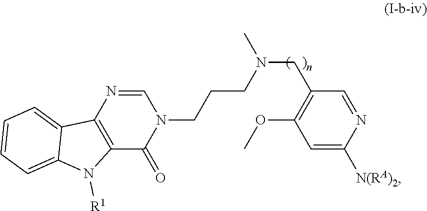

or a pharmaceutically acceptable salt, solvate, hydrate, polymorph, co-crystal, tautomer, stereoisomer, isotopically labeled compound, or prodrug thereof.

In certain embodiments, the compound of Formula (I-b-i) is of Formula (I-b-v):

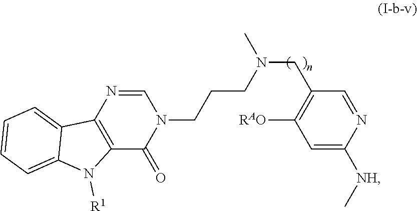

or a pharmaceutically acceptable salt, solvate, hydrate, polymorph, co-crystal, tautomer, stereoisomer, isotopically labeled compound, or prodrug thereof.

In certain embodiments, the compound of Formula (I-b-i) is of Formula (I-b-vi):

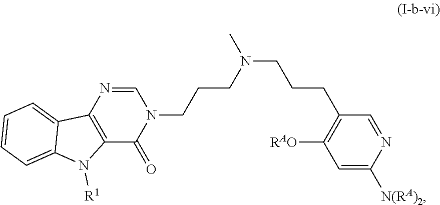

or a pharmaceutically acceptable salt, solvate, hydrate, polymorph, co-crystal, tautomer, stereoisomer, isotopically labeled compound, or prodrug thereof.

In certain embodiments, the compound of Formula (I-b-i) is of Formula (I-b-vii):

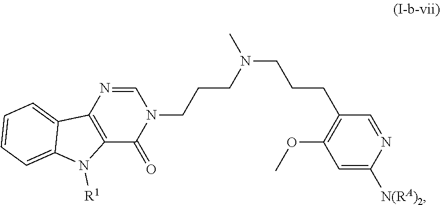

or a pharmaceutically acceptable salt, solvate, hydrate, polymorph, co-crystal, tautomer, stereoisomer, isotopically labeled compound, or prodrug thereof.

In certain embodiments, the compound of Formula (I-b-i) is of Formula (I-b-viii):

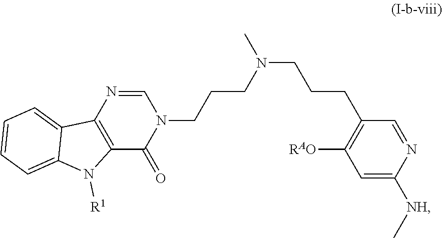

or a pharmaceutically acceptable salt, solvate, hydrate, polymorph, co-crystal, tautomer, stereoisomer, isotopically labeled compound, or prodrug thereof.

In certain embodiments, each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring. In certain embodiments, at least one occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring. In certain embodiments, at least one occurrence of R^{A} is hydrogen. In certain embodiments, at least one occurrence of R^{A} is optionally substituted acyl. In certain embodiments, at least one occurrence of R^{A} is optionally substituted C₁₋₁₂ alkyl. In certain embodiments, at least one occurrence of R^{A} is optionally substituted C₁₋₆ alkyl. In certain embodiments, at least one occurrence of R^{A} is unsubstituted C₁₋₆ alkyl. In certain embodiments, at least one occurrence of R^{A} is substituted C₁₋₆ alkyl. In certain embodiments, at least one occurrence of R^{A} is optionally substituted methyl, optionally substituted ethyl, optionally substituted n-propyl, optionally substituted isopropyl, optionally substituted n-butyl, optionally substituted tert-butyl, optionally substituted sec-butyl, optionally substituted isobutyl, optionally substituted n-pentyl, optionally substituted 3-pentanyl, optionally substituted amyl, optionally substituted neopentyl, optionally substituted 3-methyl-2-butanyl, optionally substituted tert-amyl, or optionally substituted n-hexyl. In certain embodiments, at least one occurrence of R^{A} is optionally substituted C₂₋₁₂ alkenyl. In certain embodiments, at least one occurrence of R^{A} is optionally substituted C₂₋₆ alkenyl. In certain embodiments, at least one occurrence of R^{A} is optionally substituted ethenyl, optionally substituted 1-propenyl, optionally substituted 2-propenyl, optionally substituted 1-butenyl, optionally substituted 2-butenyl, optionally substituted butadienyl, optionally substituted pentenyl, optionally substituted pentadienyl, or optionally substituted hexenyl. In certain embodiments, at least one occurrence of R^{A} is optionally substituted C₂₋₁₂ alkynyl. In certain embodiments, at least one occurrence of R^{A} is optionally substituted C₂₋₆ alkynyl. In certain embodiments, at least one occurrence of R^{A} is optionally substituted ethynyl, optionally substituted 1-propynyl, optionally substituted 2-propynyl, optionally substituted 1-butynyl, optionally substituted 2-butynyl, optionally substituted pentynyl, or optionally substituted hexynyl. In certain embodiments, at least one occurrence of R^{A} is optionally substituted heteroC₁₋₁₂ alkyl. In certain embodiments, at least one occurrence of R^{A} is optionally substituted heteroC₁₋₆ alkyl. In certain embodiments, at least one occurrence of R^{A} is optionally substituted heteroC₁₋₁₂ alkenyl. In certain embodiments, at least one occurrence of R^{A} is optionally substituted heteroC₁₋₆ alkenyl. In certain embodiments, at least one occurrence of R^{A} is optionally substituted heteroC₁₋₁₂ alkynyl. In certain embodiments, at least one occurrence of R^{A} is optionally substituted heteroC₁₋₆ alkynyl. In certain embodiments, at least one occurrence of R^{A} is optionally substituted C₃ 14 cycloalkyl. In certain embodiments, at least one occurrence of R^{A} is optionally substituted 5-10 membered heterocyclyl. In certain embodiments, at least one occurrence of R^{A} is optionally substituted 6-14 membered aryl. In certain embodiments, at least one occurrence of R^{A} is optionally substituted 5-14 membered heteroaryl. In certain embodiments, at least one occurrence of R^{A} is a nitrogen protecting group when attached to a nitrogen atom. In certain embodiments, at least one occurrence of R^{A} is an oxygen protecting group when attached to an oxygen atom. In certain embodiments, at least one occurrence of R^{A} is a sulfur protecting group when attached to a sulfur atom. In certain embodiments, at least two occurrences of R^{A} are joined together with their intervening atom to form an optionally substituted 5-10 membered heterocyclic ring. In certain embodiments, at least two occurrences of R^{A} are joined together with their intervening atom to form an optionally substituted 5-14 membered heteroaryl ring.

In certain embodiments, the compound is of formula:

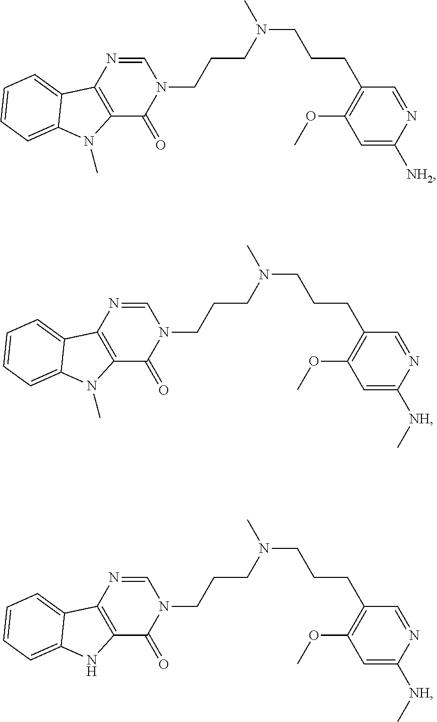 [SMILES: COc1cc(N)ncc1CCCN(C)CCCn1cnc2c3ccccc3n(C)c2c1=O ; CNc1cc(OC)c(CCCN(C)CCCn2cnc3c4ccccc4n(C)c3c2=O)cn1 ; CNc1cc(OC)c(CCCN(C)CCCn2cnc3c([nH]c4ccccc43)c2=O)cn1]

or a pharmaceutically acceptable salt, solvate, hydrate, polymorph, co-crystal, tautomer, stereoisomer, isotopically labeled compound, or prodrug thereof.

In another aspect, the present disclosure provides compounds of formula:

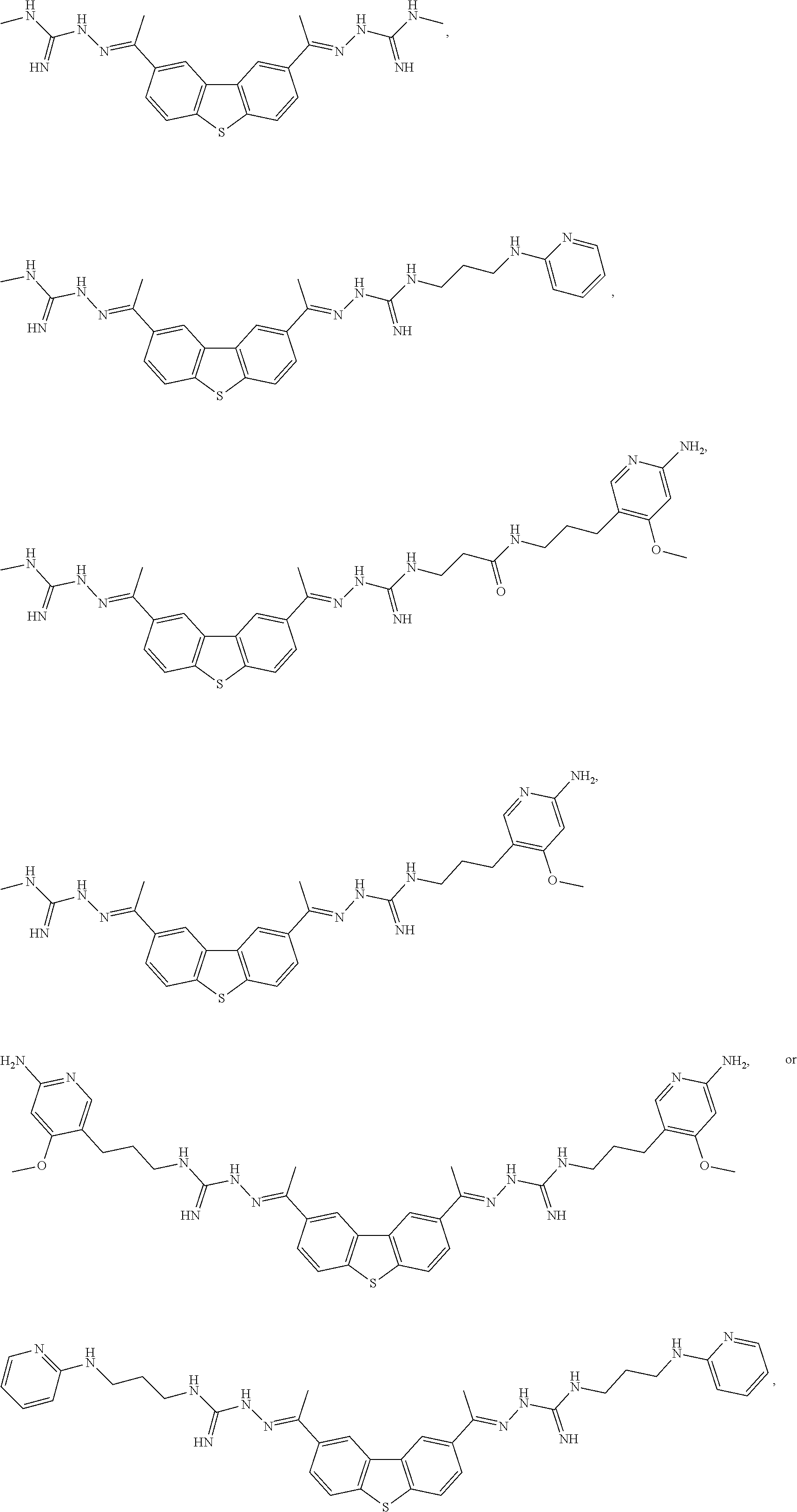

or a pharmaceutically acceptable salt, solvate, hydrate, polymorph, co-crystal, tautomer, stereoisomer, isotopically labeled compound, or prodrug thereof.

In another aspect, the present disclosure provides a compound of formula:

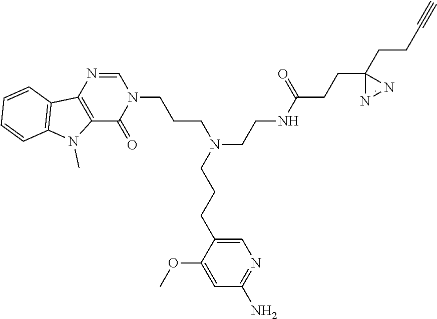 [SMILES: C#CCCC1(CCC(=O)NCCN(CCCc2cnc(N)cc2OC)CCCn2cnc3c4ccccc4n(C)c3c2=O)N=N1]

or a pharmaceutically acceptable salt, solvate, hydrate, polymorph, co-crystal, tautomer, stereoisomer, isotopically labeled compound, or prodrug thereof.

In certain embodiments, a provided compound (a compound described herein, a compound of the present disclosure) is a compound of any of the formulae herein (e.g., Formula (I)) or pharmaceutically acceptable salt, solvate, hydrate, polymorph, co-crystal, tautomer, stereoisomer, isotopically labeled compound, or prodrug thereof. In certain embodiments, a provided compound is a compound of any of the formulae herein (e.g., Formula (I)), or a pharmaceutically acceptable salt or tautomer thereof. In certain embodiments, a provided compound is a compound of any of the formulae herein (e.g., Formula (I)), or a pharmaceutically acceptable salt thereof. In certain embodiments, a provided compound is a compound of any of the formulae herein (e.g., Formula (I)), or a salt thereof.

### Pharmaceutical Compositions and Kits

In one aspect, the present disclosure provides pharmaceutical compositions comprising a provided compound. In some embodiments, the pharmaceutical composition comprises one or more excipients. In certain embodiments, the pharmaceutical compositions described herein comprise a provided compound and an excipient.

In certain embodiments, the pharmaceutical composition comprises an effective amount of the provided compound. In certain embodiments, the effective amount is a therapeutically effective amount. In certain embodiments, the effective amount is a prophylactically effective amount.

In certain embodiments, the effective amount is an amount effective for stabilizing an RNA target in a subject in need thereof or in a cell, tissue, or biological sample. In certain embodiments, the RNA target is microtubule-associated protein Tau (MAPT) pre-mRNA. In certain embodiments, the effective amount is an amount effective for decreasing a ratio of an amount of a first mRNA isoform to an amount of a second mRNA isoform in a subject in need thereof or in a cell, tissue, or biological sample. In certain embodiments, the effective amount is an amount effective for treating a disease or disorder associated with microtubule-associated protein Tau (MAPT) pre-mRNA (e.g., a neurodegenerative disease (e.g., frontotemporal dementia and parkinsonism linked to chromosome 17 (FTDP-17), Alzheimer's disease)) in a subject in need thereof. In certain embodiments, the effective amount is an amount effective for preventing a disease or disorder associated with MAPT pre-mRNA (e.g., a neurodegenerative disease (e.g., frontotemporal dementia and parkinsonism linked to chromosome 17 (FTDP-17), Alzheimer's disease)) in a subject in need thereof. In certain embodiments, the effective amount is an amount effective for reducing the risk of developing a disease or disorder associated with MAPT pre-mRNA (e.g., a neurodegenerative disease (e.g., frontotemporal dementia and parkinsonism linked to chromosome 17 (FTDP-17), Alzheimer's disease)) in a subject in need thereof.

In certain embodiments, the subject is an animal. In certain embodiments, the subject is a human. In certain embodiments, the subject is a human aged 18 years or older. In certain embodiments, the subject is a human aged 12-18 years, exclusive. In certain embodiments, the subject is a human aged 2-12 years, inclusive. In certain embodiments, the subject is a human younger than 2 years. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for decreasing an amount of microtubule-associated protein Tau (MAPT) pre-mRNA (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the effective amount is an amount effective for decreasing an amount of MAPT pre-mRNA by a range between a percentage described in this paragraph and another percentage described in this paragraph, inclusive.

In certain embodiments, the pharmaceutical composition is for use in treating a disease or disorder associated with microtubule-associated protein Tau (MAPT) pre-mRNA (e.g., a neurodegenerative disease (e.g., frontotemporal dementia and parkinsonism linked to chromosome 17 (FTDP-17), Alzheimer's disease)) in a subject in need thereof. In certain embodiments, the pharmaceutical composition is for use in preventing a disease or disorder associated with MAPT pre-mRNA (e.g., a neurodegenerative disease (e.g., frontotemporal dementia and parkinsonism linked to chromosome 17 (FTDP-17), Alzheimer's disease)) in a subject in need thereof. In certain embodiments, the pharmaceutical composition is for use in stabilizing an RNA target in a subject in need thereof or in a cell, tissue, or biological sample. In certain embodiments, the pharmaceutical composition is for use in decreasing a ratio of an amount of a first mRNA isoform to an amount of a second mRNA isoform in a subject in need thereof or in a cell, tissue, or biological sample.

A provided compound or pharmaceutical composition, as described herein, can be administered in combination with one or more additional pharmaceutical agents (e.g., therapeutically and/or prophylactically active agents). The provided compounds or pharmaceutical compositions can be administered in combination with additional pharmaceutical agents that improve their activity (e.g., activity (e.g., potency and/or efficacy) in treating a disease or disorder associated with microtubule-associated protein Tau (MAPT) pre-mRNA (e.g., a neurodegenerative disease (e.g., frontotemporal dementia and parkinsonism linked to chromosome 17 (FTDP-17), Alzheimer's disease)) in a subject in need thereof, in preventing a disease or disorder associated with MAPT pre-mRNA (e.g., a neurodegenerative disease (e.g., frontotemporal dementia and parkinsonism linked to chromosome 17 (FTDP-17), Alzheimer's disease)) in a subject in need thereof, and/or in reducing the risk of developing a disease or disorder associated with MAPT pre-mRNA (e.g., a neurodegenerative disease (e.g., frontotemporal dementia and parkinsonism linked to chromosome 17 (FTDP-17), Alzheimer's disease)) in a subject in need thereof, improve bioavailability, improve safety, reduce drug resistance, reduce and/or modify metabolism, inhibit excretion, and/or modify distribution in a subject or cell. It will also be appreciated that the additional pharmaceutical agents employed may achieve a desired effect for the same disorder, and/or it may achieve different effects. In certain embodiments, a pharmaceutical composition described herein including a provided compound described herein and an additional pharmaceutical agent exhibit a synergistic effect that is absent in a pharmaceutical composition including one of the provided compounds and the additional pharmaceutical agent, but not both. In some embodiments, the additional pharmaceutical agent achieves a desired effect for the same disorder. In some embodiments, the additional pharmaceutical agent achieves different effects.

The provided compound or pharmaceutical composition can be administered concurrently with, prior to, or subsequent to one or more additional pharmaceutical agents, which are different from the compound or pharmaceutical composition and may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides, synthetic proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease or disorder associated with microtubule-associated protein Tau (MAPT) pre-mRNA (e.g., a neurodegenerative disease (e.g., frontotemporal dementia and parkinsonism linked to chromosome 17 (FTDP-17), Alzheimer's disease)).

Each additional pharmaceutical agent may be administered at a dose and/or on a time schedule determined for that pharmaceutical agent. The additional pharmaceutical agents may also be administered together with each other and/or with the compound or pharmaceutical composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins.

In certain embodiments, the provided compound or pharmaceutical composition is a solid. In certain embodiments, the provided compound or pharmaceutical composition is a powder. In certain embodiments, the provided compound or pharmaceutical composition can be dissolved in a liquid to make a solution. In certain embodiments, the provided compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

Pharmaceutical compositions described herein can be prepared by any method known in the art of pharmacology. In general, such preparatory methods include the steps of bringing the composition comprising a provided compound (i.e., the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the provided compound, pharmaceutically acceptable excipient, agent, and/or any additional ingredients in a pharmaceutical composition described herein will vary, depending upon the identity, size, and/or condition of the subject treated and further depending upon the route by which the pharmaceutical composition is to be administered. The pharmaceutical composition may comprise between 0.1% and 100% (w/w) agent, inclusive.

Pharmaceutically acceptable excipients used in manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients and accessory ingredients, such as cocoa butter and suppository waxes, coloring agents, coating agents, sweetening, flavoring, and perfuming agents, may also be present in the pharmaceutical composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan monostearate (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral pharmaceutical compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid pharmaceutical compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Pharmaceutical compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

Suitable devices for use in delivering injectable pharmaceutical compositions described herein include short needle devices. Injectable pharmaceutical compositions can be administered by devices which limit the effective penetration length of a needle into the skin. Alternatively or additionally, conventional syringes can be used in the classical mantoux method of administration. Jet injection devices which deliver liquid formulations via a liquid jet injector and/or via a needle. Ballistic powder/particle delivery devices which use compressed gas to accelerate the compound in powder form are suitable.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such pharmaceutical compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder pharmaceutical compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the pharmaceutical composition, and the active ingredient may constitute 0.1 to 20% (w/w) of the pharmaceutical composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such pharmaceutical compositions are generally suitable for administration to animals of all sorts. Modification of pharmaceutical compositions suitable for administration to humans in order to render the pharmaceutical compositions suitable for administration to various animals is well understood, and the ordinarily skilled veterinary pharmacologist can design and/or perform such modification with ordinary experimentation.

Provided compounds are typically formulated in dosage unit form for ease of administration and uniformity of dosage. It will be understood, however, that the total daily usage of the pharmaceutical compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The provided compounds and pharmaceutical compositions provided herein can be administered by any route, including enteral (e.g., oral), parenteral, intravenous, intramuscular, intraarticular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically, contemplated routes are intraarticular administration, oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a provided compound required to achieve an effective amount will vary from subject to subject, depending, for example, on species, age, and general condition of a subject, severity of the side effects or disorder, identity of the particular compound of the disclosure, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of an agent described herein.

In certain embodiments, a pharmaceutical composition comprising a provided compound is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the pharmaceutical composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Dose ranges as described herein provide guidance for the administration of provided compounds or pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

In certain embodiments, when multiple doses are administered to a subject or applied to a tissue or cell, the frequency of administering the multiple doses to the subject or applying the multiple doses to the tissue or cell may be, in non-limiting examples, three doses a day, two doses a day, one dose a day, one dose every other day, one dose every third day, one dose every week, one dose every two weeks, one dose every three weeks, or one dose every four weeks, or even slow dose controlled delivery over a selected period of time using a drug delivery device. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

Also encompassed by the present disclosure are kits (e.g., pharmaceutical packs). In certain embodiments, the kit comprises a provided compound or pharmaceutical composition described herein, and instructions for using the compound or pharmaceutical composition. In certain embodiments, the kit comprises a first container, wherein the first container includes the compound or pharmaceutical composition. In some embodiments, the kit further comprises a second container. In certain embodiments, the second container includes an excipient (e.g., an excipient for dilution or suspension of the compound or pharmaceutical composition). In certain embodiments, the second container includes an additional pharmaceutical agent. In some embodiments, the kit further comprises a third container. In certain embodiments, the third container includes an additional pharmaceutical agent. In some embodiments, the provided compound or pharmaceutical composition included in the first container and the excipient or additional pharmaceutical agent included in the second container are combined to form one unit dosage form. In some embodiments, the provided compound or pharmaceutical composition included in the first container, the excipient included in the second container, and the additional pharmaceutical agent included in the third container are combined to form one unit dosage form. In certain embodiments, each of the first, second, and third containers is independently a vial, ampule, bottle, syringe, dispenser package, tube, or inhaler.

In certain embodiments, the instructions are for administering the provided compound or pharmaceutical composition to a subject (e.g., a subject in need of treatment or prevention of a disease described herein). In certain embodiments, the instructions are for contacting a biological sample or cell with the provided compound or pharmaceutical composition. In certain embodiments, the instructions comprise information required by a regulatory agency, such as the U.S. Food and Drug Administration (FDA) or the European Agency for the Evaluation of Medicinal Products (EMA). In certain embodiments, the instructions comprise prescribing information.

In certain embodiments, the kits and instructions provide for treating a disease or disorder associated with microtubule-associated protein Tau (MAPT) pre-mRNA (e.g., a neurodegenerative disease (e.g., frontotemporal dementia and parkinsonism linked to chromosome 17 (FTDP-17), Alzheimer's disease)) in a subject in need thereof. In certain embodiments, the kits and instructions provide for preventing a disease or disorder associated with MAPT pre-mRNA (e.g., a neurodegenerative disease (e.g., frontotemporal dementia and parkinsonism linked to chromosome 17 (FTDP-17), Alzheimer's disease)) in a subject in need thereof. In certain embodiments, the kits and instructions provide for reducing the risk of developing a disease or disorder associated with MAPT pre-mRNA (e.g., a neurodegenerative disease (e.g., frontotemporal dementia and parkinsonism linked to chromosome 17 (FTDP-17), Alzheimer's disease)) in a subject in need thereof. In certain embodiments, the kits and instructions provide for stabilizing an RNA target in a subject in need thereof or in a cell, tissue, or biological sample. In certain embodiments, the kits and instructions provide for decreasing a ratio of an amount of a first mRNA isoform to an amount of a second mRNA isoform in a subject in need thereof or in a cell, tissue, or biological sample.

A kit described herein may include one or more additional pharmaceutical agents described herein as a separate pharmaceutical composition.

Another object of the present disclosure is the use of a compound as described herein in the manufacture of a medicament for use in the treatment of a disorder or disease described herein. Another object of the present disclosure is the use of a compound as described herein for use in the treatment of a disorder or disease described herein.

### Methods of Treatment and Prevention

In another aspect, the present disclosure provides methods of treating or preventing a disease in a subject in need thereof, comprising administering to the subject in need thereof a provided compound or pharmaceutical composition. In certain embodiments, the present disclosure provides methods of treating a disease in a subject in need thereof, comprising administering to the subject in need thereof a provided compound or pharmaceutical composition. In certain embodiments, the present disclosure provides methods of preventing a disease in a subject in need thereof, comprising administering to the subject in need thereof a provided compound or pharmaceutical composition. In certain embodiments, the disease is associated with microtubule-associated protein Tau (MAPT) pre-mRNA (e.g., a neurodegenerative disease (e.g., frontotemporal dementia and parkinsonism linked to chromosome 17 (FTDP-17), Alzheimer's disease)).

In another aspect, the present disclosure provides a provided compound or pharmaceutical composition for use in treating or preventing a disease in a subject in need thereof. In another aspect, the present disclosure provides a provided compound or pharmaceutical composition for use in treating a disease in a subject in need thereof. In another aspect, the present disclosure provides a provided compound or pharmaceutical composition for use in preventing a disease in a subject in need thereof. In certain embodiments, the disease is associated with microtubule-associated protein Tau (MAPT) pre-mRNA (e.g., a neurodegenerative disease (e.g., frontotemporal dementia and parkinsonism linked to chromosome 17 (FTDP-17), Alzheimer's disease)).

In another aspect, the present disclosure provides a provided compound or pharmaceutical composition for use in the manufacture of a medicament for treatment or prevention of a disease in a subject in need thereof. In another aspect, the present disclosure provides a provided compound or pharmaceutical composition for use in the manufacture of a medicament for treatment of a disease in a subject in need thereof. In another aspect, the present disclosure provides a provided compound or pharmaceutical composition for use in the manufacture of a medicament for prevention of a disease in a subject in need thereof. In certain embodiments, the disease is associated with microtubule-associated protein Tau (MAPT) pre-mRNA (e.g., a neurodegenerative disease (e.g., frontotemporal dementia and parkinsonism linked to chromosome 17 (FTDP-17), Alzheimer's disease)).

In certain embodiments, the disease is associated with microtubule-associated protein Tau (MAPT) pre-mRNA. In certain embodiments, the disease is a neurodegenerative disease (e.g., frontotemporal dementia and parkinsonism linked to chromosome 17 (FTDP-17), Alzheimer's disease). In certain embodiments, the neurodegenerative disease is frontotemporal dementia and parkinsonism linked to chromosome 17 (FTDP-17). In certain embodiments, the neurodegenerative disease is Alzheimer's disease. In certain embodiments, the method comprises mitigating pathologies relative to Tauopathy. In certain embodiments, the method comprises correcting aberrant behavior phenotypes associated with the disease.

Methods of Stabilizing an RNA Target and Decreasing a Ratio of an Amount of a First mRNA Isoform to an Amount of a Second mRNA Isoform

In another aspect, the present disclosure provides methods of stabilizing an RNA target in a subject in need thereof or in a cell, tissue, or biological sample, comprising administering to the subject in need thereof or contacting the cell, tissue, or biological sample with an effective amount of a provided compound or composition. In certain embodiments, the present disclosure provides methods of stabilizing an RNA target in a subject in need thereof, comprising administering to the subject in need thereof an effective amount of a provided compound or composition. In certain embodiments, the present disclosure provides methods of stabilizing an RNA target in a cell, tissue, or biological sample, comprising contacting the cell, tissue, or biological sample with an effective amount of a provided compound or composition. In certain embodiments, the present disclosure provides a provided compound or composition for use in stabilizing an RNA target in a subject in need thereof or in a cell, tissue, or biological sample. In certain embodiments, the present disclosure provides a provided compound or composition for use in the manufacture of a medicament for stabilizing an RNA target in a subject in need thereof or in a cell, tissue, or biological sample.

In certain embodiments, the RNA target is microtubule-associated protein Tau (MAPT) pre-mRNA. In certain embodiments, stabilizing the RNA target comprises intercalating to an A bulge site of the MAPT pre-MRNA. In certain embodiments, stabilizing the RNA target comprises forming a base triple with a GC pair of the MAPT pre-MRNA. In certain embodiments, stabilizing the RNA target comprises inducing exon 10 skipping.

In another aspect, the present disclosure provides methods of decreasing a ratio of an amount of a first mRNA isoform to an amount of a second mRNA isoform in a subject in need thereof or in a cell, tissue, or biological sample, comprising administering to the subject in need thereof or contacting the cell, tissue, or biological sample with an effective amount of a provided compound or composition. In certain embodiments, the present disclosure provides methods of decreasing a ratio of an amount of a first mRNA isoform to an amount of a second mRNA isoform in a subject in need thereof, comprising administering to the subject in need thereof an effective amount of a provided compound or composition. In certain embodiments, the present disclosure provides methods of decreasing a ratio of an amount of a first mRNA isoform to an amount of a second mRNA isoform in a cell, tissue, or biological sample, comprising contacting the cell, tissue, or biological sample with an effective amount of a provided compound or composition. In certain embodiments, the present disclosure provides a provided compound or composition for use in decreasing a ratio of an amount of a first mRNA isoform to an amount of a second mRNA isoform in a subject in need thereof or in a cell, tissue, or biological sample. In certain embodiments, the present disclosure provides a provided compound or composition for use in the manufacture of a medicament for decreasing a ratio of an amount of a first mRNA isoform to an amount of a second mRNA isoform in a subject in need thereof or in a cell, tissue, or biological sample.

In certain embodiments, the method comprises decreasing the amount of the first mRNA isoform (e.g., by at least about 10%, at least about 20%, at least about 30%, at least about 40%, at least about 50%, at least about 60%, at least about 70%, at least about 80%, at least about 90%, at least about 95%, at least about 98%, at least about 99%, or at least about 100%). In certain embodiments, the first mRNA isoform is four-repeat Tau mRNA (4R-Tau mRNA). In certain embodiments, the second mRNA isoform is three-repeat Tau mRNA (3R-Tau mRNA).

In certain embodiments, the method comprises decreasing a ratio of an amount of a first protein isoform to an amount of a second protein isoform. In certain embodiments, the method comprises decreasing the amount of the first protein isoform (e.g., by at least about 10%, at least about 20%, at least about 30%, at least about 40%, at least about 50%, at least about 60%, at least about 70%, at least about 80%, at least about 90%, at least about 95%, at least about 98%, at least about 99%, or at least about 100%). In certain embodiments, the first protein isoform is four-repeat Tau (4R-Tau). In certain embodiments, the second protein isoform is three-repeat Tau (3R-Tau).

In certain embodiments, the cell, tissue, or biological sample is in vivo. In certain embodiments, the cell, tissue, or biological sample is in vitro.

### Methods of Preparation

In another aspect, the present disclosure provides methods of preparing a compound of Formula (I-a-v):

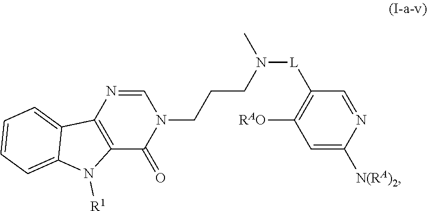

or a pharmaceutically acceptable salt, solvate, hydrate, polymorph, co-crystal, tautomer, stereoisomer, isotopically labeled compound, or prodrug thereof, comprising reacting a compound of Formula (II):

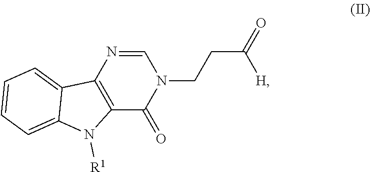

or a salt thereof, with a compound of Formula (III):

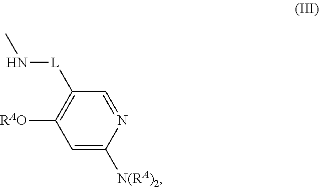

or a salt thereof, wherein:
R¹ is hydrogen or optionally substituted alkyl;
L is linker; and
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom, or two occurrences of R^{A} are joined together with their intervening atom to form an optionally substituted heterocyclic ring or optionally substituted heteroaryl ring.

In certain embodiments, the compound of Formula (II) is of Formula (II-a):

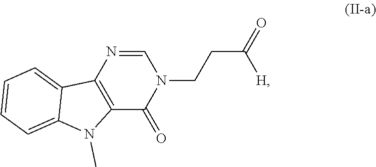

or salt thereof.

In certain embodiments, the compound of Formula (II) is of Formula (II-b):

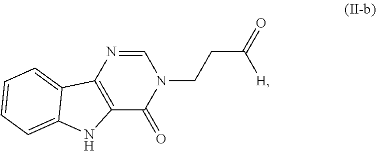

or salt thereof.

In certain embodiments, the compound of Formula (III) is of Formula (III-a):

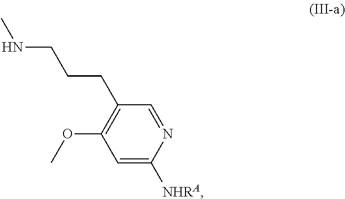

or a salt thereof, wherein R^{A} is a nitrogen protecting group. In certain embodiments, the method further comprises deprotecting the nitrogen protecting group. In certain embodiments, the nitrogen protecting group is Boc (tert-butyloxycarbonyl).

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1: Design of an Orally Bioavailable Small Molecule that Modulates Tau Pre-mRNA Splicing

Design and synthesis of ligands with high affinity for the tau exon 10-intron junction hairpin structure. A framework was previously reported to design and optimize drug-like small molecules that bind tau pre-mRNA and modulate the alternative splicing of exon 10,15 which encodes a microtubule binding domain. Initial molecules were designed from sequence to bind the A-bulge and its closing pairs present in the MAPT SRE12 using the lead identification strategy, Inforna.¹⁵ These molecules, with demonstrated binding to the target and cellular activity, informed a pharmacophore model that was used in virtual screen to generate a small molecule library with potential to modulate exon 10 splicing. Iterative biophysical, biochemical, and cellular studies afforded drug-like compounds that bound to tau pre-mRNA, and the most potent compound from these studies modulated splicing in primary mouse neurons harvested from tau mice.¹⁵

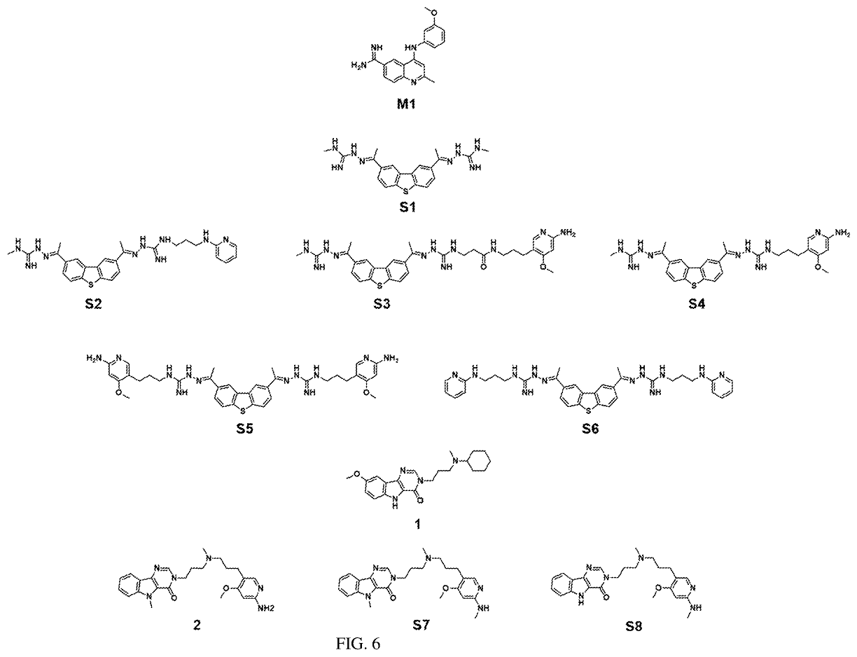

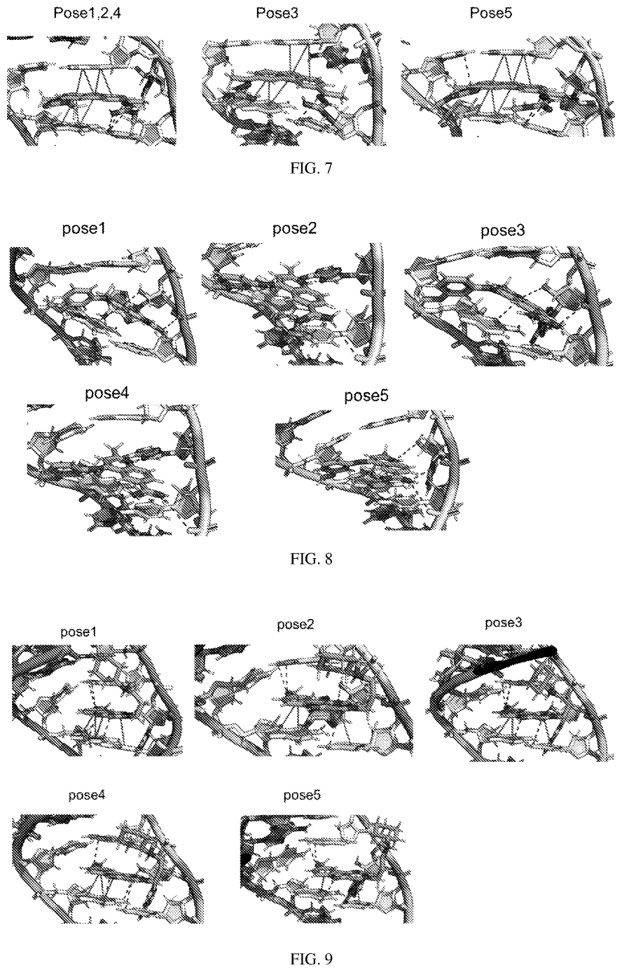

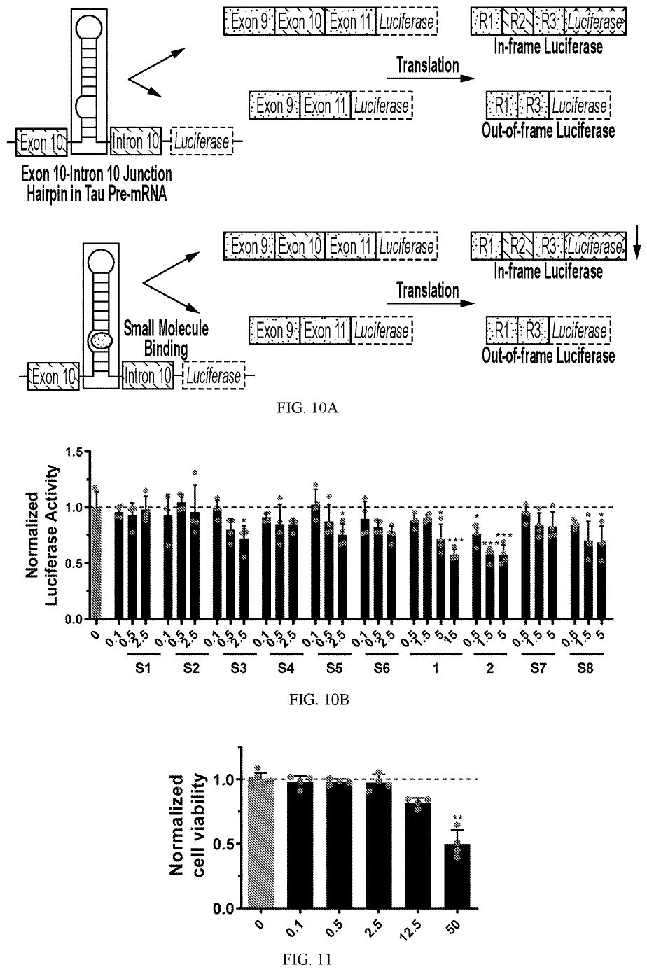

Three scaffold-diverse compounds M1, S1, and 1 (FIG. 6), emerged from these studies and their structures in complex with the tau pre-mRNA target were elucidated by nuclear magnetic resonance (NMR)-restrained molecular dynamics (PDB25 IDs: 6VA3 for M1, 6VA2 for S1 and 6VA4 for 1).¹⁵ Each small molecule has a central core that forms a network of interactions to the tau pre-mRNA SRE hairpin structure (FIGS. 7-9). Most of the interactions are hydrogen bonds to the closing GC base pairs of the A-bulge and stacking interactions with either one or both closing GC base pairs.

Further investigation of interaction patterns of the small molecules of the poses that comprise the NMR-restrained MD structures (20 structures for each small molecule) showed that M1 is stabilized solely by hydrogen bonds and S1 is stabilized by stacking interactions and hydrogen bonds. A web of interactions stabilizes the complex formed between 1 and the RNA, a combination of hydrogen bonds (backbone and bases) and stacking interactions with a calculated binding energy of −8.91 kcal/mol (Table 1). Further, central nervous system multiparameter optimization (CNS MPO) scores, which estimate blood-brain barrier (BBB) penetrance,²⁶ showed that 1 and M1 are likely to be BBB penetrant (CNS MPO=5.43 and 5.0, respectively) while S1 is likely not (CNS MPO=3.64). Although M1 shows a high CNS MPO score, it has disadvantages compared to 1 and S1 including a low calculated binding energy (−6.39 kcal/mol, Table 1) due to its weak interactions (solely hydrogen bonds) with the A-bulge binding pocket.¹⁵ In contrast, S1 has a low calculated binding energy (−11.42 kcal/mol; Table 1), despite its low CNS MPO score. As S1 and 1 are different chemical scaffolds with favorable binding energies, they were advanced for further optimization.

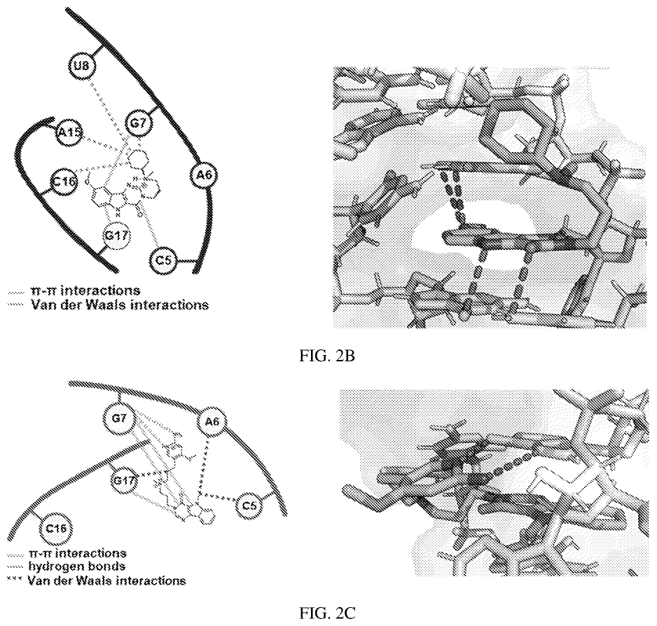

Careful inspection of the occupied area by S1 and 1 showed any growth along either the X⁻ or Y-axis would cause steric clashes with the surrounding atoms with no benefits in providing additional interactions (FIG. 2A; right). Therefore, molecular weight and functionalities were added to form additional stabilizing interactions with the RNA target along in the Z-axis, where the structures of the compounds bound to the MAPT SRE suggested an opportunity to form additional stabilizing interactions to the Hoogsteen face of the guanosine residue in the A-bulge's closing GC base pair.

RNA tertiary structure is the three-dimensional arrangement of RNA building blocks. Base triple is one type of RNA tertiary structure, which occurs when three nucleobases interacting edge-to-edge by hydrogen bonding.²⁷ To design a module capable of binding to the A-bulge's closing GC base pair, how nature evolves molecular recognition of RNA's major and minor grooves and triplex forming oligonucleotides was examined.²⁸⁻²⁹ For example, 2-aminopyridines form base triples with GC pairs,²¹ and its protonated state under physiological conditions increases its affinity for the negatively charged RNA. Therefore, 2-aminopyridine modules were appended onto the central cores of S1 and 1 to investigate if precise positioning of base triple could improve molecular recognition.

As a first step in the optimization process, eight analogs were designed, (FIG. 6, five based on S1 and three based on 1) guided by the previously elucidated NMR solution structures of the parent compounds in complex with the RNA. Notably, structures of the 1-RNA complex revealed that the cyclohexane moiety of 1 does not contribute to stacking interactions (FIG. 2B). Therefore, it provides a site that can be modified with a 2-aminopyridine module. Altogether, the 2-aminopyridine was attached to the both parent compounds with a flexible linker such that the linker to the RNA-binding molecule lies within the major groove and the 2-aminopyridine is positioned so that it can potentially form a base triple with the A-bulge's closing GC pair (FIGS. 2, 6). Three out of five analogs of S1 have a 2-aminopyridine module on one side (S2, S3, and S4) while the other two have a 2-aminopyridine module on both sides of S1 (S5 and S6). Analogs S2 and S6 have core binders linked to amino groups of 2-aminopyridine while the other six analogs have core binders linked to aromatic ring carbon atom of 2-aminopyridine. All three analogs of 1 have cyclohexane moiety replaced by a 2-aminopyridine module, which differs by a methyl group on the core binder or 2-aminopyridine.

Small molecule docking was next used to investigate the interactions formed between the MAPT SRE and the eight 2-aminopyridine conjugates. The RNA target was prepared by removing 1 from the RNA-ligand complex (6VA4) due to high similarity to S1 (6VA2) binding pocket (an RMSD value less than 2 Å). Derivatives around the S1 had the highest binding energies (Table 1), as compared to the derivatives of 1, the same pattern was observed with the core binders. Inspection of the lowest energy docked poses of 1 showed the appropriateness of the linker length placing the 2-aminopyridine ring in the vicinity of GC base pairs. In contrast, small molecule docking indicated that the S1 derivatives were unlikely to form base triple interactions between the 2-aminpyridine and the GC base pairs mainly due to the longer linker length and thus more rotational degrees of freedom. It was also observed that the longer linker length in S3 and S6 cause non-specific interactions of the linker with RNA backbone and pushing the core binder out of the binding pocket, further implicating linker length in base triple formation.

As a final step in the design process, CNS MPO scores were calculated for each analog, where addition of 2-aminopyridine to either 1 or S1 did not significantly reduce CNS MPO scores (Table 1). While the analogs of 1 gave CNS MPO scores over 4.0 (likely to be BBB penetrant), the analogs of S1 gave CNS MPO scores below 3.0 (the CNS MPO for S1=3.64), indicating lower likelihood of BBB penetrance (Table 1). Despite the low CNS MPO scores and results for docking studies, the S1 analogs were subjected to downstream studies due to the strong interaction of S1 with the SRE RNA.

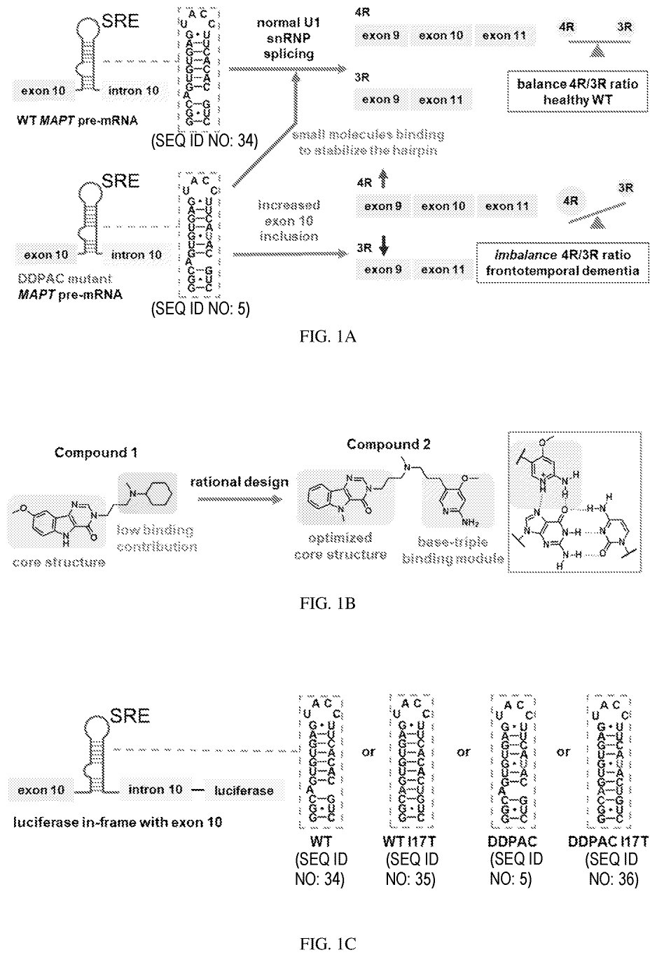

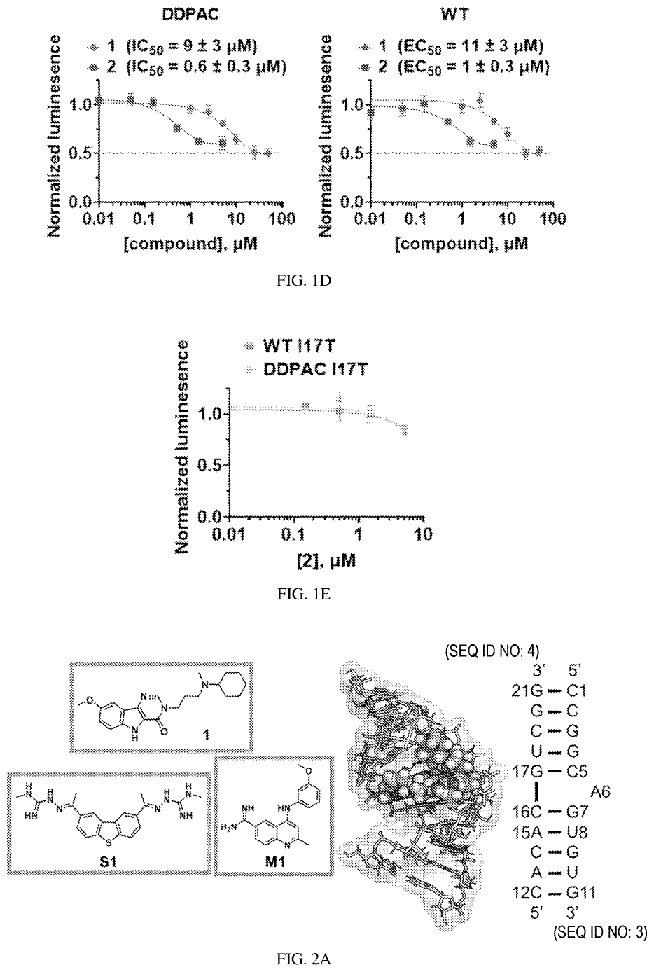

Bioactivity of 2-aminopyridine conjugates. The eight conjugates (five for S1 and three for 1) were screened in a cellular MAPT exon 10 splicing mini-gene reporter assay. The mini-gene comprises MAPT exons 9-11 with truncated introns and expresses firefly luciferase in-frame with exon 10, that is when exon 10 is included producing 4R tau (luciferase by proxy; FIG. 1C).¹⁰ Four reporters were used in these studies: (i) wild type (WT) MAPT; (ii) the DDPAC mutation; (iii) the single mutant, known as WT I17T, containing a U insertion opposite to the A-bulge (lacks the small molecule binding site); and (iv) the double mutant, known as DDPAC I17T (also lacks the small molecule binding site) (FIG. 1C).

Among eight conjugates using the DDPAC mini-gene, 2 excluded exon 10 to the greatest extent, as measured by the reduction of luciferase activity (FIG. 10B). An extended dose response afforded IC₅₀s of 9±3 μM and 0.6±0.3 μM for 1 and 2, respectively (FIG. 1D). A similar dose-dependent reduction in luciferase activity by 2 was also observed with the WT reporter (FIG. 1D). An attenuated response was observed for 2 for directing splicing of the mutant mini-genes, both WT I17T and DDPAC I17T which lack the A-bulge binding site, where activity was only observed at the highest dose (5 μM) and the observed reduction was around 15% (FIG. 1E). No effect on the viability of HeLa cells upon treatment with 2 was observed up to 12.5 μM, 2.5 times higher than its highest dose of 5 μM used in the luciferase assay, with a reduction of 17±3% (FIG. 11).

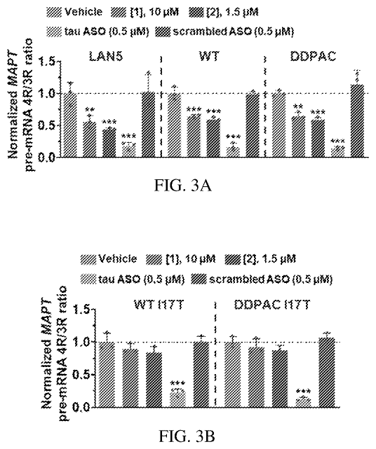

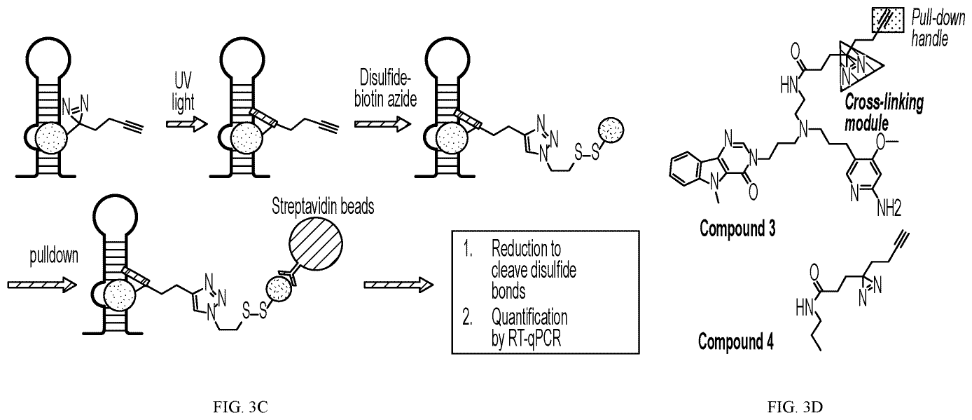

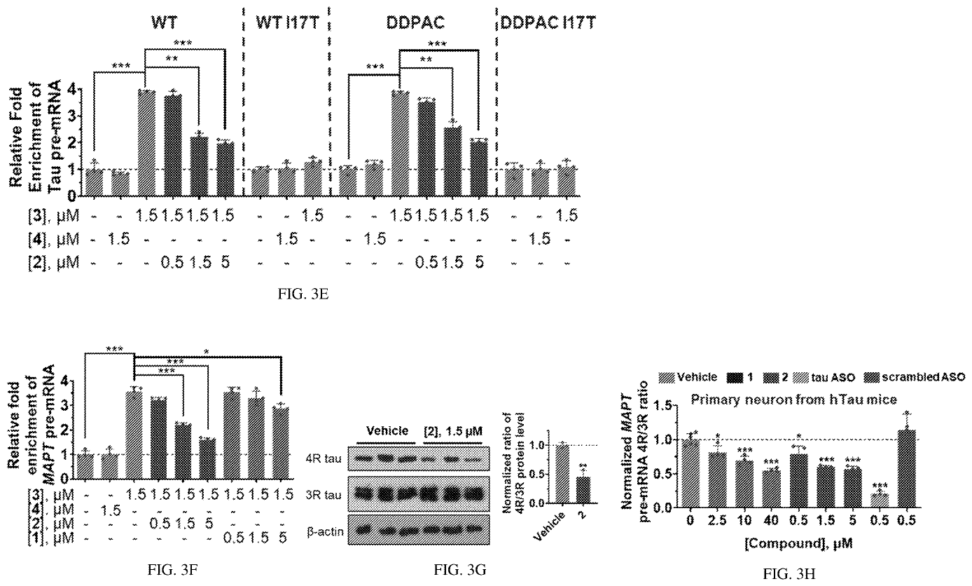

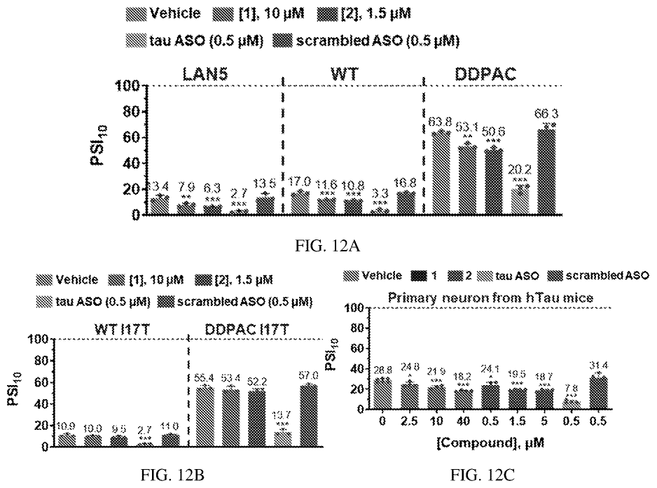

To demonstrate that the observed reduction of firefly luciferase activity was due to inhibiting exon 10 inclusion, RT-qPCR was employed to measure the amount of each isoform using primers specific for 3R and 4R MAPT. A previously reported Vivo-Morpholino ASO (Gene Tools; uses an arginine-rich peptide to facilitate uptake) complementary to the SRE RNA that directs splicing to the 3R isoform was named as tau ASO and served as a positive control.¹⁰ A scrambled ASO served as a negative control. Both 1 (10 μM) and 2 (1.5 μM) reduced the ratio 4R/3R in HeLa cells transfected with WT or DDPAC mini-genes. In cells transfected with the WT mini-gene, the ratio was reduced by 36±2% and 41±3% for 1 and 2, respectively. Likewise in cells transfected with the DDPAC mini-gene, the reduction was 35±5% for 1 and 42±3% for 2 (FIG. 3A). A very small and not statistically significant change (<16%) in the 4R/3R ratio was observed upon treatment of HeLa cells that expressed the WT I17T or DDPAC I17T mutant (lack the A-bulge binding site) while the tau ASO but not scrambled ASO altered the 4R/3R ratio (FIG. 3B). Percent spliced in index (PSI) value indicates how efficiently the exon is spliced into transcripts. PSI₁₀ (PSI of MAPT exon 10) was downregulated by for 1 and for 2 compared to vehicle in LAN5 cells as well as HeLa cells transfected by WT and DDPAC mini-gene but not WT I17T or DDPAC I17T mutant (FIGS. 12A-12C).

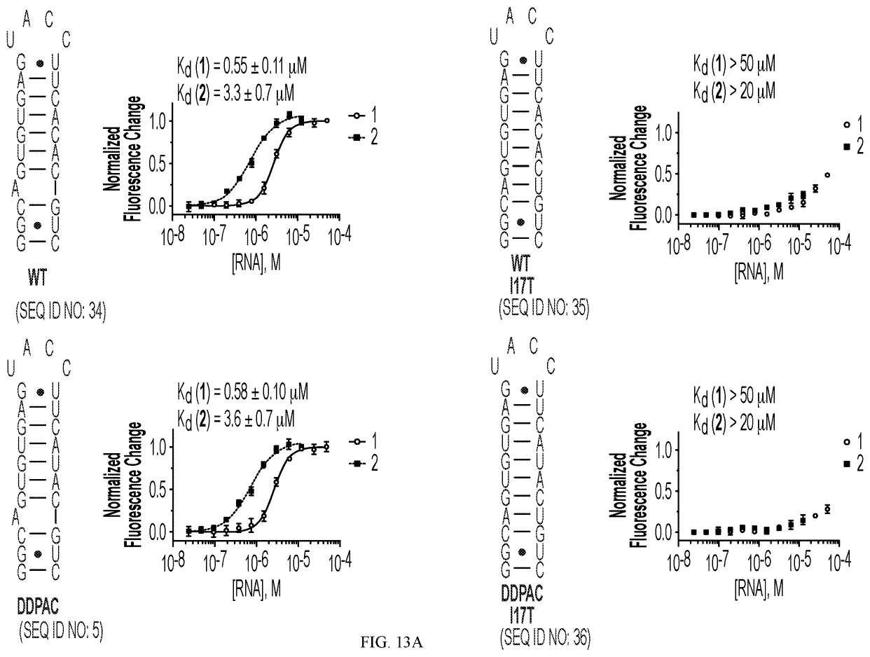

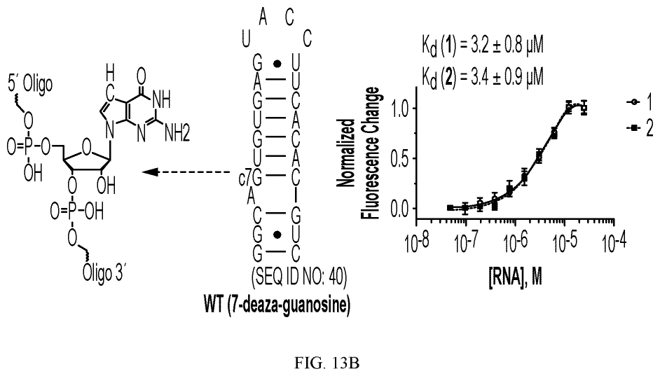

In vitro binding affinity of 2 for the MAPT SRE. The affinities of 1 and 2 for the WT and DDPAC MAPT SRE were measured by monitoring the change in the inherent fluorescence of the small molecules as a function of RNA concentration. Both 1 and 2 bound to the WT RNA, with K_{d}s of 3.3±0.7 μM and 0.55±0.11 μM, respectively, which were similar for the DDPAC RNA (K_{d}=3.6±0.7 μM and 0.58±0.1 μM for 1 and 2, respectively (FIG. 13A). No saturable binding was observed to either the WT I17T or DDPAC I17T mutant; the K_{d} was estimated to be >50 μM (FIG. 13A). Since the interaction of 2 with the MAPT SRE is ~6-fold higher affinity than that for 1, these data suggest that the 2-aminopyridine is indeed forming favorable interactions with the RNA. To investigate whether the 2-aminopyridine module was forming a base triple with the GC pair closing to the A-bulge binding site, the guanosine residue was replaced with 7-deaza-guanosine (Hoogsteen face; hydrogen bonds in a base triple; FIG. 13B) in the WT RNA. The affinity of 2 for the modified RNA decreased from 0.55±0.11 to 3.4±0.9 μM (FIG. 13B). This replacement did not affect the binding affinity of 1, in agreement with its limited interaction with the guanosine in the NMR structure (FIGS. 2C, 13B).

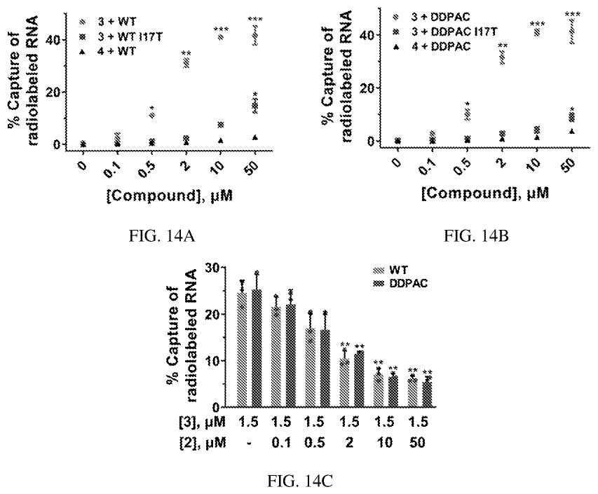

To verify target engagement in vitro, Chemical Cross-Linking and Isolation by Pull-down (Chem-CLIP)²²⁻²⁴ was employed (FIG. 3C). Compound 2 was appended with diazirine and alkyne functionalities to afford Chem-CLIP probe 3 (FIG. 3D); the diazirine forms a cross-link upon irradiation with UV light while the alkyne is used as a chemical handle for pull-down. Compound 4, which lacks the RNA-binding module, was used as a control Chem-CLIP probe (FIG. 3D). The ability of 3 to engage and pull-down radioactively labeled WT and DDPAC SRE RNA was studied, affording similar IC₅₀s, around 1 μM, in agreement with the similar affinity of 2 observed for the two RNAs (FIGS. 14A-14B). The maximum percent pull-down observed for both RNAs was ~40%, at the 10 and 50 μM concentrations (FIGS. 14A-14B). For comparison, the Chem-CLIP probe derived from 1 has a previously reported IC₅₀ around 10 μM.¹⁵ Engagement of the two I17T mutants by 3 was not observed until a concentration of 50 μM (15±0.9% for WT I17T and 9±0.7% for DDPAC I17T; FIGS. 14A-14B). Control Chem-CLIP probe 4 did not engage any of the RNAs tested. Chem-CLIP target validation can also be completed as a competition experiment between the Chem-CLIP probe and the parent compound (C-Chem-CLIP). C-Chem-CLIP experiments revealed that 2 competed the binding of 3 (1.5 μM) for WT and DDPAC SRE RNA similarly with IC₅₀s of ~2 μM, indicating the two compounds bind the same A-bulge binding site (FIG. 14C).

These in vitro target engagement studies were recapitulated in Hela cells transfected with the WT and DDPAC MAPT exon 10 splicing mini-genes. Here, the enrichment of the MAPT reporter was calculated by comparing the amount in the pull-down fractions to the amount in the cell lysate, as normalized to the housekeeping gene GAPDH, by RT-qPCR. Chem-CLIP probe 3 (1.5 μM) enriched the WT and DDPAC reporter by 3.9±0.1-fold and 3.8±0.1-fold, respectively, and in both cases the interaction was competed dose dependently by 2 (FIG. 3E). No pull-down of either reporter was observed by control diazirine probe 4 nor was enrichment observed of the WT I17T or DDPAC I17T mutants by 3 (FIG. 3E). The latter suggests that 2 and 3 engage the A-bulge present in the MAPT SRE.

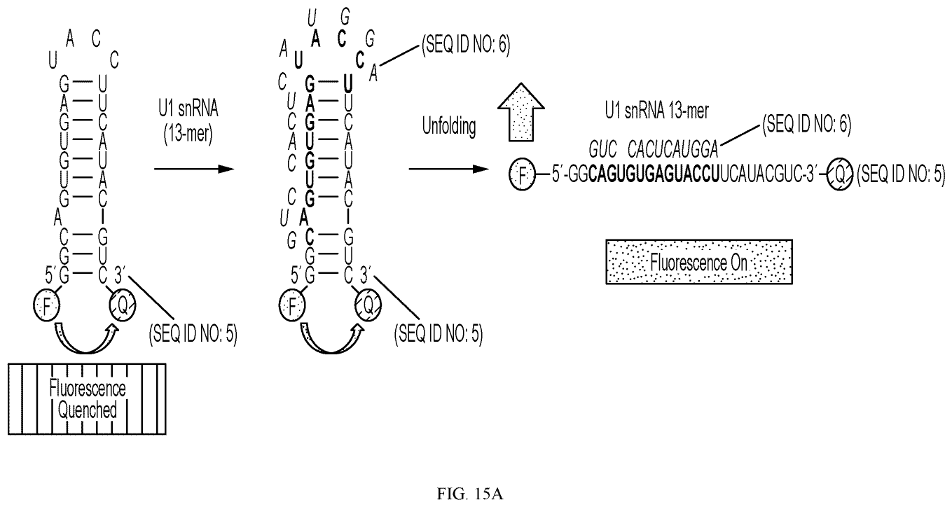

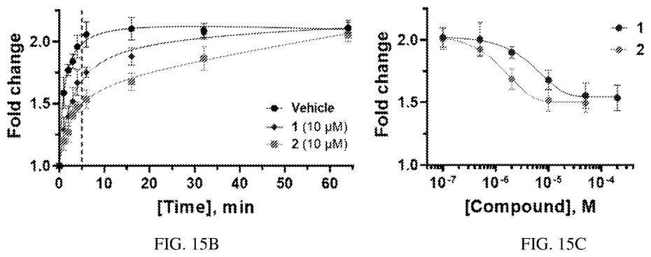

Compounds 1 and 2 reduce U1 small nuclear (sn) RNA binding by thermodynamically stabilizing the MAPT SRE in vitro. An assay was previously reported that assesses a compound's ability to impede the binding of U1 snRNA to the MAPT SRE.¹⁵ A model of the DDPAC RNA hairpin was dually labeled with on the 5′ and 3′ ends with fluorescein (FAM) and a black hole quencher (BHQ), respectively. This RNA can be unfolded by the addition of an oligonucleotide mimic of U1 snRNA, measured by an increase in FAM fluorescence as a function of time (FIG. 15A). The addition of 10 μM of both 1 and 2 resulted in a slower rate in the increase of FAM signal compared to vehicle (0.49 min-1), where the rate of unfolding in the presence of 2 (0.37 min-1) was slower than that for 1 (0.41 min-1, FIG. 15B). Using an end-point measurement (5 min), a dose dependent reduction in FAM fluorescence was observed upon the addition of 1 or 2, affording IC₅₀S of 5.9±1.7 μM and 0.9±0.2 μM, respectively (FIG. 15C). The results from both time- and dose-dependence studies reflect the higher affinity that 2 has for the MAPT SRE than 1 does.

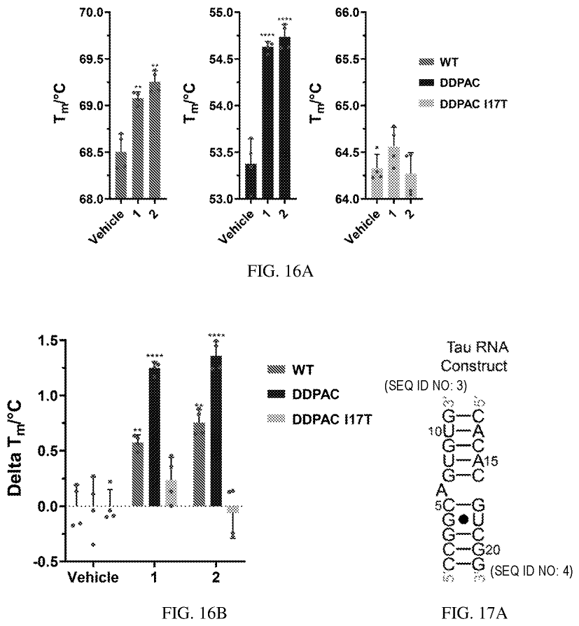

The biochemical assays described above suggest that 1 and 2 thermodynamically stabilize the MAPT SRE RNA hairpin, which was verified using optical melting experiments of the DDPAC, WT, and DDPAC I17T model RNAs in the presence or absence of 1 at 10 μM or 2 at 1.5 μM. Both compounds increased the Tₘ of WT (by 0.58±0.06° C. for 1 and by 0.76±0.1° C. for 2) and DDPAC (by 1.25±0.06° C.) for 1 and by 1.36±0.11° C. for 2) RNAs (FIGS. 16A-16B), where the increase was larger for 2 in agreement with affinity measurements (FIG. 13A-13B). Addition of either compound had no effect on the Tₘ of the DDPAC I17T mutant, which lacks the small molecule binding site (FIGS. 16A-16B).

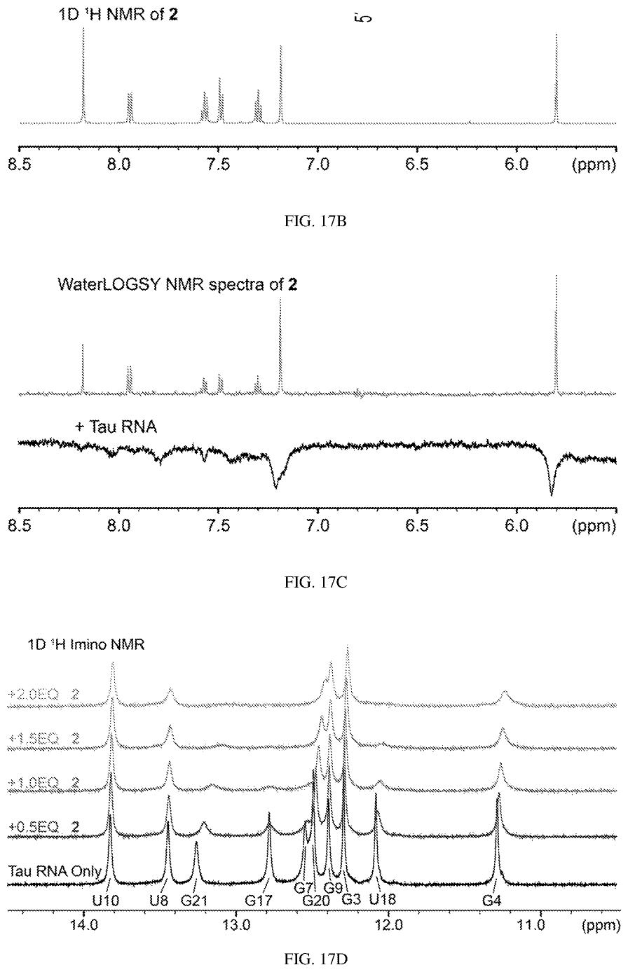

Studying 2-MAPT SRE interactions by NMR spectroscopy and modeling. As further evidence that 2 engages the MAPT DDPAC SRE in vitro, various NMR spectral studies were carried out, including WaterLOGSY³⁰ (water Ligand Observed via Gradient SpectroscopY) and 1D ¹H H₂O NMR spectra. In WaterLOGSY studies with a duplex model of the WT SRE (FIG. 17A), addition of the RNA changed the sign of the NOE, resulting in the appearance of negative peaks (FIGS. 17B-17C). In complementary studies, addition of 2 to the RNA revealed dose dependent changes in the RNA's 1D ¹H spectrum (FIG. 17D). Imino proton resonances for G4 and U18, which form a G·U base nearby by the A-bulge, and G20 shifted up-field upon the addition of 0.5 equivalents of 2; U18 completely disappeared upon addition of 1.5-2.0 equivalents of 2 while G4 broadened from 0.5-2 equivalents. Resonances for G7 and G17, which form the GC base pairs closing to the A-bulge, and G21 were broadened and disappeared as 0.5-1.0 equivalents of 2 were added. Resonances for G3 (slightly), U8 (significantly), G9 [slightly and begins to merge with G7 (significantly) and G20 (significantly)], and U10 (slightly) were also broadened, but otherwise remain unchanged.

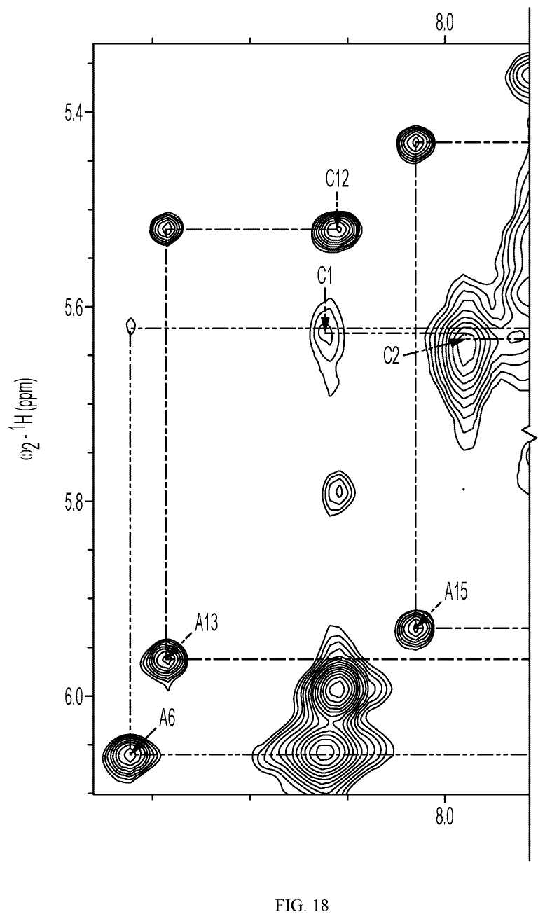

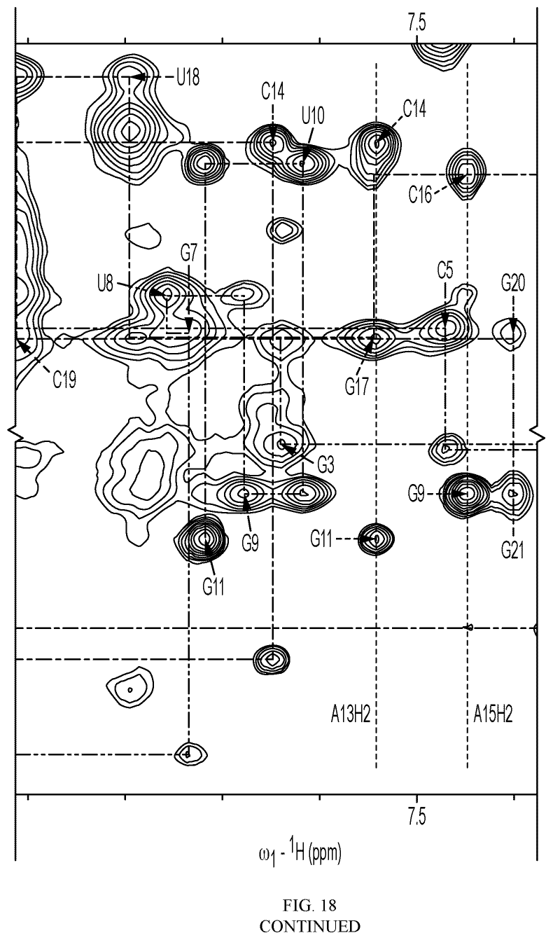

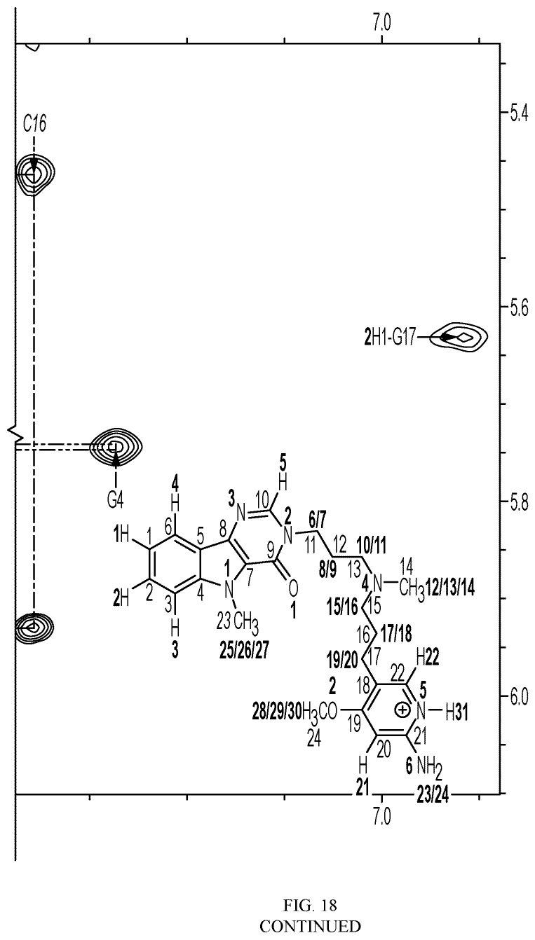

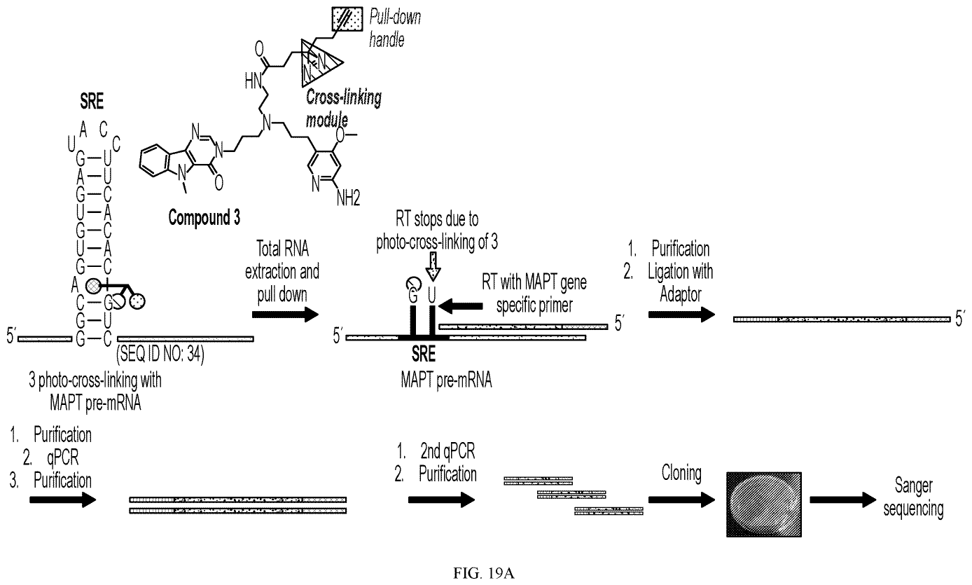

In addition, 2D ¹H-¹H D₂O NMR spectra were acquired to assign nonexchangeable protons. In a 400 ms mixing time NOESY spectrum at 35° C. (FIG. 18), a sequential H6/H8 to H1′ NOESY walk was assigned through all residues except for G20, which could not be assigned due to spectral overlap. A single NOE was observed from H5/H6 of the compound to G17H1′ (forms one of the A-bulge's closing base pairs), which orients the benzene ring within the A bulge site. Overall, NOE patterns the RNA and compound were similar to those observed in 2D NOESY spectra of the Tau WT RNA with 123, but with broader signals likely due to intermediate exchange associated with higher ligand-RNA affinity or complex solubility.

To better understand the orientation adopted by 2 within the binding pocket provided by the A-bulge and a more detailed structural analysis, 2 was docked into the previously elucidated NMR structure of the MAPT WT RNA in complex with 1; 1 was removed to create an apo RNA. Before studying the 2-RNA complex, it was first verified that AutoDock 4.0 (GPU accelerated version) could recapitulate the bound structure of 1. The lowest energy bound state predicted by docking 1 into the MAPT WT RNA overlapped with the solution structure with an RMSD of 0.535 Å. AutoDock-GPU was then used for untargeted and unrestrained docking of 2 against the RNA. The resulting lowest energy state of 2 bound to the RNA yielded an orientation in accordance with the single NOE observed between H5/H6 of the compound to G17H1′ (FIG. 18). A closer inspection of the bound structure revealed that the interaction between 2 and the RNA was stabilized by a combination of stacking interactions with the A-bulge close base pairs, multiple hydrogen bonds (with bases as well as the phosphodiester backbone), and hydrophobic interactions, affording a binding energy of −9.19 kcal/mol (Table 1). This structure was used to perform molecular dynamics (MD) simulations. The resultant clustering of the obtained trajectory and free energy calculations (Table 2) showed that the 2-aminopyridine group forms a base triple with the G7-C₁₆ pair via two hydrogen bonds with the G7 base in the major groove (FIG. 2C).

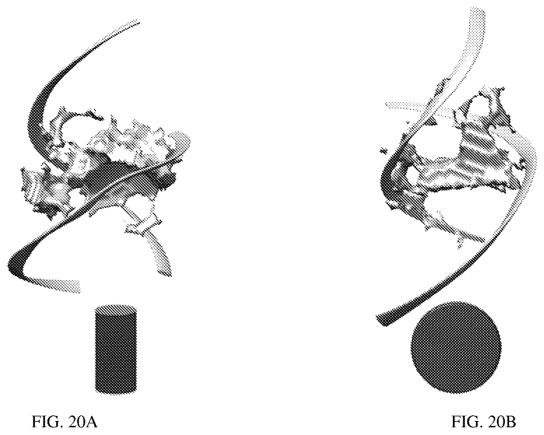

The topology of the binding site when 1 and 2 were bound to the WT MAPT SRE was compared using the concept of principles moment of inertia (PMI). In brief, PMI serves as a descriptor to assess the shape of a given molecule which can be rod-shaped, disc-shaped or sphere-shaped. These calculations showed that the two topologies adopted by 1 and 2 are different, where the binding pocket of 2 adopted a rod-shape topology while 1 adopted a sphere-like topology, providing clues for molecular recognition and possible lead optimization (FIG. 20A-20B). Similar to 1, the ellipticine core of 2 displaced the A-bulge from the co-axial helical axis and stacked on G7 (one of the A-bulge's closing base pairs) and the closing base pair formed by C₅-G17A-bulge. Although 1 stacked on C₅-G17 base pair, it did not form stacking interactions with G7. The H5/H6 proton of 2 lies in the A-bulge, consistent with its observed NOE with G17H1′. Furthermore, the 2-aminopyridine group in 2 forms two hydrogen bonds with the backbone and a hydrogen bond with C14. These additional interactions stabilize the 2-RNA complex more than the 1-RNA complex. These differences are reflected in the binding energies of 1 and 2, −8.91 kcal/mol and −9.19 kcal/mol, respectively. 2 occupied a larger surface area within the binding pocket than 1 (~723.8 Å2 vs. ~330.1 Å2), also contributing to the stronger interaction of 2 with the MAPT RNA.

Compound 2 inhibits endogenous MAPT exon 10 inclusion and generation of 4R tau protein in cells. The biophysical, biochemical, and cellular reporter data suggest that 2 may bind the endogenous MAPT SRE and direct splicing away from the 4R isoform. The effect of 1 on the alternative splicing of exon 10 at the RNA and protein levels was previously studied in the neuroblastoma cell line LAN5. At a 10 UM dose, 1 reduced the 4R/3R ratio of the MAPT RNA isoforms by 44±8% and protein isoforms by 37±4%, 15 In the present disclosure, 2 (1.5 μM) facilitated exon 10 exclusion and reduced the 4R/3R ratio by 56±4%, as determined by RT-qPCR (FIG. 3A), and the 4R/3R protein ratio by 54±9%, as determined by Western blotting (FIG. 3G).

Compound 2 binds endogenous MAPT pre-mRNA in LAN5 cells. Both in vitro Chem-CLIP and Chem-CLIP studies using the MAPT mini-genes in transfected HeLa cells demonstrated that 2 bound the MAPT SRE (FIGS. 3E, 14A-14C). It was studied whether 2, as its activity indicates, binds the SRE in the context of native, endogenous MAPT in LAN5 cells. A 3.6±0.2-fold enrichment of MAPT pre-mRNA was observed upon treatment with 1.5 μM of 3; no enrichment was observed upon treatment with 4 (FIG. 3F). Further, C-Chem-CLIP studies revealed that the cross-linking and hence binding of 3, could be competed by 1 or 2 in dose dependent fashion (FIG. 3F). The concentrations of 1 required to compete 3 were greater than those required by 2 (5 μM of 1 competed 3 to a less extent as 1.5 μM of 2 did).

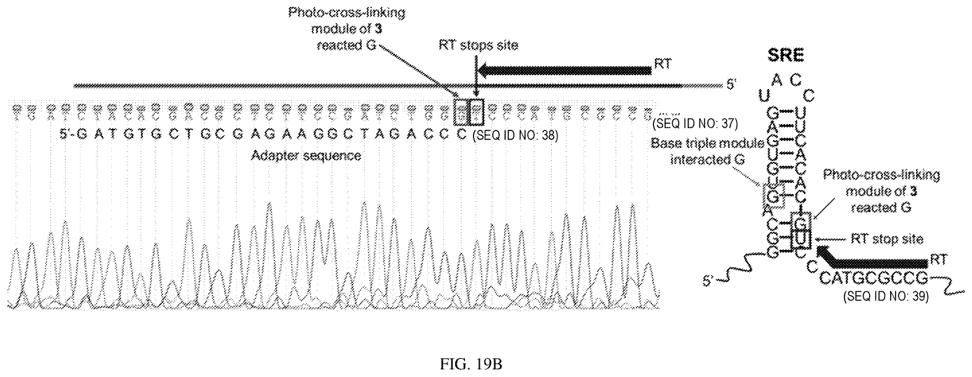

Mapping the binding site of 2 in LAN5 cells using Chem-CLIP-Map-Seq. Although no significant enrichment of the MAPT I17T mutants by 3 indicated selective occupancy of the SRE's A-bulge, the precise binding site of 2 within MAPT pre-mRNA in LAN5 cells was mapped using a method named Chem-CLIP to Map Small Molecule-RNA Binding Sites (Chem-CLIP-Map-Seq).³¹ Cross-linking disallows the processivity of reverse transcriptase, resulting in an “RT stop”. Thus, the nucleotide where truncation of the cDNA occurs indicates the binding site. After the pull-down of MAPT RNA by 3 in LAN5 cells, reverse transcription was completed with a gene-specific primer followed by ligation of single stranded (ss) DNA adaptors and cloning (FIG. 19A). Sequencing analysis showed that cross-linking occurred to the G in the 5′ CG closing base pair of the A-bulge (FIG. 19B). These results are consistent with the formation of base triple as the cross-linking module is positioned on the opposite strand from where 2-aminopyridine forms a base triple (FIGS. 2C, 19B).

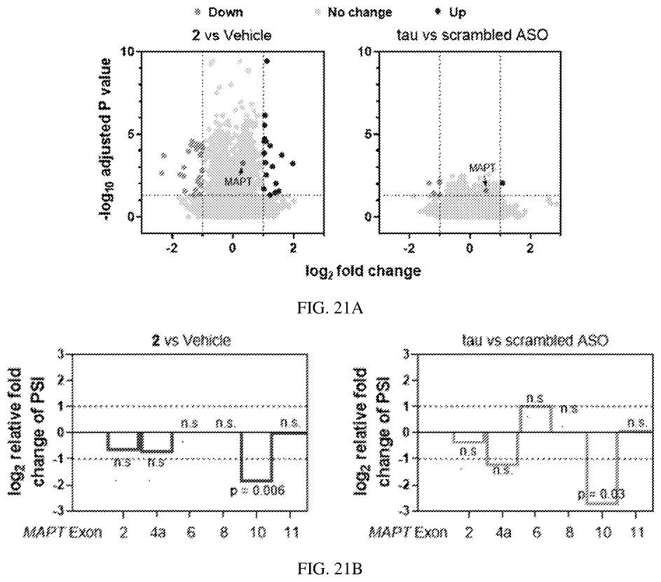

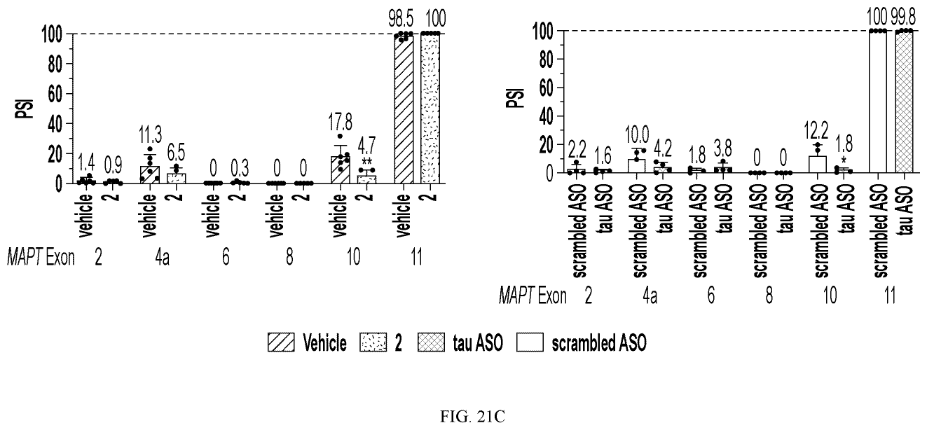

RNA-seq experiment demonstrates 2's selectivity across the transcriptome. RNA-seq analysis in LAN5 cells was completed to investigate whether 2 (1.5 μM; where the 4R/3R MAPT ratio is reduced by ~56%; FIG. 3A) selectively altered the splicing of MAPT exon 10 transcriptome-wide, in comparison to a 4R-to-3R-directing ASO (0.5 μM; 4R/3R MAPT ratio is reduced by ~82%). Differentially expressed genes were identified using quantification and analyses from the Kallisto and Sleuth packages in R. For gene abundance, genes having an absolute fold change>2 with an adjusted P value<0.05 were considered to be significantly altered in expression. Among the 15,404 genes commonly detected in all treated samples, very few changes were observed upon 2 (15,362/15,404 genes were unchanged; 99.73%) or tau ASO treatment (15,399/15,404 genes were unchanged; 99.97%), suggesting limited off-target effects for both 2 and tau ASO (FIG. 21A). Neither 2 nor the ASO altered the expression of MAPT gene (FIG. 21A). Inspection of MAPT alternative splicing showed that the exon 10 among all MAPT exons is the only one significantly reduced in both 2- and tau ASO-treated samples (FIG. 21B-21C). These findings suggested the two modalities function not by changing MAPT abundance or affecting alternative splicing of the other exons but by only inhibiting exon 10 inclusion.

Compound 2 directs exon 10 alternative splicing in primary neurons from htau mice. Next, 2 was evaluated in cultured primary neurons extracted from the cortex of htau transgenic mice. The humanized tau mice lack endogenous murine MAPT gene expression and express all six isoforms (including both 3R and 4R forms) of human MAPT,³² and exhibit age-dependent impairment of cognitive and synaptic function.³³ Brains from htau mice pups were isolated, and the neurons from the cortex were collected and cultured in precoated plastic plates. These neurons require 15 days to express 3R and 4R MAPT RNA at measurable levels. 15 Therefore, after 15 days, the neurons were treated with 1 or 2 as well as the Vivo-Morpholino ASO complementary to the SRE RNA (tau ASO) or scrambled ASO¹⁰ for 48 h. The 4R/3R MAPT ratio was measured by RT-qPCR, revealing dose dependent reduction of the 4R/3R ratio as well as PSI₁₀ by the tau ASO, 1 and 2, where 2 was more potent than 1 (FIGS. 3H, 12C).

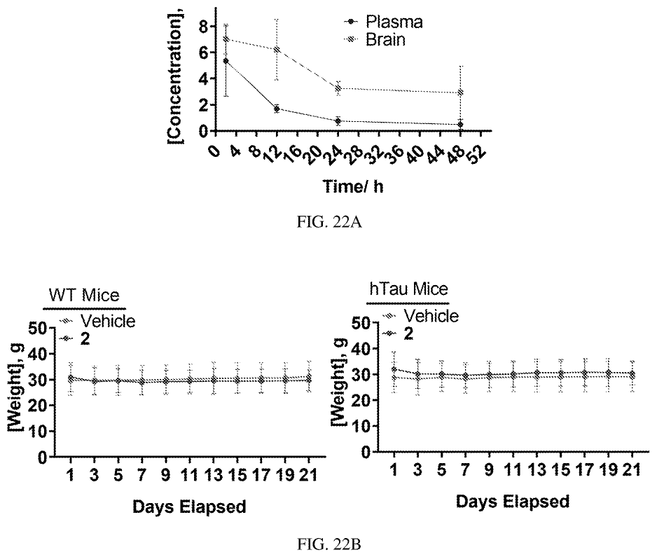

Compound 2 directs exon 10 alternative splicing and alleviates aberrant behavioral phenotypes in htau mice. As described above, htau mice express measurable levels of MAPT isoforms including 3R and 4R tau and its increased expression causes age-dependent behavioral deficits.³³ To determine whether 2 can direct MAPT alternative splicing in vivo, in vitro and in vivo drug metabolism and pharmacokinetics (DMPK) studies were completed. In in vitro studies, equilibrium dialysis was used to assess the free fraction of 2 in plasma, affording a free fraction of 8.9±0.9%. For comparison, the percent free for Ritonavir (a protease inhibitor used for the treatment of HIV) and Carbamazepine (an oral drug used to treat seizures) were 0.18±0.02% and 37.7±4.2%, respectively. Upon oral administration of 100 mg/kg of 2 to C₅₇BL/6J mice, 2.9±1.6 μM was present in brain and 0.5±0.3 μM in plasma 48 h post-administration (FIG. 22A).

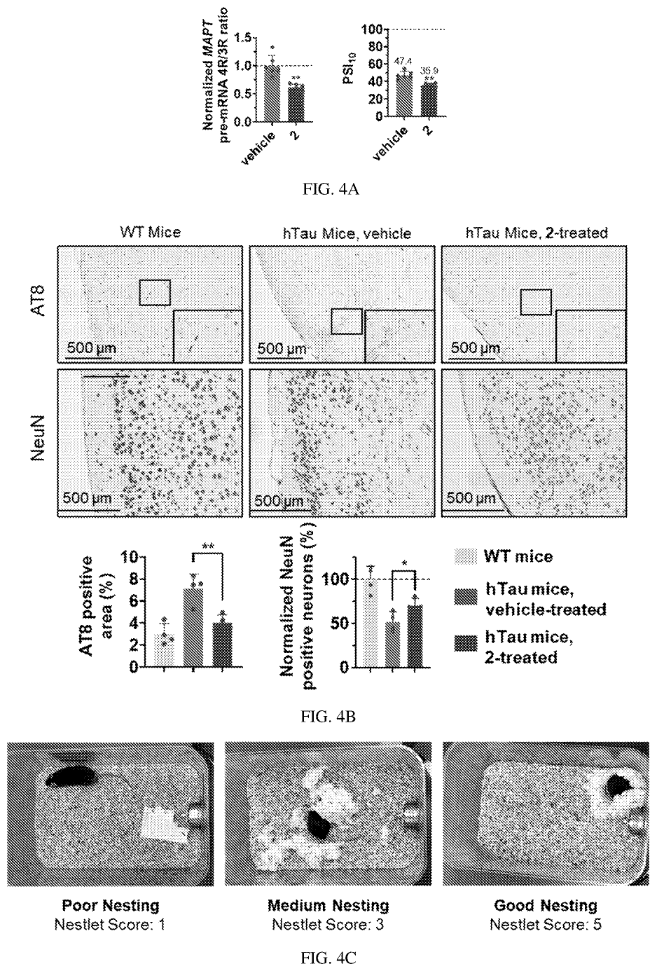

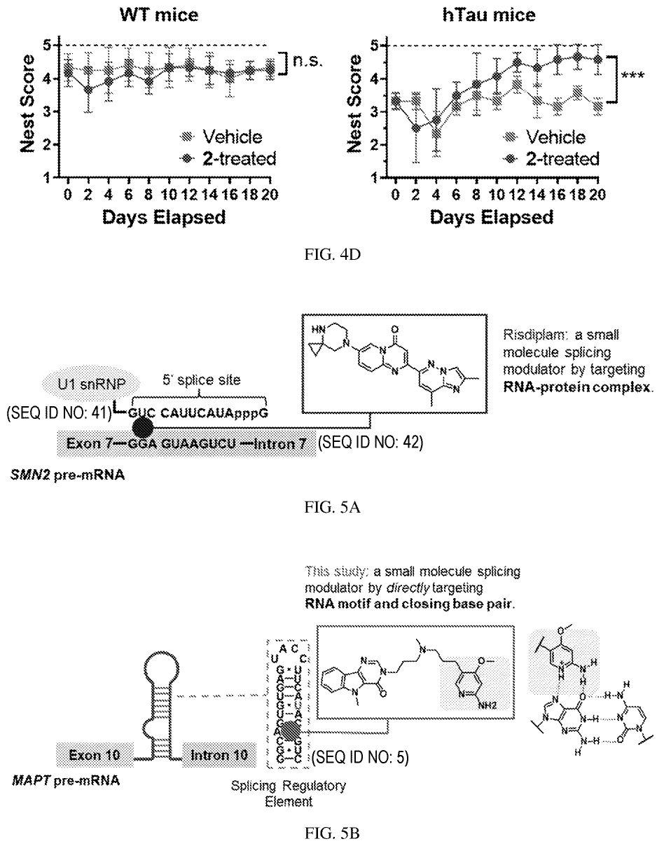

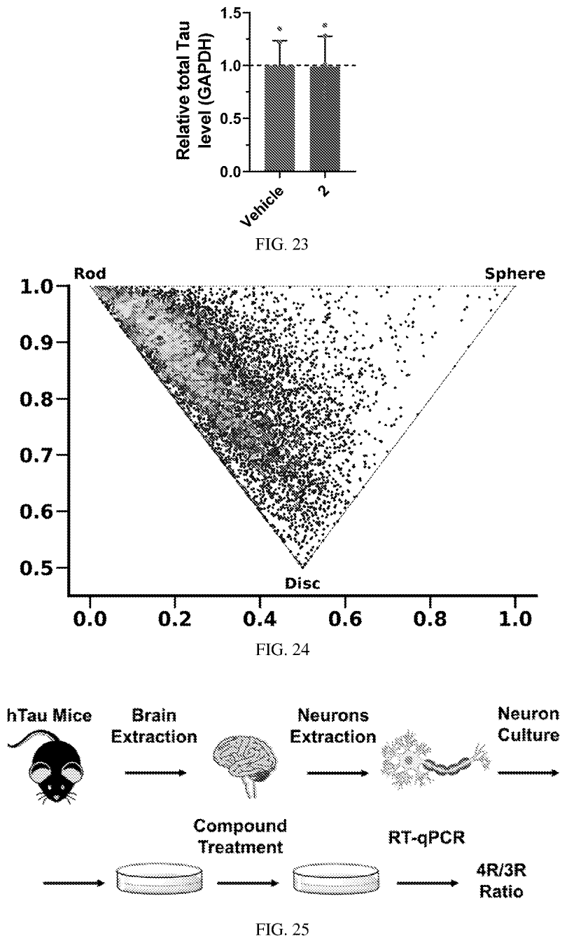

Oral administration of 100 mg/kg of 2 q.o.d. to htau mice was well tolerated as no change in the weight of WT or htau mice was observed throughout the 20-day treatment period (FIG. 22B). After the treatment period, the abundance of 3R and 4R MAPT isoforms was assessed by RT-qPCR, where a significant reduction of the 4R/3R ratio (38±5%) and PSI₁₀ (12±2%) was observed without alteration of total human MAPT transcript levels (FIGS. 4A, 23).

Aberrant tau protein phosphorylation is frequently associated with tauopathies caused by imbalanced 4R/3R ratio 34-35 and can be observed in the htau mouse model.³⁵ Thus, sagittal brain sections were assessed for tau phosphorylation by immunohistochemistry (IHC) using AT8 antibody which detects S202/T205 phosphorylation events.³⁴ Treatment of 2 reduced antibody immunoreactivity throughout the cortex, indicative of decreased pathological tau burden with a lower 4R/3R ratio (FIG. 4B). NeuN, a neuron-specific protein, was used as a marker to quantify neuron viability, which was enhanced in 2-treated mice (FIG. 4B).

One of behavioral deficits observed in htau mice is impaired nesting behavior,³⁵⁻³⁶ which can be quantified based on published criteria (FIG. 4C).³⁷ From Day 1 to Day 20, mice were individually housed and nesting material from the previous day was removed. An intact 3.0 g nestlet was placed within each cage. Around 15 h later, the resulting nest was photographed for classification, and the untorn nestlet was weighed if there were any. Nesting is scored on a rating scale of 1-5³⁷ where if the nestlet is over 90% intact, the nestlet score is 1 (Poor; FIG. 4C). When the nestlet is partially torn but >50% remains, the nestlet score is 2; a score of 3 is assigned when 50-90% of the nestlet is torn but spread around the cage and no identifiable nest site is found (Medium; FIG. 4C). When more than 90% of the nestlet is torn and the nest is identifiable but flat, the nestlet score is 4. When a near perfect nest is built and the wall is higher than the mouse's body, it is assigned a nestlet score of 5 (Good; FIG. 4C).

WT mice showed no significant difference in nesting behavior without or with 2-treatment after 20 days, and the average nest score was consistent over time, ranging from 4.0-4.4 and 3.7-4.3, respectively. On average, the nest score at Day-20 for untreated and 2-treated WT mice was 4.3±0.1 and 4.1±0.2, respectively (FIG. 4D). The average nesting score for untreated htau mice was consistently below 3.8 and was 3.2±0.2 after 20 days. In contrast, the nesting scores for 2-treated mice improved over the treatment period, on average 2.6±1.1 at Day 2 and 4.5±0.4 at Day 20 (FIG. 4D). Collectively, these in vivo studies demonstrated that 2 is orally bioavailable, well tolerated, and able to mitigate cellular and behavioral phenotypes observed in htau mice.

Other modalities, including ASOs and small molecules, have been discovered that direct alternative pre-mRNA splicing.³⁸⁻⁴² Indeed, two have been approved for the treatment of spinal muscular atrophy (SMA), a genetic disease caused by the deletion or mutation of the survival motor neuron 1 (SMN1) gene. This gene encodes for a catalytic component of a complex that assembles snRNPs that comprise the spliceosome. The loss of function in SMA causes degradation of spinal motor neurons that results in muscle weakness and atrophy as well as other complications. Humans encode an SMN1 paralogue, SMN2, that differs by two nucleotides, one sequence in exon 7 and the other in exon 8. The former disrupts a splicing enhancer, resulting in the exclusion of SMN2 exon 7 and reduced half-life of the encoded protein, relative to WT SMN1. Therefore, if the SMN2 pre-mRNA splicing outcome could be directed to include exon 7, then SMN2 could substitute functionally for the loss of SMN1 and hence as a treatment for SMA.⁴³⁻⁴⁴

The first approved modalities were oligonucleotide-based medicines such as nusinersen (Spinraza) that affect SMN pre-mRNA splicing by targeting an intronic splicing silencer downstream of the 5′ splice site of exon 7.⁴¹⁻⁴² Small molecules were discovered from phenotypic screening and later optimized, including risdiplam (FIG. 5A). It was later determined that risdiplam functions as a molecular glue to stabilize an RNA helix formed by the 5′ splice site of SMN2 exon 7 and 5′ terminus of U1 snRNA/U1 snRNP (FIG. 5A).⁴⁵⁻⁴⁷

Therefore, the compound has a complex mode of action that includes the stabilization of an RNA-macromolecular complex including protein. In the present disclosure, 2 has a simpler mode of action where it binds the MAPT SRE and thermally stabilizes its structure to impede U1 snRNA binding as designed (FIG. 5B).

The design principles outlined in the present disclosure emphasize the importance of shape complementarity of the binding pocket beyond the scope of the topological properties as an approach to increase the favorable interactions and hence potency of a small molecule. RNA-binding pockets are quite different than protein-binding pockets and thus requires new design strategies that address both affinity and specificity.⁴⁰ The molecule designed herein delineates a new structure-based drug design approach exploiting the spatial proximity to extend the binding pocket beyond what is usually perceived as a binding pocket. In particular, it is shown herein that T-shaped small molecules, grown vertically, can have sub-micromolar binding affinity. Thus T-shaped geomorphic compound design principles could be a general strategy to generate small molecules targeting RNA with favorable physicochemical properties, bio-distribution, and oral bioavailability as exemplified in the present disclosure. The shape distribution of compounds in DrugBank has a high enrichment of the rod-like molecules with thinner distribution for disk-like or sphere-like molecules (FIG. 24). Very few T-shaped molecules are observed, which is a restraint imposed by the binding pocket topology of proteins which prevents longitudinal growth. This suggests that a T-shape topology could be a general strategy for targeting RNAs specifically over proteins.

Targeting RNAs using a base-triple-formation strategy is an area of ongoing investigation in the peptide-nucleic acid space.^{21, 48-51} Emerging datasets described here could provide additional modules that could be appended to small molecule RNA binders as a means to increase in affinity and specificity. This base-triple-formation strategy could be applied to optimize compounds that target other incurable diseases caused by aberrant pre-mRNA splicing.

### REFERENCES

1. McManus, C. J.; Graveley, B. R., RNA structure and the mechanisms of alternative splicing. Curr Opin Genet Dev 2011, 21 (4), 373-9.
2. Chen, M.; Manley, J. L., Mechanisms of alternative splicing regulation: insights from molecular and genomics approaches. Nat Rev Mol Cell Biol 2009, 10 (11), 741-54.
3. Tazi, J.; Bakkour, N.; Stamm, S., Alternative splicing and disease. Biochim Biophys Acta 2009, 1792 (1), 14-26.
4. Cieply, B.; Carstens, R. P., Functional roles of alternative splicing factors in human disease. Wiley Interdiscip Rev RNA 2015, 6 (3), 311-26.
5 Ingram, E. M.; Spillantini, M. G., Tau gene mutations: dissecting the pathogenesis of FTDP-17. Trends Mol Med 2002, 8 (12), 555-62.
6. Goedert, M.; Spillantini, M. G.; Jakes, R.; Rutherford, D.; Crowther, R. A., Multiple isoforms of human microtubule-associated protein tau: sequences and localization in neurofibrillary tangles of Alzheimer's disease. Neuron 1989, 3 (4), 519-26.
7 Goedert, M.; Jakes, R., Expression of separate isoforms of human tau protein: correlation with the tau pattern in brain and effects on tubulin polymerization. EMBO J 1990, 9 (13), 4225-30.
8. Hong, M.; Zhukareva, V.; Vogelsberg-Ragaglia, V.; Wszolek, Z.; Reed, L.; Miller, B. I.; Geschwind, D. H.; Bird, T. D.; McKeel, D.; Goate, A.; Morris, J. C.; Wilhelmsen, K. C.; Schellenberg, G. D.; Trojanowski, J. Q.; Lee, V. M., Mutation-specific functional impairments in distinct tau isoforms of hereditary FTDP-17. Science 1998, 282 (5395), 1914-7.
9. Brunden, K. R.; Trojanowski, J. Q.; Lee, V. M., Advances in tau-focused drug discovery for Alzheimer's disease and related tauopathies. Nat Rev Drug Discov 2009, 8 (10), 783-93.
10. Donahue, C. P.; Muratore, C.; Wu, J. Y.; Kosik, K. S.; Wolfe, M. S., Stabilization of the tau exon 10 stem loop alters pre-mRNA splicing. The Journal of biological chemistry 2006, 281 (33), 23302-6.
11. Lynch, T.; Sano, M.; Marder, K. S.; Bell, K. L.; Foster, N. L.; Defendini, R. F.; Sima, A. A.; Keohane, C.; Nygaard, T. G.; Fahn, S.; et al., Clinical characteristics of a family with chromosome 17-linked disinhibition-dementia-parkinsonism-amyotrophy complex. Neurology 1994, 44 (10), 1878-84.
12. Grover, A.; Houlden, H.; Baker, M.; Adamson, J.; Lewis, J.; Prihar, G.; Pickering-Brown, S.; Duff, K.; Hutton, M., 5′ splice site mutations in tau associated with the inherited dementia FTDP-17 affect a stem-loop structure that regulates alternative splicing of exon 10. J Biol Chem 1999, 274 (21), 15134-43.
13. Zheng, S.; Chen, Y.; Donahue, C. P.; Wolfe, M. S.; Varani, G., Structural basis for stabilization of the tau pre-mRNA splicing regulatory element by novantrone (mitoxantrone). Chem Biol 2009, 16 (5), 557-66.
14. Varani, L.; Spillantini, M. G.; Goedert, M.; Varani, G., Structural basis for recognition of the RNA major groove in the tau exon 10 splicing regulatory element by aminoglycoside antibiotics. Nucleic Acids Res 2000, 28 (3), 710-9.
15. Chen, J. L.; Zhang, P.; Abe, M.; Aikawa, H.; Zhang, L.; Frank, A. J.; Zembryski, T.; Hubbs, C.; Park, H.; Withka, J.; Steppan, C.; Rogers, L.; Cabral, S.; Pettersson, M.; Wager, T. T.; Fountain, M. A.; Rumbaugh, G.; Childs-Disney, J. L.; Disney, M. D., Design, Optimization, and Study of Small Molecules That Target Tau Pre-mRNA and Affect Splicing. J Am Chem Soc 2020, 142 (19), 8706-8727.
16. Angelbello, A. J.; Rzuczek, S. G.; McKee, K. K.; Chen, J. L.; Olafson, H.; Cameron, M. D.; Moss, W. N.; Wang, E. T.; Disney, M. D., Precise small-molecule cleavage of an r (CUG) repeat expansion in a myotonic dystrophy mouse model. Proc Natl Acad Sci USA 2019, 116 (16), 7799-7804.
17. Bush, J. A.; Aikawa, H.; Fuerst, R.; Li, Y.; Ursu, A.; Meyer, S. M.; Benhamou, R. I.; Chen, J. L.; Khan, T.; Wagner-Griffin, S.; Van Meter, M. J.; Tong, Y.; Olafson, H.; McKee, K. K.; Childs-Disney, J. L.; Gendron, T. F.; Zhang, Y.; Coyne, A. N.; Wang, E. T.; Yildirim, I.; Wang, K. W.; Petrucelli, L.; Rothstein, J. D.; Disney, M. D., Ribonuclease recruitment using a small molecule reduced c9ALS/FTD r(G(4)C(2)) repeat expansion in vitro and in vivo ALS models. Science translational medicine 2021, 13 (617), eabd5991.
18. Rzuczek, S. G.; Colgan, L. A.; Nakai, Y.; Cameron, M. D.; Furling, D.; Yasuda, R.; Disney, M. D., Precise small-molecule recognition of a toxic CUG RNA repeat expansion. Nat Chem Biol 2017, 13 (2), 188-193.
19. Warf, M. B.; Nakamori, M.; Matthys, C. M.; Thornton, C. A.; Berglund, J. A., Pentamidine reverses the splicing defects associated with myotonic dystrophy. Proc Natl Acad Sci USA 2009, 106 (44), 18551-6.
20. Li, J.; Nakamori, M.; Matsumoto, J.; Murata, A.; Dohno, C.; Kiliszek, A.; Taylor, K.; Sobczak, K.; Nakatani, K., A Dimeric 2,9-Diamino-1,10-phenanthroline Derivative Improves Alternative Splicing in Myotonic Dystrophy Type 1 Cell and Mouse Models. Chemistry 2018, 24 (68), 18115-18122.
21. Zengeya, T.; Gupta, P.; Rozners, E., Triple-helical recognition of RNA using 2-aminopyridine-modified PNA at physiologically relevant conditions. Angew Chem Int Ed Engl 2012, 51 (50), 12593-6.
22. Costales, M. G.; Haga, C. L.; Velagapudi, S. P.; Childs-Disney, J. L.; Phinney, D. G.; Disney, M. D., Small molecule inhibition of microRNA-210 reprograms an oncogenic hypoxic circuit. J Am Chem Soc 2017, 139 (9), 3446-3455.
23. Childs-Disney, J. L.; Disney, M. D., Approaches to validate and manipulate RNA targets with small molecules in cells. Annual review of pharmacology and toxicology 2016, 56, 123-40.
24. Wang, J.; Schultz, P. G.; Johnson, K. A., Mechanistic studies of a small-molecule modulator of SMN2 splicing. Proc Natl Acad Sci USA 2018, 115 (20), E4604-e4612.
25. Berman, H. M.; Westbrook, J.; Feng, Z.; Gilliland, G.; Bhat, T. N.; Weissig, H.; Shindyalov, I. N.; Bourne, P. E., The Protein Data Bank. Nucleic acids research 2000, 28 (1), 235-42.
26. Wager, T. T.; Hou, X.; Verhoest, P. R.; Villalobos, A., Moving beyond rules: the development of a central nervous system multiparameter optimization (CNS MPO) approach to enable alignment of druglike properties. ACS chemical neuroscience 2010, 1 (6), 435-49.
27. Abu Almakarem, A. S.; Petrov, A. I.; Stombaugh, J.; Zirbel, C. L.; Leontis, N. B., Comprehensive survey and geometric classification of base triples in RNA structures. Nucleic Acids Res 2012, 40 (4), 1407-23.
28. Houck-Loomis, B.; Durney, M. A.; Salguero, C.; Shankar, N.; Nagle, J. M.; Goff, S. P.; D'Souza, V. M., An equilibrium-dependent retroviral mRNA switch regulates translational recoding. Nature 2011, 480 (7378), 561-4.
29. Wilusz, J. E.; JnBaptiste, C. K.; Lu, L. Y.; Kuhn, C.-D.; Joshua-Tor, L.; Sharp, P. A., A triple helix stabilizes the 3′ ends of long noncoding RNAs that lack poly (A) tails. Genes & development 2012, 26 (21), 2392-2407.
30. Dalvit, C.; Pevarello, P.; Tato, M.; Veronesi, M.; Vulpetti, A.; Sundstrom, M., Identification of compounds with binding affinity to proteins via magnetization transfer from bulk water. J Biomol NMR 2000, 18 (1), 65-8.
31. Velagapudi, S. P.; Li, Y.; Disney, M. D., A cross-linking approach to map small molecule-RNA binding sites in cells. Bioorganic & medicinal chemistry letters 2019, 29 (12), 1532-1536.
32. Tucker, K. L.; Meyer, M.; Barde, Y. A., Neurotrophins are required for nerve growth during development. Nat Neurosci 2001, 4 (1), 29-37.
33. Polydoro, M.; Acker, C. M.; Duff, K.; Castillo, P. E.; Davies, P., Age-dependent impairment of cognitive and synaptic function in the htau mouse model of tau pathology. The Journal of neuroscience: the official journal of the Society for Neuroscience 2009, 29 (34), 10741-9.
34. Braak, H.; Braak, E., Staging of Alzheimer's disease-related neurofibrillary changes. Neurobiol Aging 1995, 16 (3), 271-8; discussion 278-84.
35. Schoch, K. M.; DeVos, S. L.; Miller, R. L.; Chun, S. J.; Norrbom, M.; Wozniak, D. F.; Dawson, H. N.; Bennett, C. F.; Rigo, F.; Miller, T. M., Increased 4R-Tau induces pathological changes in a human-Tau mouse model. Neuron 2016, 90 (5), 941-7.
36. Maeda, S.; Djukic, B.; Taneja, P.; Yu, G. Q.; Lo, I.; Davis, A.; Craft, R.; Guo, W.; Wang, X.; Kim, D.; Ponnusamy, R.; Gill, T. M.; Masliah, E.; Mucke, L., Expression of A152T human tau causes age-dependent neuronal dysfunction and loss in transgenic mice. EMBO Rep 2016, 17 (4), 530-51.
37. Deacon, R. M., Assessing nest building in mice. Nat Protoc 2006, 1 (3), 1117-9.
38. Kole, R.; Krainer, A. R.; Altman, S., RNA therapeutics: beyond RNA interference and antisense oligonucleotides. Nat Rev Drug Discov 2012, 11 (2), 125-40.
39. Havens, M. A.; Hastings, M. L., Splice-switching antisense oligonucleotides as therapeutic drugs. Nucleic Acids Res 2016, 44 (14), 6549-63.
40. Childs-Disney, J. L.; Yang, X.; Gibaut, Q. M. R.; Tong, Y.; Batey, R. T.; Disney, M. D., Targeting RNA structures with small molecules. Nat Rev Drug Discov 2022, 21 (10), 736-762.
41. Finkel, R. S.; Chiriboga, C. A.; Vajsar, J.; Day, J. W.; Montes, J.; De Vivo, D. C.; Yamashita, M.; Rigo, F.; Hung, G.; Schneider, E.; Norris, D. A.; Xia, S.; Bennett, C. F.; Bishop, K. M., Treatment of infantile-onset spinal muscular atrophy with nusinersen: a phase 2, open-label, dose-escalation study. Lancet 2016, 388 (10063), 3017-3026.
42. Chiriboga, C. A.; Swoboda, K. J.; Darras, B. T.; Iannaccone, S. T.; Montes, J.; De Vivo, D. C.; Norris, D. A.; Bennett, C. F.; Bishop, K. M., Results from a phase 1 study of nusinersen (ISIS-SMN(Rx)) in children with spinal muscular atrophy. Neurology 2016, 86 (10), 890-7.
43. Lunn, M. R.; Wang, C. H., Spinal muscular atrophy. The Lancet 2008, 371 (9630), 2120-2133.
44. Kolb, S. J.; Kissel, J. T., Spinal muscular atrophy: a timely review. Archives of neurology 2011, 68 (8), 979-984.
45. Naryshkin, N. A.; Weetall, M.; Dakka, A.; Narasimhan, J.; Zhao, X.; Feng, Z.; Ling, K. K.; Karp, G. M.; Qi, H.; Woll, M. G.; Chen, G.; Zhang, N.; Gabbeta, V.; Vazirani, P.; Bhattacharyya, A.; Furia, B.; Risher, N.; Sheedy, J.; Kong, R.; Ma, J.; Turpoff, A.; Lee, C. S.; Zhang, X.; Moon, Y. C.; Trifillis, P.; Welch, E. M.; Colacino, J. M.; Babiak, J.; Almstead, N. G.; Peltz, S. W.; Eng, L. A.; Chen, K. S.; Mull, J. L.; Lynes, M. S.; Rubin, L. L.; Fontoura, P.; Santarelli, L.; Haehnke, D.; McCarthy, K. D.; Schmucki, R.; Ebeling, M.; Sivaramakrishnan, M.; Ko, C. P.; Paushkin, S. V.; Ratni, H.; Gerlach, I.; Ghosh, A.; Metzger, F., Motor neuron disease. SMN2 splicing modifiers improve motor function and longevity in mice with spinal muscular atrophy. Science 2014, 345 (6197), 688-93.
46. Palacino, J.; Swalley, S. E.; Song, C.; Cheung, A. K.; Shu, L.; Zhang, X.; Van Hoosear, M.; Shin, Y.; Chin, D. N.; Keller, C. G.; Beibel, M.; Renaud, N. A.; Smith, T. M.; Salcius, M.; Shi, X.; Hild, M.; Servais, R.; Jain, M.; Deng, L.; Bullock, C.; Mclellan, M.; Schuierer, S.; Murphy, L.; Blommers, M. J.; Blaustein, C.; Berenshteyn, F.; Lacoste, A.; Thomas, J. R.; Roma, G.; Michaud, G. A.; Tseng, B. S.; Porter, J. A.; Myer, V. E.; Tallarico, J. A.; Hamann, L. G.; Curtis, D.; Fishman, M. C.; Dietrich, W. F.; Dales, N. A.; Sivasankaran, R., SMN2 splice modulators enhance U1-pre-mRNA association and rescue SMA mice. Nat Chem Biol 2015, 11 (7), 511-7.
47. Ratni, H.; Ebeling, M.; Baird, J.; Bendels, S.; Bylund, J.; Chen, K. S.; Denk, N.; Feng, Z.; Green, L.; Guerard, M.; Jablonski, P.; Jacobsen, B.; Khwaja, O.; Kletzl, H.; Ko, C. P.; Kustermann, S.; Marquet, A.; Metzger, F.; Mueller, B.; Naryshkin, N. A.; Paushkin, S. V.; Pinard, E.; Poirier, A.; Reutlinger, M.; Weetall, M.; Zeller, A.; Zhao, X.; Mueller, L., Discovery of Risdiplam, a Selective Survival of Motor Neuron-2 (SMN2) Gene Splicing Modifier for the Treatment of Spinal Muscular Atrophy (SMA). J Med Chem 2018, 61 (15), 6501-6517.
48. Nielsen, P. E.; Egholm, M.; Berg, R. H.; Buchardt, O., Sequence-selective recognition of DNA by strand displacement with a thymine-substituted polyamide. Science 1991, 254 (5037), 1497-500.
49. Egholm, M.; Buchardt, O.; Christensen, L.; Behrens, C.; Freier, S. M.; Driver, D. A.; Berg, R. H.; Kim, S. K.; Norden, B.; Nielsen, P. E., PNA hybridizes to complementary oligonucleotides obeying the Watson-Crick hydrogen-bonding rules. Nature 1993, 365 (6446), 566-8.
50. Li, M.; Zengeya, T.; Rozners, E., Short peptide nucleic acids bind strongly to homopurine tract of double helical RNA at pH 5.5. J Am Chem Soc 2010, 132 (25), 8676-81.
51. Devi, G.; Yuan, Z.; Lu, Y.; Zhao, Y.; Chen, G., Incorporation of thio-pseudoisocytosine into triplex-forming peptide nucleic acids for enhanced recognition of RNA duplexes. Nucleic Acids Res 2014, 42 (6), 4008-18.

### Example 2: Materials and Methods

General Nucleic Acids Methods. All DNA oligonucleotides used in these studies were purchased from Integrated DNA Technologies, Inc. (IDT). When received, they were dissolved in nanopure water and used without further purification. A Vivo-Morpholino antisense oligonucleotide (ASO) targeting the MAPT exon-10-intron junction hairpin (4R-to-3R tau ASO: 5′-TGAAGGTACTCACACTGCCGC-3′ (SEQ ID NO: 1)) and a scrambled ASO (5′-CTGTCTGACGTTCTTTGT-3′ (SEQ ID NO: 2)) were purchased from Gene Tools, LLC. The stock solution was obtained by dissolving the ASO in water at 1 mM and was stored at 4° C. as recommended by the manufacturer. All RNA constructs were purchased from GE Healthcare Dharmacon, Inc. When received, they were deprotected according to the manufacturer's recommended protocol and desalted with PD-10 columns (GE Healthcare) also per the manufacturer's recommended protocol. RNA oligonucleotide concentrations were determined by their absorbance at 260 nm at 90° C. using a Beckman Coulter DU800 UV/vis spectrophotometer and the corresponding extinction coefficient provided by the manufacturer.

Compounds. Compounds 1 to 4 were synthesized as described in Example 3.

Preparation of NMR Samples. Two duplex-forming oligoribonucleotides r (5′-CCGGCAGUGUG-3′ (SEQ ID NO: 3))+r(5′CACACGUCGG-3′ (SEQ ID NO: 4)) were designed to mimic the stem of the tau wild type (WT) hairpin. Individual RNA oligonucleotides were combined in 400 μL of NMR Buffer [10 mM KH₂PO₄/K₂HPO₄, pH 6.0 and 0.5 mM EDTA] in H₂O or 100% D₂O with a final RNA duplex concentration of 15 μM for WaterLOGSY experiments, 100 μM for 1D 1400 μM for 2D ¹D₂O was added to 5% (v/v) to provide a lock signal. RNA samples were annealed by heating to 95° C. for 3 min, followed by slow cooling to room temperature before being added to Shigemi NMR tubes (Shigemi, Inc.). Compound of interest was added to a final compound: RNA molar ratio of 20.0 for WaterLOGSY experiments, 2.0 for 1D ¹H H₂O experiments, and 1.5 for 2D ¹NOESY experiments.

NMR Spectroscopy. NMR spectra were acquired on Bruker Avance III 600 and 700 MHz spectrometers equipped with cryoprobes. WaterLOGSY spectra were acquired at 298 K, 1D NMR spectra of samples in 95% H₂O/5% D₂O were acquired at 283 K, and 2D NOESY spectra in 100% D₂O were acquired at 308 K. For 1D spectra of samples in 95% H₂O/5% D₂O, an excitation sculpting sequence during acquisition suppressed the water signal. Proton chemical shifts were referenced internally to the frequency of water. WaterLOGSY and 1D ¹H spectra were processed using Bruker TopSpin and 2D NMR spectra were processed with NMRPipe¹ and assigned with SPARKY².

Simulation Protocols. Simulations were carried out with the AMBER 18 simulation package³ using the PARM99 force field⁴ with revised χ⁵ and α/γ⁶ torsional parameters. Each system was first neutralized with Na⁺ ions,⁷ which then was solvated with TIP3P⁸ water molecules in a truncated octahedral box with periodic boundary conditions extended to 10 Å using the LEAP module of Amber 16⁴. Each system was then added with 13 Na⁺ and Cl⁻ ions to mimic physiological conditions, where after equilibration each system had 0.16 M Na+ concentrations. The structures were minimized with the sander module³, each in two steps. A positional restraint (10 kcal mol⁻¹ Å⁻²) was applied to the tau RNA in the first step of minimization with 5000 steps of the steepest-descent algorithm and subsequently followed with the second round of minimization with 5000 steps of the conjugate-gradient algorithm and no restraints. Minimization was followed by an equilibration protocol first in constant volume with restraints on the RNA molecule (10 kcal mol⁻¹ Å⁻²) and gradually increasing the temperature up to 300 K for several nanoseconds using the Langevin thermostat.⁹ A second round of equilibration was performed at constant pressure (1 atm) with the constant temperature at 300 K, and pressure coupling¹⁰ of 1.0 ps⁻¹ gradually removing the constraints on the solute. After minimization and equilibration, a 3 usec MD simulation with a 2 psec time step was performed using NPT (constant temperature and constant pressure) dynamics with isotropic positional scaling. The reference pressure was set to 1 atm with a pressure relaxation time of 2 psec. SHAKE¹¹ was turned on for constraining bonds involving hydrogen atoms. An atom-based long-range cutoff of 8.0 Å was used in the production run. The reference temperature was set to 300 K. Particle Mesh Ewald (PME) was used to handle the electrostatics¹² and the Langevin thermostat¹³ was applied with a coupling constant γ=1.0 ps⁻¹. Simulations were performed using the PMEMD.CUDA implementation of AMBER18.¹²

Parametrization of compound 2. A hand-built model of 2 was geometry-optimized at the HF/6-31G* quantum mechanical (QM) level consistent with AMBER ff10 force fields. Atomic charges were determined using the restrained electrostatic potential (RESP)¹⁴ charge fitting. Bonds, torsions, angles, improper torsions, and Lennard-Jones parameters were assigned using the general Amber force field¹⁵ (GAFF) using the Antechamber programs.¹⁶ QM calculations were performed using Gaussian 09.¹⁷

Docking. A previously published NMR solution structure (PDB: 6VA3) of the tau RNA in complex with a small molecule was used for docking studies after removal of the small molecule.¹⁸ Prior to grid calculations, polar hydrogen atoms and Gasteiger charges were added to RNA using AutoDock Tools 1.5.6 and MG Tools of AutoDock Vina¹⁹ and saved in pdbqt format. The 3D structures of small molecules were created with OBabel²⁰ from the corresponding SMILES input file and geometry-optimized with general AMBER force field (GAFF)¹⁶ in 5000 cycles prior to further processing for docking. Polar hydrogen atoms and Gasteiger21 charges were then added to the small molecules as described above. The Grid file was generated from ligand and receptor pdbqt files, applying prepare_gpf4.py, autogrid4 and prepare_dpf4.py script to prepare the docking parameter file. AUTODOCK-GPU²² was then used to dock the ligands against the receptor. Twenty different structures were computed with AutoDock-GPU for each molecule using the Solis Wets²³ method of search.

Calculation of accessible surface area (ASA). dr-sasa was used to calculate solvent accessible surface area.²⁴

Calculation of interactions. Interactions between RNA and compounds were extracted with fingeRNAt.²⁵

3D-RISM. 3D-RISM (three-dimensional reference interaction site model) provides the 3D map of the density distribution of the solvent around the solute.²⁶⁻²⁷ The probability density p_{γ}g_{γ}(r) of finding the interaction sites (γ) of solvent molecules positioned around the solute, in the 3D space (r) was used to represent the solvent structure. 3D-RISM calculations were performed using the AMBER16 implementation applying the Kovalenko-Hirata (KH) closure²⁸ to calculate the binding free energy along with MMGB (PB) SA.²⁹

Fluorescent Binding Affinity Measurements. Binding assays were completed as previously described.³⁰ Briefly, the RNA of interest was folded in 1× Assay Buffer-1 (AB1: 8 mM Na₂HPO₄, pH 7.0, 185 mM NaCl, and 1 mM EDTA) by heating at 95° C. for 30 s followed by slowly cooling to room temperature. Bovine serum albumin (BSA) was then added to a final concentration of 40 μg/mL to afford 1× Assay Buffer-2 (AB2: 8 mM Na₂HPO₄, pH 7.0, 185 mM NaCl, 1 mM EDTA, and 40 μg/mL BSA). The RNA solution was serial diluted with 1×AB2. Then, compound of interest was added to the RNA solutions to a final concentration of 0.5 μM. The samples were incubated at room temperature in the dark for 15 min and then transferred to a black 384-well plate (Greiner Low-Volume 784076). Fluorescence intensity was measured using a Tecan Plate Reader (Gain: 100, Integration time: 40 μs). The excitation wavelength and emission wavelengths for compound 1 are 335 nm/430 nm, respectively and for compound 2 are 330 nm/390 nm, respectively. The change in fluorescence of the small molecule was calculated by its comparison to samples lacking RNA. Binding affinity was calculated using Equation 1, as previously described:³⁰

$$I = {I}_{0} + 0.5 Δε \{ ( {[ SM ]}_{0} + {[ RNA ]}_{0} + {K}_{d} ) - {( {( {[ SM ]}_{0} + {[ RNA ]}_{0} + {K}_{d} )}^{2} - {{4 [ SM ]}_{0} [ RNA ]}_{0} )}^{0.5} \} \qquad ( Eq . 1 )$$

where I and I₀ are the measured fluorescence intensity of the compound with or without RNA, respectively, Δε is the difference between the fluorescence intensity with the infinite concentration of RNA and the fluorescence intensity without RNA, [SM]₀ and [RNA]₀ are the concentrations of the small molecule (SM) and RNA, respectively, and K_{d} is the dissociation constant.

U1 snRNA Mimic Quenching Assay. A 200 nM aliquot of dually labeled model of the DDPAC RNA (5′-FAM-GGCAGUGUGAGUACCUUCAUACGUC-BHQ-1-3′ (SEQ ID NO: 5); where FAM is 5 (6)-Carboxyfluorescein and BHQ-1 is black hole quencher) was folded in 1× Assay Buffer as described in Fluorescent Binding Affinity Measurements. Compound of interest at the indicated concentration was added, and the samples were incubated at room temperature in the dark for 15 min. A U1 snRNA mimic with sequence 5′-GUCCACUCAUGGA-3′ (SEQ ID NO: 6) was then added to the samples to a final concentration of 20 μM. The fluorescence intensity of FAM was measured as a function of time for 1 h using a Biomek FLx800 plate reader, with excitation and emission wavelengths of 485 and 525 nm, respectively.

Optical Melting Experiments. The thermal stabilities of WT, DDPAC, and DDPAC+I17T model RNAs were analyzed by optical melting in the presence or absence of compound 1 or 2 as previously described.¹⁸ The RNAs (2 μM) were heated to 95° C. in 1×PBS buffer and slowly cooled to room temperature. The samples were then cooled to 15° C. in the presence of compound 1 (10 μM; 0.1% (v/v) DMSO), 2 (1.5 μM; 0.1% (v/v) DMSO) or vehicle (0.1% (v/v) DMSO) and then heated to 85° C. at a rate of 1° C. per min. Absorbance as a function of temperature was measured at 260 nm using a Beckman Coulter DU800 UV/vis spectrophotometer. Melting curves were fitted with MeltWin 3.5.³¹

In Vitro Chemical Cross-linking and Isolation by Pull Down (Chem-CLIP) and Competitive Chem-CLIP (C-Chem-CLIP). Growth medium [Dulbecco's modified eagle medium (DMEM; corning, 10-013-CM) with 1× Glutagro (corning)] and 10% (v/v) fetal bovine serum (FBS; Sigma, 12003C) was inactivated by heating at 95° C. for 15 min and slowly cooling to room temperature. Approximately 10,000 counts of 32P 5′-end labeled tau WT, DDPAC, WT+I17T, or DDPAC+I17T RNA were folded in 10 μL growth medium by heating at 65° C. for 5 min and slowly cooling to room temperature. Chem-CLIP probe 3 or negative control probe 4 was then added into the folded RNA in a total volume of 20 μL, and the mixtures were incubated at 37° C. for 1 h. For C-Chem-CLIP, dilutions of competing compound 2 were incubated with RNA for 30 min before adding 3. The samples were then cross-linked by UV irradiation with 365 nm light in a UV Stratalinker 2400 (Stratagene) for 10 min (lids of the microcentrifuge opened).

The Chem-CLIP probe 3 or control probe 4 was then clicked to disulfide biotin azide (2 μL of 10 mM solution prepared in DMSO solution per sample; Click Chemistry Tools, catalog no. 1168-10). After addition of 20 μL of 25 mM HEPES, pH 7, a mixture of 30 μL (v/v/v=1:1:1) sodium ascorbate (250 mM in H₂O), CuSO₄ (10 mM in H₂O) and THPTA (50 mM in H₂O) was added, and the samples were incubated for 2 h at 37° C. Then, streptavidin beads (10 μL per sample; Dynabeads MyOne Streptavidin C₁ Beads, Invitrogen) were added to the mixtures for pull-down of the cross-linked RNA. The samples were shaken for 20 min at room temperature and then washed three times with 1×PBST (phosphate buffered saline containing 0.1% (v/v) Tween-20). After the incubation period and for each wash, the beads were collected by placing reaction tubes on a magnetic separation rack, and the buffer was collected and pooled. The radioactive signal from bound RNAs (on the beads) and unbound RNAs (in the wash buffer) was measured by using a Beckman Coulter LS6500 Liquid Scintillation Counter as R_{bound} and R_{unbound}. Enrichment was calculated as the percentage of radiolabeled RNA captured by beads calculated as R_{bound}/(R_{bound}+R_{unbound}).

Cell Culture. All cells were grown at 37° C. with 5% CO₂. HeLa cells were grown in growth medium [Dulbecco's modified eagle medium (DMEM; corning, 10-013-CM) with 1× Glutagro (corning)] and 10% (v/v) fetal bovine serum (FBS; Sigma, 12003C) supplemented with 1× Antibiotic/Antimycotic solution (Gibco, 15240062). Cells were discarded after 35 passages. LAN5 neuroblastoma cells were grown in RPMI 1640 medium supplemented with 1× penicillin/streptomycin solution (Corning) and 20% (v/v) FBS. The protocol for culturing primary neurons from htau mice is described in htau Mice Primary Neuron Experiments.

Cell Viability. Cells were treated with DMSO (vehicle) or compound 2 for 48 h at 37° C. After the removal of the DMSO or compound-containing growth medium, the cells were washed with 1×DPBS and incubated in growth medium containing 10% (v/v) WST-1 cell proliferation reagent (Roche) or CellTiter Fluor (Promega; per the manufacturer's recommend protocol) for 40 min. The absorbance of the solution was measured at 450 and 650 nm with a SpectraMax M5 plate reader (Molecular Devices) for cells treated with WST-1. The background signal at 650 nm was subtracted from the WST-1 signal at 450 nm for analysis. For cells treated with CellTiter Fluor, the fluorescence was measured with 390 nm excitation and 505 nm emission wavelengths. Cells treated with a compound were compared to vehicle treated cells to determine the effect of the compound on cell viability.

Luciferase-reporter Assay to Study Exon 10 Alternative Splicing. The exon 10 mini-genes, both WT and DDPAC and mutant thereof, fused to firefly luciferase, have been previously described.¹⁸ HeLa cells were seeded in 60 mm diameter dishes and incubated for 12-16 h to 70% confluency. They were then transfected with WT, WT+I17T, DDPAC, or DDPAC+I17T luciferase reporter minigenes using JetPrime (Polyplus Transfection) per the manufacturer's recommended protocol and incubated for 4 h. After transfection, the cells were collected by trypsinization and re-seeded in 96-well plates at 0.2×10⁵ cells per well, followed by treatment of compounds after 4 h. Compound stocks prepared in DMSO were diluted in growth medium and added to cells with final DMSO concentration at 0.1% (v/v). After a 48 h treatment period, the cells were washed with 1× DPBS. Each well was added with a mixture of 10 μL WST-1 reagent and 90 μL of growth medium. After incubation at 37° C. for 45 min, absorbance was measured at 450 and 690 nm. A₆₉₀ as background was subtracted from A₄₅₀ to obtain A₄₅₀′. After removal of WST-1/DMEM from wells, the cells were washed again with 1×DPBS and treated with 100 μL of One-Glo EX reagent (Promega, E8110) per well. The plate was incubated at room temperature for 20 min and the luminescence was measured with a Biomek FLx800 plate reader (1000 ms integration time). The resulted luminescence was normalized to A₄₅₀′.

RNA Isolation and Real-Time Quantitative PCR (RT-qPCR). HeLa cells were treated as described in the Luciferase Reporter assay to Study Exon 10 Alternative Splicing. LAN5 cells were seeded in 12-well plates at 0.2×10⁶ cells per well, treated with compound or ASO, and incubated for 48 h. Compound stocks prepared in DMSO and ASO stocks prepared in water were diluted in growth medium and added to cells. Total RNA was extracted from cells and analyzed via RT-qPCR to determine levels of 3R and 4R tau mRNA. In brief, the cells were washed with 1×DPBS and then lysed in the well using 300 μL of Lysis Buffer provided in a Quick-RNA MiniPrep kit (Zymo Research), which was used to extract total RNA per the manufacturer's protocol including the on-column DNase I digestion. The concentration of total RNA was measured by NanoDrop 2000 (Thermo Fisher Scientific). Only samples with OD₂₆₀/OD₂₈₀>1.8 were carried forward for analysis. Reverse transcription (RT) was carried out on 200 ng of total RNA using a qScript cDNA synthesis kit (QuantaBio) in a total volume of 10 μL according to the manufacturer's protocol. An Applied Biosystems QS5 384-well PCR system was used to complete qPCR reactions with Power SYBR Green Master Mix (Life Technologies), 600 nM each primer (Table S3), 20 ng of cDNA (1 μL of the RT reaction), in a total volume of 33 μL. Technical triplicates (10 μL each) were aliquoted into 384-well qPCR plates (Applied biosystems, 4483285). Subsequent qPCR analysis was performed using an Applied Biosystems QS5 384-well PCR system (software v.1.3.0). 4R/3R ratio were analyzed using ΔΔCₜ method using Equation 2:

$$4 R / 3 R ratio = {2}^{- ( Δ {C}_{t} 4 R - Δ {C}_{t} 3 R )} \qquad ( Eq . 2 )$$

where ΔCₜ 4R or ΔCₜ 3R is the difference between the Ct values for the 4R or 3R MAPT mRNA and a housekeeping gene (18S rRNA) in total RNA from cells.

Measuring Cellular Occupancy by Chem-CLIP and C-Chem-CLIP. LAN5 cells were grown to ~80% confluency in 100 mm diameter dishes. HeLa cells were transfected as described in the Luciferase Reporter Assay to Study Exon 10 Alternative Splicing except they were re-plated in 100 mm diameter dishes and grown to ~80% confluency (~1 day). The cells were then treated with 1.5 μM of 3 or 4 for 12 h. For C-Chem-CLIP, dilutions of competing compounds 1 or 2 were pre-incubated with cells for 12 h prior to adding 3. After addition of 3, the cells were incubated for an additional 12 h. After the treatment period, whether for Chem-CLIP or C-Chem-CLIP studies, the cells were washed with 10 mL of 1×DPBS. Ice-cold 1×DPBS (10 mL) was then added to the plate followed by irradiation with 365 nm light in a UV Stratalinker 2400 (Stratagene). Cells were scraped with a cell scraper, transferred to a 15 mL conical vial, and collected by centrifugation. Total RNA was extracted using TRIzol LS Reagent (Invitrogen) per the the manufacturer's protocol.

Approximately 20 μg of total RNA was then subjected to the click reaction with biotin azide and pull-down with magnetic streptavidin beads as described in In Vitro Chemical Cross-linking and Isolation by Pull Down (Chem-CLIP) and Competitive Chem-CLIP (C-Chem-CLIP). After the washing steps, the cross-linked RNA captured by the beads was cleaved using a 1:1 mixture of TCEP (200 mM) and K₂CO₃ (600 mM) by incubating at 37° C. for 30 min with shaking. The reaction was quenched with 1 volume of Iodoacetamide (400 mM) by shaking at room temperature for 30 min. The isolated RNA was cleaned up by using RNA Clean XP beads (Beckman Coulter) according to manufacturer's protocol.

RT-qPCR was performed on RNA samples before and after pull-down as described in Real-Time Quantitative PCR to enable calculation of fold enrichment in the pulled down samples. Relative fold enrichment of tau pre-mRNA was measured using Equation 3:

$$Relative Fold Enrichment = {2}^{- ( Δ {C}_{t} before pull - down - Δ {C}_{t} after pull - down )} \qquad ( Eq . 3 )$$

where “ΔCₜ before pull-down” is the difference between the Cₜ values for the MAPT mRNA and 18S rRNA in total RNA from cells and “ΔCₜ after pull-down” is the difference between the Cₜ values for the MAPT pre-mRNA and 18S rRNA in RNA in the pulled down fractions.

Chem-CLIP-Map-Seq. Chem-CLIP-Map-Seq was performed as previously described.³² Briefly, LAN5 cells were grown to ~80% confluency in 100 mm diameter dishes and treated with 1.5 μM of 3 for 12 h. Total RNA was extracted and pulled down as described in Measuring Cellular Occupancy by Chem-CLIP and C-Chem-CLIP. RT was carried out using 1 μg of RNA pulled down by 3, 2 μmol of a MAPT gene-specific primer (5′-CAGACGTGTGCTCTTCCGATCTGACACTCCAGTCCCACAGT (SEQ ID NO: 7)), and SuperScript III (Invitrogen, 18080400) in a total volume of 10 μL per the manufacturer's protocol.

After digestion with RNase A and RNase H (part of the Superscript III protocol), the cDNA from the RT reaction was cleaned up using RNA Clean XP beads (Beckman Coulter) according to the manufacturer's protocol. The purified cDNA was ligated to a 3′ ssDNA adaptor (5′-Phosphate-NNNAGATCGGAAGAGCG-TCGTGTAG-3C spacer (SEQ ID NO: 8)) by incubating 2 μL 10× T4 RNA Ligase Buffer, 1 μL of 1 mM ATP, 10 μL 50% (w/v) PEG 8000, 5 μL cDNA, 1 μL of 20 μM ssDNA adaptor, and 1 μL of T4 RNA Ligase [New England BioLabs, Inc. (NEB)]. The ligated cDNA was purified again with RNAClean XP beads as described above.

PCR amplification was performed with the purified ligated cDNA by using Phusion polymerase (NEB) with cycling conditions of 98° C. for 30 s, 65° C. for 20 s, and 72° C. for 60 s and forward (5′-CAGACGTGTGCTCTTCCGATC (SEQ ID NO: 9)) and reverse (5′-CTACACGACGCTCTTCCGATCT (SEQ ID NO: 10)) primers for 25 cycles. The PCR products were separated on a denaturing 15% (w/v) polyacrylamide gel, and the band (~1.5k bp) was excised from the gel and purified by ethanol precipitation. A second round PCR was performed to amplify the purified DNA. About 100 ng of purified DNA was ligated into a vector using NEB's PCR Cloning Kit (E1202S) per the manufacturer's protocol. Ampicillin-resistant colonies were selected and subjected to Sanger sequencing by Eton.

Western Blotting. LAN5 cells were seeded in 6-well plates at 0.6×10⁶ cells per well, treated with compound and incubated for 48 h. After removing the growth medium, the cells were washed with 1×DPBS and harvested by trypsinization. Total protein was extracted with Mammalian Protein Extraction Reagent (M-PER, Thermo Scientific) per the manufacturer's protocol. Protein concentrations were determined using a Pierce Micro BCA Protein Assay Kit per the manufacturer's recommended protocol. Approximately 20 μg of total protein from each biological sample was separated on a sodium dodecyl sulfate (SDS)-polyacrylamide gel (5% (w/v) polyacrylamide stacking layer; 12% (w/v) polyacrylamide separating layer), followed by transferring to a polyvinylidene fluoride (PVDF; 0.45 μm) membrane. After blocking the membrane in 1×TBST with 5% (w/v) nonfat milk for 1 h, the membrane was incubated with 1×TBST containing 5% (w/v) milk containing a primary antibody as follows: 4R tau antibody (MilliporeSigma, 05-804; 1:2000 dilution) or 3R tau antibody (MilliporeSigma, 05-803; 1:5000 dilution) and incubated at 4° C. overnight. The membrane was washed three times with 1×TBST for 10 min each and then incubated with a horseradish peroxidase (HRP)-conjugated secondary antibody (Cell Signaling Technology, 7076; 1:5000 dilution) in 1×TBST containing 5% (w/v) milk for about 1 h at room temperature. After three more washes with 1×TBST for 15 min each, 4R, 3R tau protein, or B-actin were detected using SuperSignal West Pico Chemiluminescent Substrate (Pierce Biotechnology).

After imaging, the membrane was stripped with 1× Stripping Buffer (200 mM glycine with 0.1% SDS, pH 2.2) for 0.5 h at room temperature, and β-actin levels were detected as described above, except using a β-actin primary antibody (Cell Signaling Technology, 4970S; 1:5000 dilution). ImageJ software was used for the quantification of the protein bands.

RNA-Seq. LAN5 cells in 12-well plate (~60% confluency) were treated with 2 (1.5 μM), 0.1% (v/v) DMSO (vehicle), 4R-to-3R tau ASO (0.5 μM), or scrambled ASO (0.5 μM) for 48 h. Total RNA was extracted using an RNeasy Mini Kit (Qiagen) per the manufacturer's protocol. Next, rRNA was depleted using NEBNext rRNA depletion modules (E6310L, New England Biosciences) and prepared with NEBNext Ultra II Dir RNA Lib Prep Kit (E7760S, New England Biosciences) according to the manufacturer's protocol. The prepared libraries were processed with NextSeq 500/550 High Output Kit v2.5 (Illumina, 20024907) per the manufacturer's protocol and then sequenced on a NextSeq 500. After sequencing, the BCL files were converted to FASTQ files using basemount/0.12.8.1516 and bcl2fastq/2.17.1.14.³³ The sequencing depth was over 82 million for each sample.

FastQC³⁴ and RSeQC³⁵ were used to determine the quality of reads and any overrepresentation of sequences. HISAT2³⁶ was used to map the reads to the reference human genome (hg19), and the output SAM files were converted to BAM files and sorted by index. Kallisto³⁷ and NCBI reference sequences (RefSeq)³⁸ were used for gene quantification to calculate transcripts per million (TPM). Differentially expressed genes were detected with Sleuth³⁹ and determined by a multiple test corrected p-value<0.05. Splicing event PSI values were quantitated using MISO.⁴⁰

Isolation and Treatment of Primary Neurons from Humanized tau (hTau) Transgenic Mice. All animal studies were completed as approved by the Scripps Florida Institutional Animal Care and Use Committee. All the required Dissection Medium, Plating Medium, Feeding Medium, and miscellaneous reagents were prepared as previously described.¹⁸ Culture plates (Corning CellBIND 24-well Surface Microplates, 3337) were pre-coated with 0.1 mg/mL Poly-D-Lysine (PDL), which was dissolved in 10 mM TRIS buffer (pH=7.4), for 2 h at 37° C. The amounts of media required for isolation and culture of primary neurons are as follows: 1 mL per pup of Dissection Medium, placed on ice; another 1 mL per pup of Dissection Medium supplemented with 1% (v/v) Papain pre-warmed at 37° C.; 5 mL per pup of Plating Medium for the dissociation step and 25 mL of Feeding Medium per plate pre-warmed at 37° C.

P0 htau mouse pups were obtained from the breeding of [B6.Cg-Mapttm1 (EGFP) Klt Tg(MAPT)8cPdav/J] mice which acquired from Jackson Labs and genotyped by PCR amplification of DNA extracted from mouse tails. For one 24-well plate, the cortex of three P0 htau mouse pups were removed and each placed in the 1.6 mL eppendorf tube containing 1 mL of ice-cold Dissection Medium for dissection and removal of meninges. After removal of the meninges as previously described,⁴¹ the cold Dissection Medium was aspirated and 1 mL of Dissection Medium containing Papain solution pre-warmed to 37° C. was added into each tube. The samples were incubated at 37° C. for 20 min, and then 1 mL of Plating Medium was added to each tube to quench Papain activity. During the Papain quenching, PDL-coated plate was washed with 1×DPBS, and 12 mL of Feeding Medium was added (0.5 mL per well), and the plate was incubated at 37° C.

The Dissection and Plating Medium in each 1.6 mL eppendorf tube was aspirated, and the remaining tissue was washed three times with 1 mL of pre-warmed Plating Medium. After the washing steps, 2 mL of freshly prepared Plating Medium was added, and the tissue was dissociated by gently triturating through a 5 mL serological pipette three times and then a P-1000 pipet three times. One mL of the supernatant was transferred to a new 15 mL tube, to which was added 1 mL of freshly prepared Plating Medium. Trituration was repeated three to six times using 1 mL of Plating Medium and a P-1000 pipet until the tissue was homogenized and all chunks of tissue were dissociated; 1 mL supernatant was transferred each time from each 15 mL tube and collected in a new 15 ml conical vial separately. The cell mixture was diluted to 10 mL with Plating Medium and strained through a 40 μm cell strainer into a 50 mL tube. The cells were centrifuged at 1900 rpm for 4 min, and the three pellets were combined and resuspended in 1 mL freshly prepared Plating Medium for cell counting. The cells were diluted to 0.6×10⁶/mL with Feeding Medium supplemented with 2% (v/v) B-27 (Gibco, 17504044). Diluted 12 mL Feeding Medium was added in the pre-warmed PDL-coated plates (0.5 mL per well). Half of the Feeding Medium supplemented with 2% (v/v) B-27 was replaced every 3-4 days. After 15 days, the time period required for detectable levels of 3R and 4R tau, neurons were treated with 1, 2, tau ASO, and scrambled ASO for 48 h at 37° C. Total RNA was extracted and RT-qPCR was performed as described in RNA Isolation and Real-Time Quantitative PCR (RT-qPCR).

Quantitation of 2 in Mouse Plasma and Brain. Male C₅₇BL/6J mice (n=3 per time point; 5-7 weeks) were orally administered with 2 (100 mg/kg) in a formulation of DMSO/Tween-80/H₂O (5/5/90). After 2, 12, 24, and 48 h, mice were euthanized, and blood and brain were collected. Brains were immediately frozen. Blood was centrifuged to generate plasma and immediately frozen. On the day of analysis, brains were homogenized, mixed with a 5-times volume of acetonitrile, and filtered. The plasma was also filtered. Drug levels were determined by mass spectrometry using an ABSciex 5500 mass spectrometer with multiple reaction monitoring. Compound 2 was detected using the mass transition 435->236.

Plasma and brain binding. Plasma protein binding was determined by equilibrium dialysis using a Thermo Scientific™ RED Device per the manufacturer's protocol. Each sample was prepared by addition of 2 into 100 μL plasma with final concentration of 5 μM (0.1% (v/v) DMSO). 1×PBS was used as dialysis buffer and was added into the buffer chamber. The plate was incubated with shaking at 37° C. for 6 h. The concentration of 2 in the plasma and buffer compartments was determined as described in Quantitation of 2 in Mouse Plasma and Brain. The free fraction was calculated as [buffer]/[plasma].

hTau Mice Studies. htau mice and WT mice (C₅₇BL/6J) were purchased from Jackson Laboratories. htau mice were maintained in-house through breeding between htau+/− mice. Genotype was confirmed by PCR amplification as previously described.¹⁸ Only homozygous mice were used in in vivo studies. Mice were randomly assigned to a treatment group, which were age- and gender-matched (n=X). In these studies, mice were >9 months of age since cognitive and physiological impairments in htau mice was age-dependent and were not presented in young mice (<4 months) with early-stage tau pathology.⁴² Mice were orally administered vehicle (5/5/90 DMSO/Tween-80/H₂O) or 100 mg/kg of 2 in the same formulation every other day (q.o.d.). During the treatment period, nesting activity was assessed, as describe in Nesting Activity below. After 20 days, the mice were euthanized (in accordance with guidelines provided by the American Veterinarian Medical Association), and the brain was harvested for analysis. One hemisphere of the brain was frozen at −80° C. for RT-qPCR analyses while the other was used for histological studies.

Nesting Activity. From Day 1 to Day 20, mice were individually housed and nesting material from the previous day was removed. An intact 3.0 g nestlet was placed within each cage. Around 15 h later, the resulting nest was photographed for qualitative scoring, and the untorn nestlet was weighed if present. All nest images and weights were analyzed by a blinded individual and based on published criteria.⁴³ In brief, nesting is scored on a rating scale of 1-5 where if the nestlet is over 90% intact, it is given a nestlet score is 1 (Poor; FIG. 4C). When the nestlet is partially torn but >50% remains, the nestlet score is 2; a score of 3 is assigned when 50-90% of the nestlet is torn but spread around the cage and no identifiable nest site is found (Medium; FIG. 4C). When more than 90% of the nestlet is torn and the nest is identifiable but flat, the nestlet score is 4. When a near perfect nest is built and the wall is higher than the mouse's body, it is assigned a nestlet score of 5 (Good; FIG. 4C).

Brain Tissue Histology. Left brain hemispheres of htau mice were harvested for total RNA. Right brain hemispheres (unfrozen) of htau mice were stored in 10% neutral buffered formalin (VWR) for 48 h. Tissue processing, embedding, and sectioning were performed and generated by the Scripps Florida Histology Core. Briefly, tissue was embedded in paraffin using a Sakura Tissue-Tek VIP5 paraffin processor, sectioned at 4 μm, and then mounted on positively charged slides for further immunostaining. The slides were stained on a Leica BondMax Immunostaining platform with the following primary antibodies: AT8 (MN1020, Invitrogen; 1:100 dilution) and NeuN (MAB377B, Chemicon; 1:500 dilution). After the slides were washed three times with 1×DPBS, a DAB Substrate Kit (Vector Laboratories, Inc.) was used to detect the primary antibodies per the manufacturer's protocol. After staining was complete, the slides were dehydrated, cover-slipped with Cytoseal 60 (Thermo Scientific), and imaged with a Leica DMI3000 B upright fluorescent microscope using 20× or 60× objective. Quantification was performed in a blinded fashion using ImageJ in which positivity was identified as aggregates (purple staining of AT8 or brown staining of NeuN) with diameter over 20 μm and staining signal over 185 that were kept constant across all treatment groups.

Statistical Analysis. All data are reported as the mean±standard deviation (SD). Data were plotted and analyzed using commercially available software (Perseus, GraphPad Prism, and ImageJ). Statistical significance between experimental groups was analyzed either by two-tailed Student's t-test or one-way ANOVA followed by Bonferroni's multiple-comparison test. In all cases, p-values of less than 0.05 were considered to be statistically significant.

TABLE 1
Calculation of CNS-MPO and binding energy
(ΔG°₃₇) of compounds used in the present disclosure.

| | CNS-MPO | Binding energy |
|---|---|---|
| Compound | value^{a} | (kcal/mol)^{b} |
| 1 | 5.43 | −8.91 |
| 2 | 4.30 | −9.19 |
| M1 | 5.0 | −6.39 |
| S1 | 3.64 | −11.42 |
| S2 | 2.74 | −12.97 |
| S3 | 2.96 | −13.57 |
| S4 | 2.71 | −12.76 |
| S5 | 2.14 | −12.29 |
| S6 | 2.12 | −12.35 |
| S7 | 4.46 | −9.15 |
| S8 | 4.31 | −9.19 |

^{a}CNS-MPO sores were calculated using the implementation of Chemaxon.
^{b}lowest free energy docking pose extracted from Autodock.

TABLE 2
Free energy calculation of the first 100 clusters of 2. Rism
was used to calculate the free energy of the bound states
of the first 100 clusters with a minimum of 100 structures.

| Cluster # | ΔG_{RISM} | Cluster # | ΔG_{RISM} | Cluster # | ΔG_{RISM} |
|---|---|---|---|---|---|
| 16 | −20.0385 | 36 | −15.5342 | 73 | −12.6870 |
| 3 | −19.1309 | 77 | −15.5275 | 80 | −12.2771 |
| 41 | −18.9802 | 78 | −15.5185 | 90 | −12.2113 |
| 21 | −18.9724 | 29 | −15.4620 | 52 | −12.0081 |
| 6 | −18.7596 | 14 | −15.4530 | 54 | −11.9202 |
| 48 | −18.3165 | 51 | −15.3080 | 33 | −11.7872 |
| 10 | −18.1301 | 15 | −15.0693 | 99 | −11.7288 |
| 4 | −18.0512 | 0 | −15.0174 | 62 | −11.3820 |
| 100 | −17.9325 | 93 | −14.9522 | 55 | −11.1613 |
| 32 | −17.7566 | 75 | −14.9455 | 94 | −11.0715 |
| 8 | −17.5080 | 37 | −14.7530 | 83 | −10.9736 |
| 1 | −17.4271 | 40 | −14.7144 | 35 | −10.9243 |
| 17 | −17.3956 | 88 | −14.5433 | 64 | −10.1825 |
| 26 | −17.3750 | 13 | −14.3551 | 72 | −9.8956 |
| 87 | −17.3603 | 59 | −14.3140 | 18 | −9.5742 |
| 43 | −17.3524 | 31 | −14.3108 | 81 | −9.5690 |
| 95 | −17.2698 | 71 | −14.3096 | 67 | −9.5645 |
| 25 | −17.2129 | 49 | −14.0587 | 65 | −9.5006 |
| 38 | −17.1764 | 79 | −13.9413 | 39 | −9.3982 |
| 5 | −17.0453 | 28 | −13.9382 | 84 | −9.3144 |
| 7 | −16.8494 | 58 | −13.8899 | 76 | −9.2738 |
| 23 | −16.8208 | 46 | −13.8798 | 69 | −9.2691 |
| 34 | −16.4681 | 86 | −13.7486 | 45 | −9.2490 |
| 56 | −16.4535 | 66 | −13.5303 | 24 | −9.2360 |
| 9 | −16.3005 | 27 | −13.1813 | 96 | −8.8909 |
| 2 | −16.2124 | 19 | −13.0964 | 57 | −8.8818 |
| 11 | −16.2034 | 44 | −13.0564 | 74 | −8.7341 |
| 82 | −16.1283 | 47 | −13.0385 | 20 | −8.6791 |
| 50 | −15.8621 | 70 | −12.8535 | 12 | −8.6702 |
| 98 | −15.6051 | 60 | −12.8524 | 30 | −8.6700 |
| 36 | −15.5342 | 42 | −12.8402 | 68 | −8.6379 |
| 77 | −15.5275 | | | 22 | −8.5919 |
| 78 | −15.5185 | | | 61 | −8.5628 |
| 29 | −15.4620 | | | 97 | −8.5057 |
| 14 | −15.4530 | | | 53 | −8.4107 |
| 51 | −15.3080 | | | 91 | −8.3540 |
| 15 | −15.0693 | | | 85 | −8.1336 |
| 0 | −15.0174 | | | 63 | −8.0024 |
| 93 | −14.9522 | | | 92 | −7.3036 |
| 75 | −14.9455 | | | 89 | −4.8459 |
| 37 | −14.7530 | | | | |
| 40 | −14.7144 | | | | |
| 88 | −14.5433 | | | | |
| 13 | −14.3551 | | | | |
| 59 | −14.3140 | | | | |
| 31 | −14.3108 | | | | |
| 71 | −14.3096 | | | | |
| 49 | −14.0587 | | | | |
| 79 | −13.9413 | | | | |
| 28 | −13.9382 | | | | |
| 58 | −13.8899 | | | | |
| 46 | −13.8798 | | | | |
| 86 | −13.7486 | | | | |
| 66 | −13.5303 | | | | |
| 27 | −13.1813 | | | | |
| 19 | −13.0964 | | | | |
| 44 | −13.0564 | | | | |
| 47 | −13.0385 | | | | |
| 70 | −12.8535 | | | | |
| 60 | −12.8524 | | | | |
| 42 | −12.8402 | | | | |

TABLE 3
Primer sequences used in the present disclosure.
F denotes forward primer and R denotes reverse primer.

| | | SEQ. | Sequence |
|---|---|---|---|
| Purpose | F/R | ID No. | |
| 4R Tau mRNA in | F | 11 | 5′- |
| transfected HeLa cells | | | GAGGCGGGAAGGTGCAGATAATTAATAAGA- |
| | | | 3′ |
| 3R Tau mRNA in | F | 12 | 5′-CAGCCGGGAGGCGGGAAGGTGCAAATAG- |
| transfected HeLa cells | | | 3′ |
| 4R and 3R Tau mRNA in | R | 13 | 5′-GCCTTATGCAGTTGCTCTCC-3′ |
| transfected HeLa cells | | | |
| 4R Tau in LAN5 cells | F | 14 | 5′-GAGGCGGGAAGGTGCAGATAATTAATAA-3′ |
| 4R Tau in LAN5 cells | R | 15 | 5′-CTGGTTTATGATGGATGTTGCC-3′ |
| 3R Tau in LAN5 cells and | F | 16 | 5′-GAAGAATGTCAAGTCCAAGATCGG-3′ |
| mouse | | | |
| 3R Tau in LAN5 cells and | R | 17 | 5′-GACTATTTGCACCTTCCCGC-3′ |
| mouse | | | |
| Tau pre-mRNA in Chem- | F | 18 | 5′-GGAAGTGGTGTGAGTGCGTACAC-3′ |
| CLIP experiments | | | |
| Tau pre-mRNA in Chem- | R | 19 | 5′-CACCTTCAGCCCAACTTCCAATG-3′ |
| CLIP experiments | | | |
| Total Tau in mouse | F | 20 | 5′-AGAAGCAGGCATTGGAGAC |
| Total Tau in mouse | R | 21 | 5′-TCTTCGTTTTACCATCAGCC |
| GAPDH in mouse | F | 22 | 5′-TGCCCCCATGTTGTGATG |
| GAPDH in mouse | R | 23 | 5′-TGTGGTCATGAGCCCTTCC |
| 4R Tau in mouse | F | 24 | 5′-CACTGAGAACCTGAAGCACC-3′ |
| 4R Tau in mouse | R | 25 | 5′-GGACGTTGCTAAGATCCAGCT-3′ |
| GAPDH | F | 26 | 5′-GGCAAATTCAACGGCACAGT-3′ |
| GAPDH | R | 27 | 5′-GGGTCTCGCTCCTGGAAGAT-3′ |
| β-actin | F | 28 | 5′-CATGTACGTTGCTATCCAGGC-3′ |
| β-actin | R | 29 | 5′-CTCCTTAATGTCACGCACGAT-3′ |
| 18S rRNA | F | 30 | 5′-GTAACCCGTTGAACCCCATT-3′ |
| 18S rRNA | R | 31 | 5′-TCCAATCGGTAGTAGCG-3′ |
| hTau mice Genotyping | F | 32 | 5′-ACTTTGAACCAGGATGGCTGAGCCC-3′ |
| hTau mice Genotyping | R | 33 | 5′-CTGTGCATGGCTGTCCACTAA CCT T-3′ |

### Example 3: Synthetic Schemes, Methods, and Characterizations

Abbreviations. AcOH, acetic acid; Boc, tert-butyloxycarbonyl; Cbz, benzyloxycarbonyl; DCM, dichloromethane; DIEA, diisopropylethylamine; DMF, dimethylformamide; HATU, hexafluorophosphate azabenzotriazole tetramethyl uronium; HPLC, high performance liquid chromatography; MeOH, methanol; NIS, N-Iodosuccinimide; TFA, trifluoroacetic acid; TLC, thin layer chromatography.

General methods. Reagents and solvents were purchased from commercial sources and used without further purification. Reactions were monitored by thin layer chromatography (TLC, Agela Technologies) or by LC-MS. Bands on TLC were visualized under UV light (254 nm). Compound 2 was purified by Isolera One Flash Chromatography System (Biotage) using pre-packed C18 column (spherical 20-35 μm, Agela Technologies). Preparative HPLC purification for 3 was performed by HPLC (Waters 2489 and 1525) using a SunFire® Prep C18 OBD™ 5 μm column (19×150 mm) with a 5 mL/min flow. NMR spectra were collected on a 400 UltraShield™ (Bruker) (400 MHz for 1H and 100 MHz for ¹³C) or Ascend™ 600 (Bruker) (600 MHz for 1H and 150 MHz for ¹³C). Chemical shifts are reported in ppm relative to tetramethylsilane (TMS) for 1H and residual solvent for ¹³C as internal standards. Coupling constant (J values) are expressed in Hz. High resolution mass spectra of all compounds were recorded on a 4800 Plus MALDI TOF/TOF Analyzer (Applied Biosystems) with a-cyano-4-hydroxycinnamic acid matrix and TOF/TOF Calibration Mixture (AB Sciex Pte. Ltd) or on an Agilent 1260 Infinity LC system coupled to an Agilent 6230 TOF (HR-ESI) with a Poroshell 120 EC-C18 column (Agilent, 50 mm×4.6 mm, 2.7 μm). The synthesis and characterization of 1 and 4 has been previously reported.¹⁸

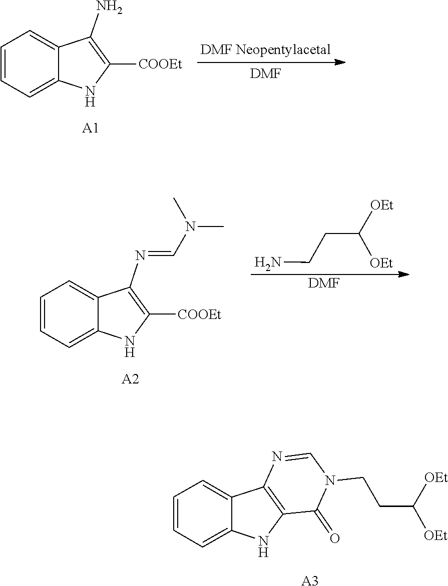

3-(3,3-diethoxypropyl)-3,5-dihydro-4H-pyrimido[5,4-b]indol-4-one (A3): A mixture of ethyl 3-amino-1H-indole-2-carboxylate (A1) (5.00 g, 21.3 mmol) and DMF Neopentylacetal (20.8 mL, 74.7 mmol) in DMF (75 mL) was stirred at 140° C. for 10 h. The reaction was concentrated in vacuo and azeotroped with toluene (3×5 mL) to give A2. This intermediate was added into 3,3-diethoxypropan-1-amine (20 mL), and the mixture was stirred at 100° C. for 3 h. After cooling to room temperature, the reaction mixture was concentrated in vacuo. To this mixture, H₂O (20 mL) was added, and the mixture was filtered to give 3-(3,3-diethoxypropyl)-3,5-dihydro-4H-pyrimido[5,4-b]indol-4-one (A3) (6.00 g, 89% (2 steps)). The material was used in the next reaction without further purification. ¹H NMR (400 MHz, MeOD) δ 8.26 (s, 1H), 8.10-8.07 (m, 1H), 7.60-7.55 (m, 1H), 7.52-7.47 (m, 1H), 7.29-7.24 (m, 1H), 4.64 (t, J=5.2 Hz, 1H), 4.27 (t, J=6.8 Hz, 2H), 3.68-3.60 (m, 2H), 3.55-3.44 (m, 2H), 2.19-2.10 (m, 2H), 1.11 (t, J=7.0 Hz, 6H) ¹³C NMR (150 MHz, DMSO-d₆) δ 154.6, 144.9, 139.3, 138.0, 127.5, 122.3, 121.5, 120.7, 120.7, 113.3, 100.9, 61.2 (2C), 42.8, 33.2, 15.7 (2C); HR-MS (ESI): Calcd for C₁₇H₂₀N₃O₃⁻ [M−H]⁻; 314.1510; found, 314.1523.

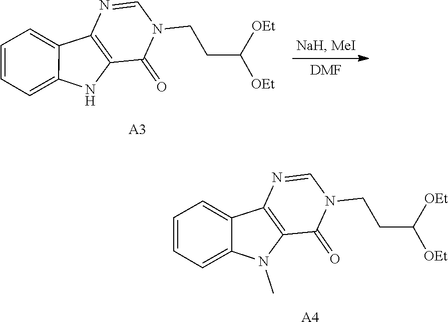

3-(3,3-diethoxypropyl)-5-methyl-3,5-dihydro-4H-pyrimido[5,4-b]indol-4-one (A4): To a solution of A3 (6.00 g, 19.0 mmol) in DMF (60.0 mL), NaH (1142 mg, 28.5 mmol) was added at 0° C. and the mixture was stirred at 0° C. for 10 min. Mel (3.53 mL, 38.1 mmol) was then added at 0° C. The reaction mixture was stirred at 0° C. for 25 min, then H₂O (100 mL) was added, and the mixture was extracted with MeOH/DCM (1/9, 3×100 mL) and concentrated in vacuo. The reaction mixture was purified by column chromatography (Agela Technologies, Silica, 20 g, 10%-30% ethylacetate in hexane) to afford 3-(3,3-diethoxypropyl)-5-methyl-3,5-dihydro-4H-pyrimido[5,4-b]indol-4-one (A4) (4.98 g, 79%). ¹H NMR (400 MHz, CDCl₃) δ 8.15 (dd, J=8.0, 0.90 Hz, 1H), 8.01 (s, 1H), 7.58-7.52 (m, 1H), 7.46 (d, J=8.4 Hz, 1H), 7.34-7.29 (m, 1H), 4.57 (t, J=5.4 Hz, 1H), 4.27 (s, 3H), 4.19 (t, J=6.9 Hz, 2H), 3.72-3.62 (m, 2H), 3.55-3.46 (m, 2H), 2.20-2.14 (m, 2H), 1.20 (t, J=7.1 Hz, 6H) ¹³C NMR (150 MHz, MeOD) δ 155.2, 144.4, 140.5, 137.3, 127.5, 121.3, 120.5, 120.2, 120.1, 109.9, 101.1, 61.5 (2C), 42.8, 32.5, 30.2, 14.1 (2C); HR-MS (ESI): Calcd for C₁₈H₂₄N₃O₃⁺ [M+H]⁺; 330.1812; found, 330.1827.

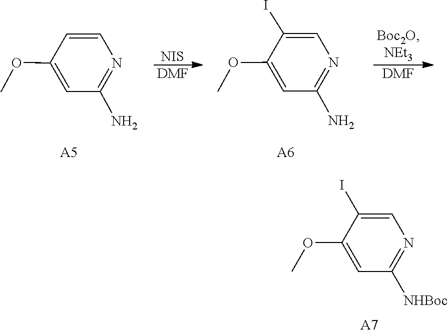

tert-butyl (5-iodo-4-methoxypyridin-2-yl)carbamate (A7): To a solution of 4-methoxypyridin-2-amine (A5) (25.0 g, 201 mmol) in DMF (400 mL), NIS (54.4 g, 225 mmol) was added at 0° C. The mixture was stirred at room temperature overnight. The reaction was then concentrated in vacuo. Then, DCM/saturated NaHCO₃(v/v, 400 mL) was added, and the resultant solids were separated by filtration. The resultant DCM/saturated NaHCO₃ mixture was extracted with DCM (400 mL×3) and concentrated in vacuo. This material was washed with H₂O (200 mL) to give 5-iodo-4-methoxypyridin-2-amine (A6) (33.0 g). To a suspension of A6 (15.0 g, 60.0 mmol) and NEt₃ (13.4 mL, 66.0 mmol) in DCM (300 mL), Boc₂O (14.4 g, 66.0 mmol) in DCM (150 mL) was added at 0° C. The mixture was stirred at room temperature overnight. Additional reagents (NEt₃ (2.43 mL, 12.0 mmol) and Boc₂O (2.62 g, 12.0 mmol)) were then added and the mixture was stirred at room temperature for 5 h. Then, 400 mL of silica gel was added, and the mixture was concentrated in vacuo. This was purified by column chromatography (Silica gel 1.0 L, 10% ethyl acetate in DCM), and the resultant product was washed with hexane (8× v/w) and filtered to give tert-butyl (5-iodo-4-methoxypyridin-2-yl)carbamate (A7) (8.64 g, 27% (2 steps)). ¹H NMR (400 MHz, CDCl₃) δ 9.51 (s, 1H), 8.46 (s, 1H), 7.67 (s, 1H), 3.98 (s, 3H), 1.57 (s, 9H) ¹³C NMR (150 MHz, DMSO-d₆) δ 165.2, 154.9, 154.8, 153.1, 96.3, 80.4, 77.3, 56.6, 28.5 (3C); HR-MS (ESI): Calcd for C₁₁H16IN₂O₃⁺ [M+H]⁺; 351.0200; found, 351.0217.

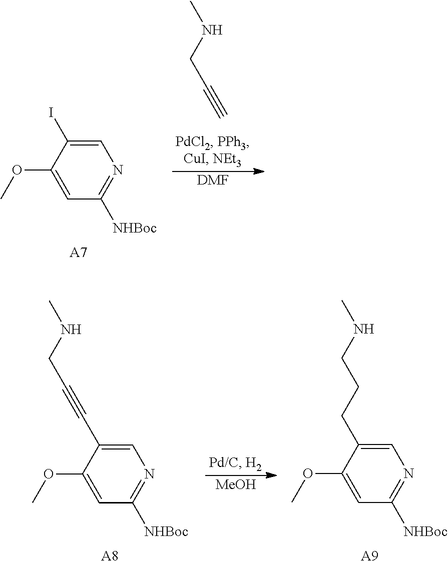

tert-butyl (4-methoxy-5-(3-(methylamino) propyl)pyridin-2-yl)carbamate (A9): A mixture of A7 (3.00 g, 8.57 mmol), PdCl₂ (103 mg, 0.343 mmol), PPh₃ (180 mg, 0.685 mmol), CuI (131 mg, 0.685 mmol), NEt₃ (8.44 mL, 60.6 mmol) and N-methylprop-2-yn-1-amine (1.78 g, 25.7 mmol) in DMF (45 mL) was stirred at 50° C. for 1 h. After cooling to room temperature, saturated NaHCO₃ (100 mL) was added, and the mixture was extracted with ethyl acetate (3×100 mL), and concentrated in vacuo. The reaction mixture was purified by column chromatography (Biotage SNAP cartridge, KP-NH, 28 g, 2%-18% MeOH in DCM) to afford A8. This was washed with hexane/Et₂O (1/1, x10v/w) to give A8 (2.13 g). A mixture of A8 (2.08 g, 7.12 mmol) and Pd/C (10%, 2.08 g) in MeOH (104 mL) was stirred at room temperature under H₂ (1 atm) for 1 h. The reaction was filtered through Celite and concentrated in vacuo. The resultant solid was washed with hexane/Et₂O (1/1, x10v/w) and filtered to give tert-butyl (4-methoxy-5-(3-(methylamino) propyl)pyridin-2-yl)carbamate (A9) (1.56 g, 63% (2 steps)). ¹H NMR (400 MHz, CDCl₃) δ 7.87 (s, 1H), 7.83 (br s, 1H), 7.52 (s, 1H), 3.90 (s, 3H), 2.60-2.51 (m, 4H), 2.43 (s, 3H), 1.78-1.69 (m, 2H), 1.53 (s, 9H) ¹³C NMR (150 MHz, MeOD) δ 165.5, 153.2, 152.7, 147.0, 121.1, 94.8, 80.3, 54.5, 50.8, 34.6, 28.8, 27.2, 24.5 (3C); HR-MS (ESI): Calcd for C₁₅H₂₆N₃O₃⁺ [M+H]⁺; 296.1969; found, 296.1978.

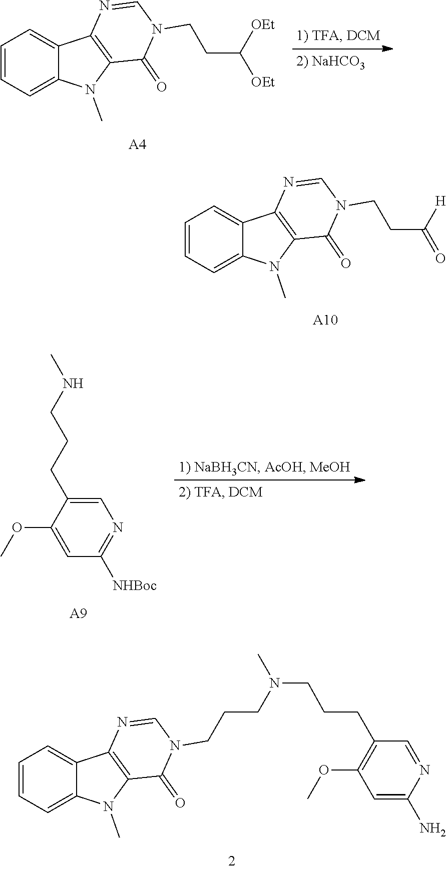

3-(3-((3-(6-amino-4-methoxypyridin-3-yl) propyl)(methyl)amino) propyl)-5-methyl-3,5-dihydro-4H-pyrimido[5,4-b]indol-4-one (2): A mixture of A4 (1.68 g, 5.10 mmol) in TFA/DCM (v/v, 25.7 mL) was stirred at room temperature for 30 min then concentrated in vacuo. Then, saturated NaHCO₃ solution (10 mL) was added, and the mixture was extracted with DCM (3×20 mL) and concentrated in vacuo to give A10. To this product, A9 (1.51 g, 5.10 mmol) was added in MeOH (51.0 mL), and the mixture was stirred at room temperature for 2 min. Subsequently NaBH₃CN (641 mg, 10.2 mmol) and AcOH (584 μL, 10.2 mmol) were added, and the mixture was stirred at room temperature overnight, whereupon saturated NaHCO₃ (20 mL) was added, and the mixture was extracted with DCM (3×20 mL) and concentrated in vacuo. The resultant material was dissolved in TFA/DCM (v/v, 25.7 mL) and the mixture was stirred at room temperature for 5 h, then concentrated in vacuo. The crude material was purified by reverse-phase column chromatography (C18 column, 120 g, 2%-100% MeOH+0.1% TFA/H₂O+0.1% TFA, 80 ml/min). The fractions were collected and neutralized with saturated NaHCO₃ and extracted with DCM (3×50 mL). The resultant organic layer was dried over MgSO₄ and concentrated in vacuo. The material was then purified by column chromatography (Biotage SNAP cartridge, KP-NH, 28 g, 100% ethyl acetate, 5 CV, 80 ml/min then 2%-20% MeOH in DCM, 5 CV, 80 ml/min) to afford 3-(3-((3-(6-amino-4-methoxypyridin-3-yl) propyl)(methyl)amino) propyl)-5-methyl-3,5-dihydro-4H-pyrimido[5,4-b]indol-4-one (2) (1.00 g, 45% (3 steps)). ¹H NMR (400 MHz, CDCl₃) δ 8.15 (d, J=8.0 Hz, 1H), 8.06 (s, 1H), 7.71 (s, 1H), 7.58-7.52 (m, 1H), 7.46 (d, J=8.4 Hz, 1H), 7.34-7.28 (m, 1H), 5.96 (s, 1H), 4.33 (br s, 2H), 4.26 (s, 3H), 4.16 (t, J=6.9 Hz, 2H), 3.79 (s, 3H), 2.47 (t, J=7.9 Hz, 2H), 2.42-2.32 (m, 4H), 2.21 (s, 3H), 2.03-1.95 (m, 2H), 1.75-1.65 (m, 2H) ¹³C NMR (150 MHz, CDCl₃) δ 165.4, 158.7, 155.5, 147.9, 143.7, 140.5, 138.2, 127.6, 121.9, 120.9, 120.9, 120.7, 117.6, 110.0, 90.3, 57.2, 54.9, 54.1, 44.7, 41.7, 31.3, 27.3, 26.8, 25.1; HR-MS (ESI): Calcd for C₂₄H₃₁N₆O₂⁺ [M+H]⁺; 435.2503; found, 435.2483.

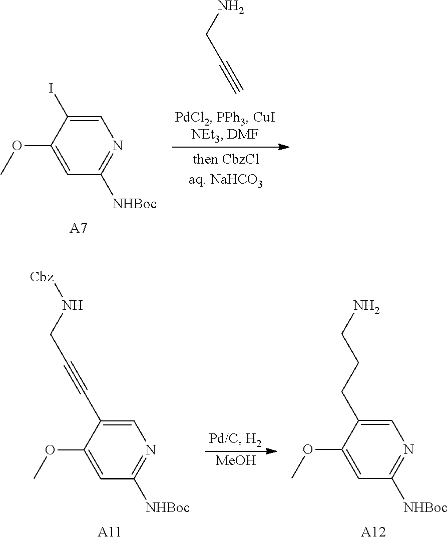

tert-butyl (4-methoxy-5-(3-(amino) propyl)pyridin-2-yl)carbamate (A12): A mixture of Ar-iodide A7 (1.50 g, 4.28 mmol), PdCl₂ (40.0 mg, 0.214 mmol, 5 mol %), PPh₃ (112 mg, 0.428 mmol, 10 mol %), CuI (81.6 mg, 0.428 mmol, 10 mol %), and propargylamine (823 mL, 12.9 mmol, 3 equiv.) in THF/DMF/Et₃N (10:3:3, 16 mL) was stirred at 50° C. for 2 h. After cooling to room temperature, saturated NaHCO₃ solution (20 mL) and CbzCl (3.00 mL, 21.4 mmol, 5 equiv.) were added, and the mixture was vigorously stirred for 3 h. The resulting mixture was passed through a Celite pad and the filtrate was extracted with AcOEt (3×150 mL). The combined organic layer was washed with brine (50 mL), dried over Na₂SO₄ and concentrated in vacuo. The crude product was roughly purified by column chromatography (Silica irregular 40-60 mm 80A 80 g, hexane/AcOEt 10-40%) to afford the coupling product A11 (850 mg, impure), which was used without further purification. A mixture of A11 (850 mg) and Pd/C (10%, 800 mg) in DCM/MeOH (v/v, 10 mL) was stirred at room temperature under H₂ (1 atm) for 1 h. The reaction mixture was filtered through a Celite pad and the filtrate was concentrated in vacuo. The crude product was purified by column chromatography (Sfär KP-Amino D 28 g, DCM/MeOH 0-20%) to afford the title amine A12 (402 mg, 34% in 2 steps). ¹H NMR (400 MHz, CDCl₃) δ 7.89 (s, 1H), 7.54 (s, 1H), 3.90 (s, 3H), 2.70 (t, J=7.2 Hz, 2H), 2.55 (t, J=7.4 Hz, 2H), 1.73-1.66 (m, 2H), 1.53 (s, 9H); ¹³C NMR (150 MHz, CDCl₃) δ 165.3, 152.7, 152.4, 147.6, 121.2, 94.5, 80.7, 55.3, 41.8, 33.7, 28.4, 24.6; HR-MS (ESI): Calcd for C₁₄H₂₄N₃O₃⁺ [M+H]⁺; 282.1812; found, 282.1776.

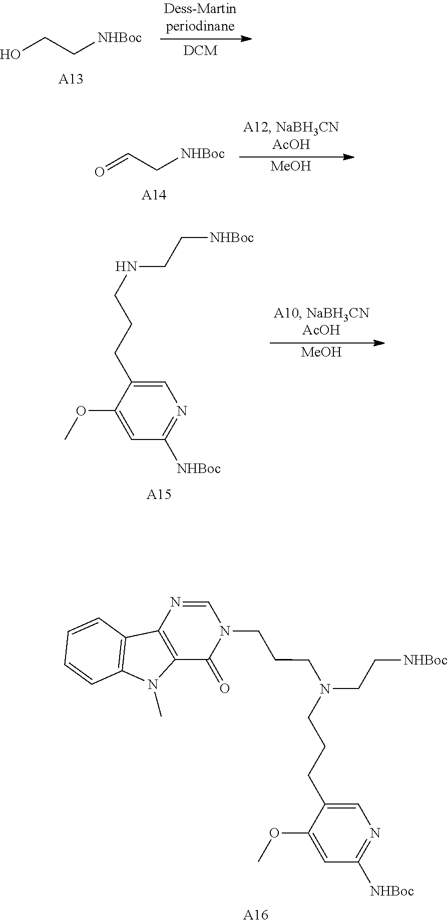

tert-butyl (5-(3-((2-((tert-butoxycarbonyl)amino)ethyl)amino) propyl)-4-methoxypyridin-2-yl)carbamate) (A15): Dess-Martin periodinane (325 mg, 0.768 mmol) was added to a solution of A13 (70.0 mg, 0.427 mmol) in DCM (5 mL), and the mixture was stirred for 1 h. The reaction mixture was quenched by 10% aqueous Na₂S203 (3 mL) and saturated NaHCO₃ solution (3 mL). The organic layer was extracted by DCM (10 mL×2), dried over Na₂SO₄, and concentrated in vacuo. The crude aldehyde A14 was used without further purification. AcOH (30.5 mL, 0.533 mmol, 1.5 equiv.) was added to a mixture of crude A14 and amine A12 (100 mg, 0.355 mmol) in MeOH, and the mixture was stirred for 30 min. After NaBH₃CN (67.0 mg, 1.07 mmol, 3 equiv.) was added, the reaction mixture was stirred for 2 d. To the resulting mixture, sat. aqueous NaHCO₃ was added, and the organic layer was extracted with DCM, dried over Na₂SO₄, and concentrated in vacuo. The crude product was purified by column chromatography (Sfär KP-Amino D 11 g, 0-5% DCM/MeOH) to give the A15 (55.1 mg, 37%). ¹H NMR (400 MHz, CDCl₃) δ 8.59 (br s, 1H), 7.90 (s, 1H), 7.55 (s, 1H), 4.98 (br s, 1H), 3.90 (s, 3H), 3.22 (m, 2H), 2.71 (t, J=7.3 Hz, 2H), 2.60 (t, J=8.9 Hz, 2H), 2.54 (t, J=8.9 Hz, 2H), 1.75-1.68 (m, 2H), 1.54 (s, 9H), 1.44 (s, 9H); ¹³C NMR (150 MHz, CDCl₃) δ 165.3, 156.1, 152.8, 152.6, 147.5, 121.0, 94.6, 80.7, 79.2, 55.3, 49.5, 49.1, 40.4, 30.0, 28.4, 28.4, 25.0; HR-MS (ESI): Calcd for C₂₁H₃₇N₄O₅⁺ [M+H]⁺; 425.2758; found, 425.2780.

tert-butyl (5-(3-((2-((tert-butoxycarbonyl)amino)ethyl) (3-(5-methyl-4-oxo-4,5-dihydro-3H-pyrimido[5,4-b]indol-3-yl) propyl)amino) propyl)-4-methoxypyridin-2-yl)carbamate (A16): AcOH (1 μL, 17 mmol, 0.1 equiv.) was added to a mixture of the crude aldehyde A10 and amine A15 (85.4 mg, 0.201 mmol), and the mixture was stirred at room temperature for 30 min. Subsequently, NaBH3CN (102 mg, 1.61 mmol, 9 equiv.) was added, and the mixture was stirred at room temperature overnight, whereupon sat. aqueous NaHCO₃ (5 mL) was added, and the organic layer was extracted with DCM (10 mL×3). The combined organic layer was dried over Na₂SO₄, and concentrated in vacuo. The crude product was purified by column chromatography (Sfär KP-Amino D 28 g, 30-75% hexane/AcOEt) to afford the amine A16 (44.6 mg, 35%). ¹H NMR (400 MHz, CDCl₃) δ 9.01 (s, 1H), 8.15 (d, J=7.9 Hz, 1H), 8.01 (s, 1H), 7.86 (s, 1H), 7.75 (bs, 1H), 7.58-7.53 (m, 1H), 7.51 (s, 1H), 7.46 (d, J=7.9 Hz, 1H), 7.33-7.29 (m, 1H), 5.07 (br s, 1H), 4.26 (s, 3H), 4.15-4.10 (m, 1H), 3.89 (s, 3H), 3.19 (m, 2H), 2.55-2.47 (m, 6H), 1.99-1.94 (m, 2H), 1.73-1.63 (m, 4H), 1.53 (s, 9H), 1.43 (m, 9H); ¹³C NMR (150 MHz, CDCl₃) δ 165.3, 156.2, 155.5, 153.0, 152.9, 147.5, 143.3, 140.5, 138.2, 127.7, 121.9, 121.0, 121.0, 120.8, 110.1, 94.7, 80.7, 55.4, 53.7, 53.5, 53.3, 51.1, 45.0, 31.4, 28.5, 27.4, 27.0, 25.5; HR-MS (ESI): Calcd for C₃₅H₅₀N₇O₆⁺ [M+H]⁺; 664.3817; found, 664.3845.

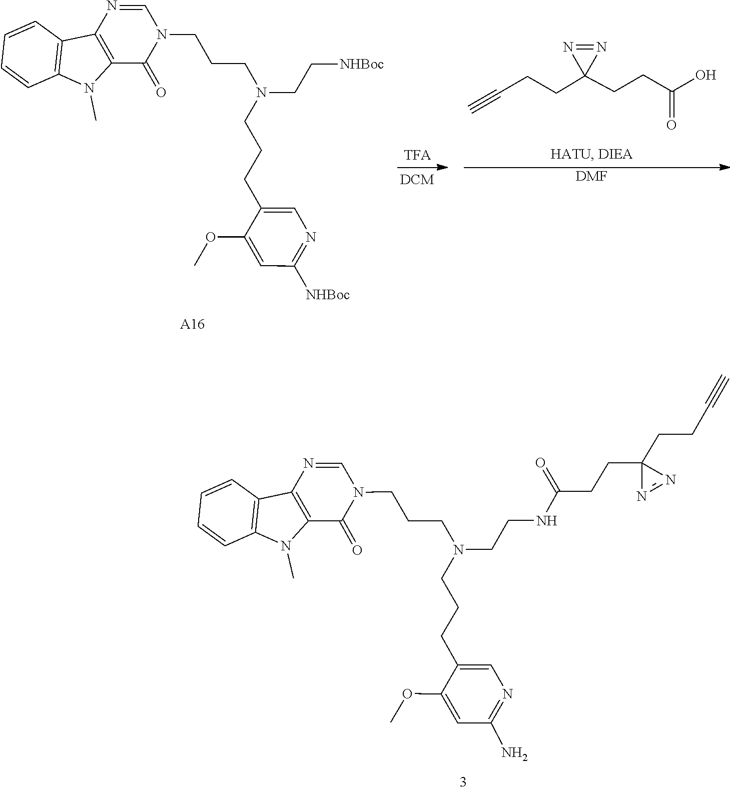

N-(2-((3-(6-amino-4-methoxypyridin-3-yl) propyl) (3-(5-methyl-4-oxo-4,5-dihydro-3H-pyrimido[5,4-b]indol-3-yl) propyl)amino)ethyl)-3-(3-(but-3-yn-1-yl)-3H-diazirin-3-yl) propenamide (3): A mixture of Boc-amine A16 (8.6 mg, 13.6 μmol) in DCM/TFA (v/v, 1 mL) was stirred for 2 h, and the mixture was concentrated in vacuo to give the crude product, which was used without further purification. Diazirine carboxylic acid (6.6 mg, 43 μmol) was pre-activated with HATU (18.6 mg, 49 μmol) and DIEA (15.7 mL, 98 μmol) in DMF (450 μL). To a solution of the crude amine and DIEA (6.5 mL, 41 μmol) in DMF (150 μL) the pre-activated solution (150 μL) was added, and the mixture was stirred for 4 h. An additional portion of pre-activated solution (50 μL) was added to the mixture, and the mixture was stirred overnight. The resulting mixture was diluted with MeOH, and directly purified by HPLC (SunFire Prep C18 OBD 5 μm, 18×150 mm, 20-100% MeOH/H₂O with 0.1% TFA for 60 min) to afford diazirine alkyne 3 (7.2 mg, 86%). ¹H NMR (400 MHz, MeOD) δ 8.30 (s, 1H), 8.10 (d, J=8.0 Hz, 1H), 7.63-7.57 (m, 3H), 7.34-7.30 (m, 1H), 6.35 (s, 1H), 4.26 (t, J=6.6 Hz, 2H), 4.23 (s, 3H), 3.57 (br, 2H), 3.37-3.35 (m, 6H), 2.57 (t, J=7.2 Hz, 2H), 2.31-2.27 (m, 2H), 2.25 (t, J=2.64 Hz, 1H), 2.07 (t, J=7.3 Hz, 2H), 2.00-1.92 (m, 4H), 1.73 (t, J=7.5 Hz, 2H), 1.54 (t, J=7.4 Hz, 2H); ¹³C NMR (150 MHz, MeOD) δ 176.4, 170.9, 157.3, 157.1, 145.3, 142.3, 139.3, 135.5, 129.5, 122.3, 121.8, 121.7, 118.6, 111.6, 93.0, 83.7, 70.5, 57.7, 54.7, 54.2, 52.0, 44.6, 36.1, 33.4, 32.9, 31.9, 30.8, 29.3, 25.6, 24.8, 24.3, 13.9; HR-MS (ESI): Calcd for C₃₃H₄₂N₉O₃⁺ [M+H]⁺; 612.3405; found, 612.3402.

### REFERENCES (EXAMPLES 2 AND 3)

1. Delaglio, F.; Grzesiek, S.; Vuister, G. W.; Zhu, G.; Pfeifer, J.; Bax, A., NMRPipe: A multidimensional spectral processing system based on UNIX pipes. Journal of Biomolecular NMR 1995, 6 (3), 277-293.
2. Lee, W.; Tonelli, M.; Markley, J. L., NMRFAM-SPARKY: enhanced software for biomolecular NMR spectroscopy. Bioinformatics 2015, 31 (8), 1325-1327.
3. Case, D.; Ben-Shalom, I.; Brozell, S.; Cerutti, D.; Cheatham III, T.; Cruzeiro, V.; Darden, T.; Duke, R.; Ghoreishi, D.; Gilson, M., AMBER 2018; 2018. University of California, San Francisco 2018.
4. Cornell, W. D.; Cieplak, P.; Bayly, C. I.; Gould, I. R.; Merz, K. M.; Ferguson, D. M.; Spellmeyer, D. C.; Fox, T.; Caldwell, J. W.; Kollman, P. A., A Second Generation Force Field for the Simulation of Proteins, Nucleic Acids, and Organic Molecules. Journal of the American Chemical Society 1995, 117 (19), 5179-5197.
5 Yildirim, I.; Stern, H. A.; Kennedy, S. D.; Tubbs, J. D.; Turner, D. H., Reparameterization of RNA chi Torsion Parameters for the AMBER Force Field and Comparison to NMR Spectra for Cytidine and Uridine. J Chem Theory Comput 2010, 6 (5), 1520-1531.
6. Wales, D. J.; Yildirim, I., Improving Computational Predictions of Single-Stranded RNA Tetramers with Revised alpha/gamma Torsional Parameters for the Amber Force Field. J Phys Chem B 2017, 121 (14), 2989-2999.
7. Joung, I. S.; Cheatham, T. E., 3rd, Determination of alkali and halide monovalent ion parameters for use in explicitly solvated biomolecular simulations. J Phys Chem B 2008, 112 (30), 9020-41.
8. Jorgensen, W. L.; Chandrasekhar, J.; Madura, J. D.; Impey, R. W.; Klein, M. L., Comparison of simple potential functions for simulating liquid water. The Journal of Chemical Physics 1983, 79 (2), 926-935.
9. Liu, J.; Li, D.; Liu, X., A simple and accurate algorithm for path integral molecular dynamics with the Langevin thermostat. The Journal of Chemical Physics 2016, 145 (2), 024103.
10. Berendsen, H. J. C.; Postma, J. P. M.; Vangunsteren, W. F.; Dinola, A.; Haak, J. R., Molecular-Dynamics with Coupling to an External Bath. Journal of Chemical Physics 1984, 81 (8), 3684-3690.
11. Ryckaert, J. P.; Ciccotti, G.; Berendsen, H. J. C., Numerical-Integration of Cartesian Equations of Motion of a System with Constraints-Molecular-Dynamics of N-Alkanes. Journal of Computational Physics 1977, 23 (3), 327-341.
12. Essmann, U.; Perera, L.; Berkowitz, M. L.; Darden, T.; Lee, H.; Pedersen, L. G., A smooth particle mesh Ewald method. The Journal of Chemical Physics 1995, 103 (19), 8577-8593.
13. Buneman, O., Computer Simulation Using Particles (R. W. Hockney and J. W. Eastwood). SIAM Review 1983, 25 (3), 425-426.
14. Bayly, C. I.; Cieplak, P.; Cornell, W.; Kollman, P. A., A well-behaved electrostatic potential based method using charge restraints for deriving atomic charges: the RESP model. The Journal of Physical Chemistry 1993, 97 (40), 10269-10280.
15. Wang, J.; Wolf, R. M.; Caldwell, J. W.; Kollman, P. A.; Case, D. A., Development and testing of a general amber force field. J Comput Chem 2004, 25 (9), 1157-74.
16. Wang, J.; Wang, W.; Kollman, P. A.; Case, D. A., Automatic atom type and bond type perception in molecular mechanical calculations. J Mol Graph Model 2006, 25 (2), 247-60.
17. Frisch, M.; Trucks, G.; Schlegel, H.; Scuseria, G.; Robb, M.; Cheeseman, J.; Scalmani, G.; Barone, V.; Mennucci, B.; Petersson, G., Gaussian 09, Revision D. 01, Gaussian, Inc., Wallingford C T. See also: URL: http://www.gaussian.com 2009.
18. Chen, J. L.; Zhang, P.; Abe, M.; Aikawa, H.; Zhang, L.; Frank, A. J.; Zembryski, T.; Hubbs, C.; Park, H.; Withka, J.; Steppan, C.; Rogers, L.; Cabral, S.; Pettersson, M.; Wager, T. T.; Fountain, M. A.; Rumbaugh, G.; Childs-Disney, J. L.; Disney, M. D., Design, Optimization, and Study of Small Molecules That Target Tau Pre-mRNA and Affect Splicing. J Am Chem Soc 2020, 142 (19), 8706-8727.
19. Trott, O.; Olson, A. J., AutoDock Vina: improving the speed and accuracy of docking with a new scoring function, efficient optimization, and multithreading. J Comput Chem 2010, 31 (2), 455-61.
20. O'Boyle, N. M.; Banck, M.; James, C. A.; Morley, C.; Vandermeersch, T.; Hutchison, G. R., Open Babel: An open chemical toolbox. J Cheminform 2011, 3, 33.
21. Gasteiger, J.; Marsili, M., Iterative partial equalization of orbital electronegativity-a rapid access to atomic charges. Tetrahedron 1980, 36 (22), 3219-3228.
22. Santos-Martins, D.; Solis-Vasquez, L.; Tillack, A. F.; Sanner, M. F.; Koch, A.; Forli, S., Accelerating AutoDock4 with GPUs and Gradient-Based Local Search. J Chem Theory Comput 2021, 17 (2), 1060-1073.
23. Solis, F. J.; Wets, R. J. B., Minimization by Random Search Techniques. Mathematics of Operations Research 1981, 6 (1), 19-30.
24. Ribeiro, J.; Rios-Vera, C.; Melo, F.; Schuller, A., Calculation of accurate interatomic contact surface areas for the quantitative analysis of non-bonded molecular interactions. Bioinformatics 2019, 35 (18), 3499-3501.
25. Szulc, N. A.; Mackiewicz, Z.; Bujnicki, J. M.; Stefaniak, F., fingeRNAt-A novel tool for high-throughput analysis of nucleic acid-ligand interactions. PLOS Comput Biol 2022, 18 (6), e1009783.
26. Kovalenko, A.; Hirata, F., Three-dimensional density profiles of water in contact with a solute of arbitrary shape: a RISM approach. Chemical Physics Letters 1998, 290 (1), 237-244.
27. Luchko, T.; Gusarov, S.; Roe, D. R.; Simmerling, C.; Case, D. A.; Tuszynski, J.; Kovalenko, A., Three-Dimensional Molecular Theory of Solvation Coupled with Molecular Dynamics in Amber. Journal of Chemical Theory and Computation 2010, 6 (3), 607-624.
28. Johnson, J.; Case, D.; Yamazaki, T.; Gusarov, S.; Kovalenko, A.; Luchko, T., Small molecule hydration energy and entropy from 3D-RISM. Journal of Physics: Condensed Matter 2016, 28 (34), 344002.
29. Gusarov, S.; Ziegler, T.; Kovalenko, A., Self-consistent combination of the three-dimensional RISM theory of molecular solvation with analytical gradients and the Amsterdam density functional package. J Phys Chem A 2006, 110 (18), 6083-90.
30. Velagapudi, S. P.; Luo, Y.; Tran, T.; Haniff, H. S.; Nakai, Y.; Fallahi, M.; Martinez, G. J.; Childs-Disney, J. L.; Disney, M. D., Defining RNA-Small Molecule Affinity Landscapes Enables Design of a Small Molecule Inhibitor of an Oncogenic Noncoding RNA. ACS Cent Sci 2017, 3 (3), 205-216.
31. McDowell, J. A.; Turner, D. H., Investigation of the structural basis for thermodynamic stabilities of tandem GU mismatches: solution structure of (rGAGGUCUC) 2 by two-dimensional NMR and simulated annealing. Biochemistry 1996, 35 (45), 14077-89.
32. Velagapudi, S. P.; Li, Y.; Disney, M. D., A cross-linking approach to map small molecule-RNA binding sites in cells. Bioorg Med Chem Lett 2019, 29 (12), 1532-1536.
33. Lee, K.-Y.; Seah, C.; Li, C.; Chen, Y.-F.; Chen, C.-Y.; Wu, C.-I.; Liao, P.-C.; Shyu, Y.-C.; Olafson, H. R.; McKee, K. K.; Wang, E. T.; Yeh, C.-H.; Wang, C.-H., Mice lacking MBNL1 and MBNL2 exhibit sudden cardiac death and molecular signatures recapitulating myotonic dystrophy. Human Molecular Genetics 2022, 31 (18), 3144-3160.
34. Andrews, S., FastQC: a quality control tool for high throughput sequence data. Babraham Bioinformatics, Babraham Institute, Cambridge, United Kingdom: 2010.
35. Wang, L.; Wang, S.; Li, W., RSeQC: quality control of RNA-seq experiments. Bioinformatics 2012, 28 (16), 2184-2185.
36. Kim, D.; Paggi, J. M.; Park, C.; Bennett, C.; Salzberg, S. L., Graph-based genome alignment and genotyping with HISAT2 and HISAT-genotype. Nature Biotechnology 2019, 37 (8), 907-915.
37. Bray, N. L.; Pimentel, H.; Melsted, P.; Pachter, L., Near-optimal probabilistic RNA-seq quantification. Nature Biotechnology 2016, 34 (5), 525-527.
38. O'Leary, N. A.; Wright, M. W.; Brister, J. R.; Ciufo, S.; Haddad, D.; Mcveigh, R.; Rajput, B.; Robbertse, B.; Smith-White, B.; Ako-Adjei, D.; Astashyn, A.; Badretdin, A.; Bao, Y.; Blinkova, O.; Brover, V.; Chetvernin, V.; Choi, J.; Cox, E.; Ermolaeva, O.; Farrell, C. M.; Goldfarb, T.; Gupta, T.; Haft, D.; Hatcher, E.; Hlavina, W.; Joardar, V. S.; Kodali, V. K.; Li, W.; Maglott, D.; Masterson, P.; McGarvey, K. M.; Murphy, M. R.; O'Neill, K.; Pujar, S.; Rangwala, S. H.; Rausch, D.; Riddick, L. D.; Schoch, C.; Shkeda, A.; Storz, S. S.; Sun, H.; Thibaud-Nissen, F.; Tolstoy, I.; Tully, R. E.; Vatsan, A. R.; Wallin, C.; Webb, D.; Wu, W.; Landrum, M. J.; Kimchi, A.; Tatusova, T.; DiCuccio, M.; Kitts, P.; Murphy, T. D.; Pruitt, K. D., Reference sequence (RefSeq) database at NCBI: current status, taxonomic expansion, and functional annotation. Nucleic Acids Research 2016, 44 (D1), D733-D745.
39. Pimentel, H.; Bray, N. L.; Puente, S.; Melsted, P.; Pachter, L., Differential analysis of RNA-seq incorporating quantification uncertainty. Nature Methods 2017, 14 (7), 687-690.
40. Katz, Y.; Wang, E. T.; Airoldi, E. M.; Burge, C. B., Analysis and design of RNA sequencing experiments for identifying isoform regulation. Nature Methods 2010, 7 (12), 1009-1015.
41. Rumbaugh, G.; Sillivan, S. E.; Ozkan, E. D.; Rojas, C. S.; Hubbs, C. R.; Aceti, M.; Kilgore, M.; Kudugunti, S.; Puthanveettil, S. V.; Sweatt, J. D.; Rusche, J.; Miller, C. A., Pharmacological Selectivity Within Class I Histone Deacetylases Predicts Effects on Synaptic Function and Memory Rescue. Neuropsychopharmacology 2015, 40 (10), 2307-2316.
42. Polydoro, M.; Acker, C. M.; Duff, K.; Castillo, P. E.; Davies, P., Age-dependent impairment of cognitive and synaptic function in the htau mouse model of tau pathology. J Neurosci 2009, 29 (34), 10741-9.
43. Deacon, R. M., Assessing nest building in mice. Nat Protoc 2006, 1 (3), 1117-9.
44. Donahue, C. P.; Muratore, C.; Wu, J. Y.; Kosik, K. S.; Wolfe, M. S., Stabilization of the tau exon 10 stem loop alters pre-mRNA splicing. J Biol Chem 2006, 281 (33), 23302-6.
45. Wishart, D. S.; Feunang, Y. D.; Guo, A. C.; Lo, E. J.; Marcu, A.; Grant, J. R.; Sajed, T.; Johnson, D.; Li, C.; Sayeeda, Z.; Assempour, N.; lynkkaran, I.; Liu, Y.; Maciejewski, A.; Gale, N.; Wilson, A.; Chin, L.; Cummings, R.; Le, D.; Pon, A.; Knox, C.; Wilson, M., DrugBank 5.0: a major update to the DrugBank database for 2018. Nucleic Acids Research 2018, 46 (D1), D1074-D1082.

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

What is claimed is:

1. A compound of Formula (I): 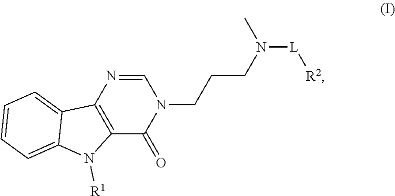
or a pharmaceutically acceptable salt, solvate, hydrate, polymorph, co-crystal, tautomer, stereoisomer, isotopically labeled compound, or prodrug thereof, wherein:
L is a linker;
R¹ is hydrogen or optionally substituted alkyl; and
R² is optionally substituted heteroaryl.

2. The compound of claim 1, wherein the compound is of Formula (I-a-i): 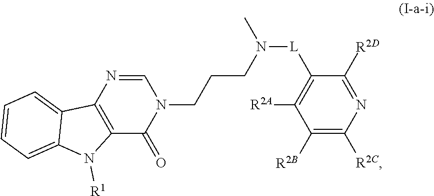
or a pharmaceutically acceptable salt, solvate, hydrate, polymorph, co-crystal, tautomer, stereoisomer, isotopically labeled compound, or prodrug thereof, wherein:
each of R^{2A}, R^{2B}, R^{2C}, and R^{2D} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; and
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom or atoms to form an optionally substituted heterocyclic ring or optionally substituted heteroaryl ring.

3. The compound of claim 2, or a pharmaceutically acceptable salt, solvate, hydrate, polymorph, co-crystal, tautomer, stereoisomer, isotopically labeled compound, or prodrug thereof, wherein at least one of R^{2A}, R^{2B}, R^{2C}, and R^{2D} is hydrogen, —OR^{A}, or —N(R^{A})₂.

4. The compound of any one of claim 2 or 3, or a pharmaceutically acceptable salt, solvate, hydrate, polymorph, co-crystal, tautomer, stereoisomer, isotopically labeled compound, or prodrug thereof, wherein each of R^{2A}, R^{2B}, R^{2C}, and R^{2D} is independently hydrogen, —OR^{A}, or —N(R^{A})₂.

5. The compound of any one of claims 2-4, or a pharmaceutically acceptable salt, solvate, hydrate, polymorph, co-crystal, tautomer, stereoisomer, isotopically labeled compound, or prodrug thereof, wherein at least one of R^{2A}, R^{2B}, R^{2C}, and R^{2D} is hydrogen.

6. The compound of any one of claims 2-5, or a pharmaceutically acceptable salt, solvate, hydrate, polymorph, co-crystal, tautomer, stereoisomer, isotopically labeled compound, or prodrug thereof, wherein at least one of R^{2A}, R^{2B}, R^{2C}, and R^{2D} is —OR^{A}.

7. The compound of any one of claims 2-6, or a pharmaceutically acceptable salt, solvate, hydrate, polymorph, co-crystal, tautomer, stereoisomer, isotopically labeled compound, or prodrug thereof, wherein at least one of R^{2A}, R^{2B}, R^{2C}, and R^{2D} is —N(R^{A})₂.

8. The compound of any one of claims 2-7, wherein the compound is of Formula (I-a-ii): 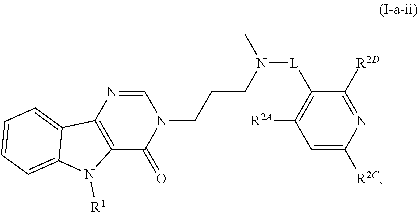
or a pharmaceutically acceptable salt, solvate, hydrate, polymorph, co-crystal, tautomer, stereoisomer, isotopically labeled compound, or prodrug thereof.

9. The compound of any one of claims 2-7, wherein the compound is of Formula (I-a-iii): 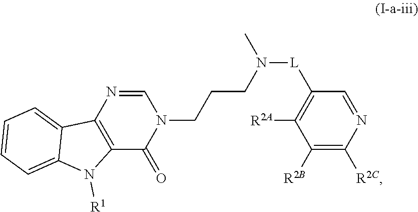
or a pharmaceutically acceptable salt, solvate, hydrate, polymorph, co-crystal, tautomer, stereoisomer, isotopically labeled compound, or prodrug thereof.

10. The compound of any one of claims 2-9, wherein the compound is of Formula (I-a-iv): 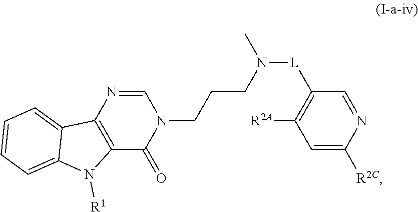
or a pharmaceutically acceptable salt, solvate, hydrate, polymorph, co-crystal, tautomer, stereoisomer, isotopically labeled compound, or prodrug thereof.

11. The compound of any one of claims 2-10, or a pharmaceutically acceptable salt, solvate, hydrate, polymorph, co-crystal, tautomer, stereoisomer, isotopically labeled compound, or prodrug thereof, wherein R^{2A} is —OR^{A}, and R^{A} is hydrogen or optionally substituted alkyl.

12. The compound of any one of claims 2-11, or a pharmaceutically acceptable salt, solvate, hydrate, polymorph, co-crystal, tautomer, stereoisomer, isotopically labeled compound, or prodrug thereof, wherein R^{2C} is —N(R^{A})₂, and each occurrence of R^{A} is independently hydrogen or optionally substituted alkyl.

13. The compound of any one of claims 2-12, wherein the compound is of Formula (I-a-v): 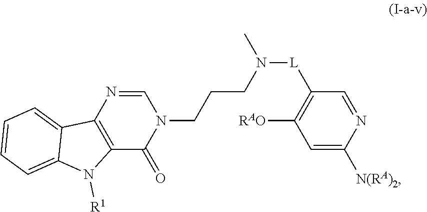
or a pharmaceutically acceptable salt, solvate, hydrate, polymorph, co-crystal, tautomer, stereoisomer, isotopically labeled compound, or prodrug thereof.

14. The compound of any one of claims 2-13, or a pharmaceutically acceptable salt, solvate, hydrate, polymorph, co-crystal, tautomer, stereoisomer, isotopically labeled compound, or prodrug thereof, wherein R^{2A} is —OCH₃.

15. The compound of any one of claims 2-14, or a pharmaceutically acceptable salt, solvate, hydrate, polymorph, co-crystal, tautomer, stereoisomer, isotopically labeled compound, or prodrug thereof, wherein R^{2C} is —NH₂.

16. The compound of any one of claims 2-14, or a pharmaceutically acceptable salt, solvate, hydrate, polymorph, co-crystal, tautomer, stereoisomer, isotopically labeled compound, or prodrug thereof, wherein R^{2C} is —NHCH₃.

17. The compound of any one of claims 1-16, or a pharmaceutically acceptable salt, solvate, hydrate, polymorph, co-crystal, tautomer, stereoisomer, isotopically labeled compound, or prodrug thereof, wherein R¹ is hydrogen.

18. The compound of any one of claims 1-16, or a pharmaceutically acceptable salt, solvate, hydrate, polymorph, co-crystal, tautomer, stereoisomer, isotopically labeled compound, or prodrug thereof, wherein R¹ is optionally substituted alkyl.

19. The compound of any one of claim 1-16 or 18, or a pharmaceutically acceptable salt, solvate, hydrate, polymorph, co-crystal, tautomer, stereoisomer, isotopically labeled compound, or prodrug thereof, wherein R¹ is —CH₃.

20. The compound of any one of claims 1-19, or a pharmaceutically acceptable salt, solvate, hydrate, polymorph, co-crystal, tautomer, stereoisomer, isotopically labeled compound, or prodrug thereof, wherein L is optionally substituted alkylene.

21. The compound of any one of claims 1-20, or a pharmaceutically acceptable salt, solvate, hydrate, polymorph, co-crystal, tautomer, stereoisomer, isotopically labeled compound, or prodrug thereof, wherein L is optionally substituted C₁-C₆ alkylene.

22. The compound of any one of claim 1-7, 11, 12, or 14-21, wherein the compound is of Formula (I-b-i): 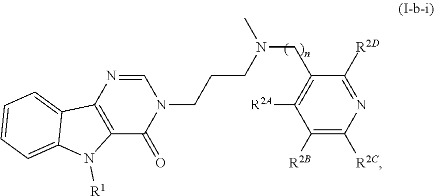
or a pharmaceutically acceptable salt, solvate, hydrate, polymorph, co-crystal, tautomer, stereoisomer, isotopically labeled compound, or prodrug thereof, wherein:
each of R^{2A}, R^{2B}, R^{2C}, and R^{2D} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom or atoms to form an optionally substituted heterocyclic ring or optionally substituted heteroaryl ring; and
n is 1, 2, 3, 4, or 5.

23. The compound of claim 22, or a pharmaceutically acceptable salt, solvate, hydrate, polymorph, co-crystal, tautomer, stereoisomer, isotopically labeled compound, or prodrug thereof, wherein n is 3.

24. The compound of any one of claim 1-7, 11, 12, or 14-22, wherein the compound is of Formula (I-b-ii): 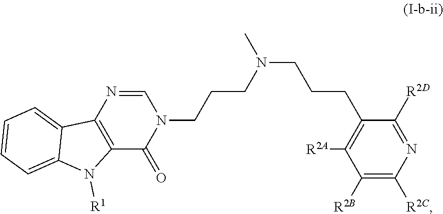
or a pharmaceutically acceptable salt, solvate, hydrate, polymorph, co-crystal, tautomer, stereoisomer, isotopically labeled compound, or prodrug thereof.

25. The compound of any one of claim 1-7, 11, 12, or 14-21, wherein the compound is of Formula (I-b-iii): 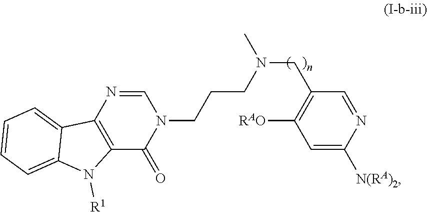
or a pharmaceutically acceptable salt, solvate, hydrate, polymorph, co-crystal, tautomer, stereoisomer, isotopically labeled compound, or prodrug thereof.

26. The compound of any one of claims 1-25, wherein the compound is of Formula (I-b-vi): 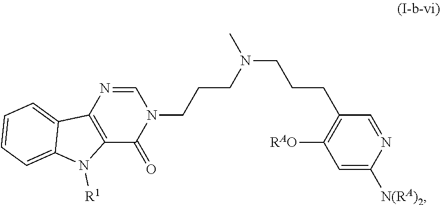
or a pharmaceutically acceptable salt, solvate, hydrate, polymorph, co-crystal, tautomer, stereoisomer, isotopically labeled compound, or prodrug thereof.

27. The compound of any one of claims 1-26, wherein the compound is of formula: 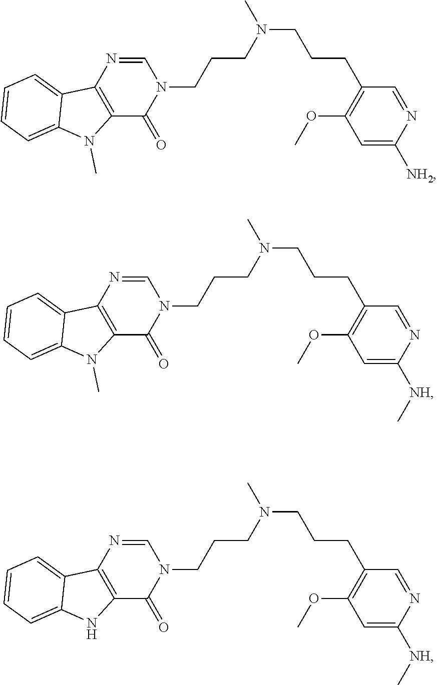 [SMILES: COc1cc(N)ncc1CCCN(C)CCCn1cnc2c3ccccc3n(C)c2c1=O ; CNc1cc(OC)c(CCCN(C)CCCn2cnc3c4ccccc4n(C)c3c2=O)cn1 ; CNc1cc(OC)c(CCCN(C)CCCn2cnc3c([nH]c4ccccc43)c2=O)cn1]
or a pharmaceutically acceptable salt, solvate, hydrate, polymorph, co-crystal, tautomer, stereoisomer, isotopically labeled compound, or prodrug thereof.

28. A compound of formula: 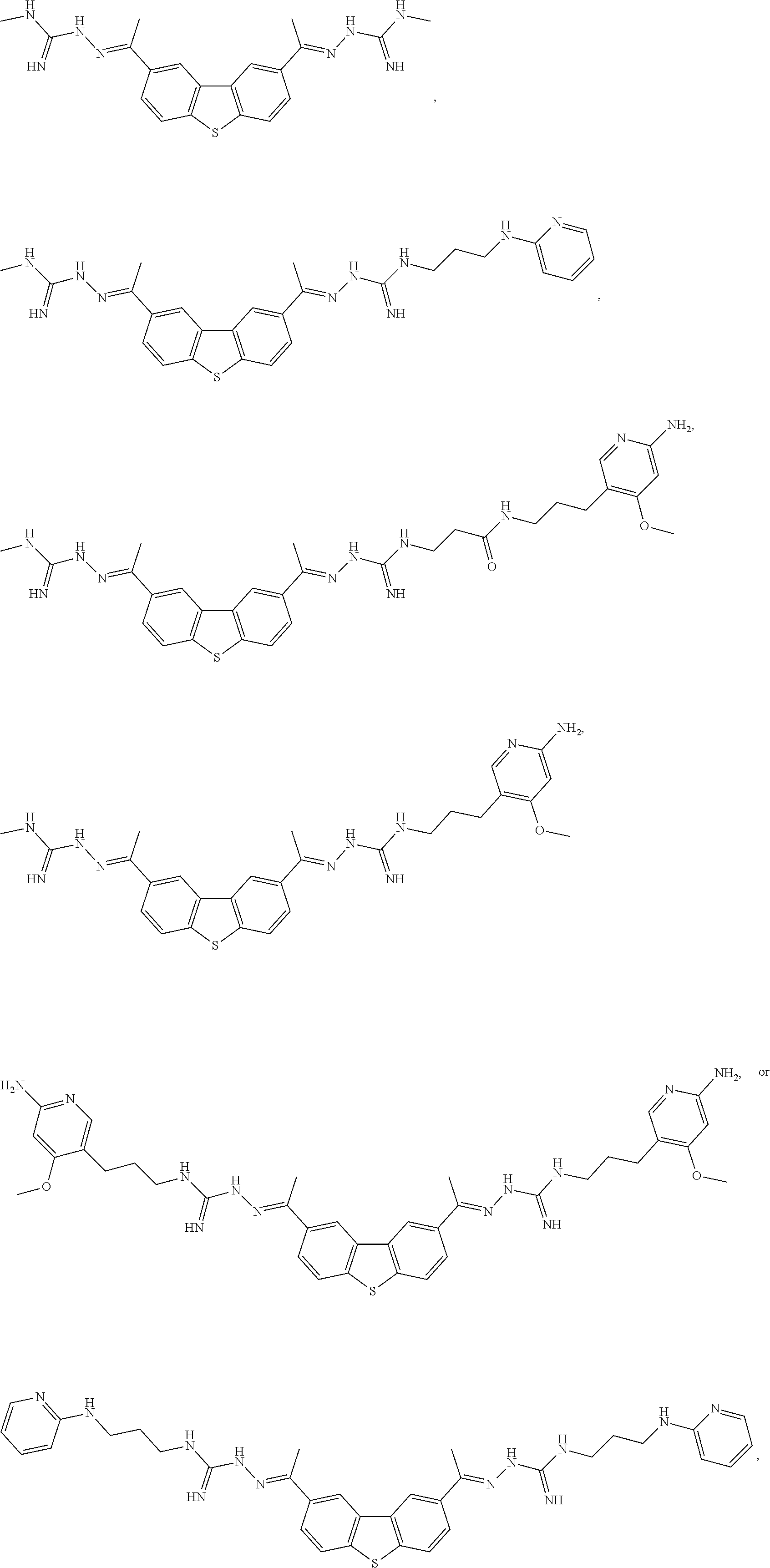
or a pharmaceutically acceptable salt, solvate, hydrate, polymorph, co-crystal, tautomer, stereoisomer, isotopically labeled compound, or prodrug thereof.

29. A compound of formula: 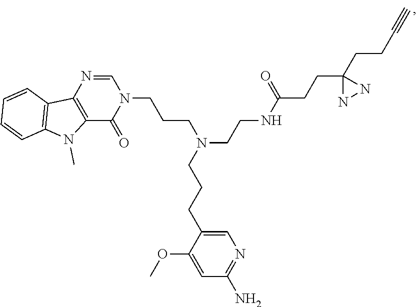 [SMILES: C#CCCC1(CCC(=O)NCCN(CCCc2cnc(N)cc2OC)CCCn2cnc3c4ccccc4n(C)c3c2=O)N=N1]
or a pharmaceutically acceptable salt, solvate, hydrate, polymorph, co-crystal, tautomer, stereoisomer, isotopically labeled compound, or prodrug thereof.

30. A composition comprising the compound of any one of claims 1-29, or a pharmaceutically acceptable salt, solvate, hydrate, polymorph, co-crystal, tautomer, stereoisomer, isotopically labeled compound, or prodrug thereof, and an excipient.

31. A method of stabilizing an RNA target in a subject in need thereof or in a cell, tissue, or biological sample, comprising administering to the subject in need thereof or contacting the cell, tissue, or biological sample with an effective amount of:
a compound of any one of claims 1-29, or a pharmaceutically acceptable salt, solvate, hydrate, polymorph, co-crystal, tautomer, stereoisomer, isotopically labeled compound, or prodrug thereof; or
the composition of claim 30.

32. The compound of any one of claims 1-29, or a pharmaceutically acceptable salt, solvate, hydrate, polymorph, co-crystal, tautomer, stereoisomer, isotopically labeled compound, or prodrug thereof, or the composition of claim 30, for use in stabilizing an RNA target in a subject in need thereof or in a cell, tissue, or biological sample.

33. The compound of any one of claims 1-29, or a pharmaceutically acceptable salt, solvate, hydrate, polymorph, co-crystal, tautomer, stereoisomer, isotopically labeled compound, or prodrug thereof, or the composition of claim 30, for use in the manufacture of a medicament for stabilizing an RNA target in a subject in need thereof or in a cell, tissue, or biological sample.

34. The method, compound for use, pharmaceutically acceptable salt, solvate, hydrate, polymorph, co-crystal, tautomer, stereoisomer, isotopically labeled compound, or prodrug thereof for use, or composition for use of any one of claims 31-33, wherein the RNA target is microtubule-associated protein Tau (MAPT) pre-mRNA.

35. The method, compound for use, pharmaceutically acceptable salt, solvate, hydrate, polymorph, co-crystal, tautomer, stereoisomer, isotopically labeled compound, or prodrug thereof for use, or composition for use of claim 34, comprising intercalating to an A bulge site of the MAPT pre-MRNA.

36. The method, compound for use, pharmaceutically acceptable salt, solvate, hydrate, polymorph, co-crystal, tautomer, stereoisomer, isotopically labeled compound, or prodrug thereof for use, or composition for use of any one of claim 34 or 35, comprising forming a base triple with a GC pair of the MAPT pre-MRNA.

37. The method, compound for use, pharmaceutically acceptable salt, solvate, hydrate, polymorph, co-crystal, tautomer, stereoisomer, isotopically labeled compound, or prodrug thereof for use, or composition for use of any one of claims 31-36, comprising inducing exon 10 skipping.

38. A method of decreasing a ratio of an amount of a first mRNA isoform to an amount of a second mRNA isoform in a subject in need thereof or in a cell, tissue, or biological sample, comprising administering to the subject in need thereof or contacting the cell, tissue, or biological sample with an effective amount of:
a compound of any one of claims 1-29, or a pharmaceutically acceptable salt, solvate, hydrate, polymorph, co-crystal, tautomer, stereoisomer, isotopically labeled compound, or prodrug thereof; or
the composition of claim 30.

39. The compound of any one of claims 1-29, or a pharmaceutically acceptable salt, solvate, hydrate, polymorph, co-crystal, tautomer, stereoisomer, isotopically labeled compound, or prodrug thereof, or the composition of claim 30, for use in decreasing a ratio of an amount of a first mRNA isoform to an amount of a second mRNA isoform in a subject in need thereof or in a cell, tissue, or biological sample.

40. The compound of any one of claims 1-29, or a pharmaceutically acceptable salt, solvate, hydrate, polymorph, co-crystal, tautomer, stereoisomer, isotopically labeled compound, or prodrug thereof, or the composition of claim 30, for use in the manufacture of a medicament for decreasing a ratio of an amount of a first mRNA isoform to an amount of a second mRNA isoform in a subject in need thereof or in a cell, tissue, or biological sample.

41. The method, compound for use, pharmaceutically acceptable salt, solvate, hydrate, polymorph, co-crystal, tautomer, stereoisomer, isotopically labeled compound, or prodrug thereof for use, or composition for use of any one of claims 38-40, comprising decreasing the amount of the first mRNA isoform.

42. The method, compound for use, pharmaceutically acceptable salt, solvate, hydrate, polymorph, co-crystal, tautomer, stereoisomer, isotopically labeled compound, or prodrug thereof for use, or composition for use of any one of claims 38-41, wherein the first mRNA isoform is four-repeat Tau mRNA (4R-Tau mRNA).

43. The method, compound for use, pharmaceutically acceptable salt, solvate, hydrate, polymorph, co-crystal, tautomer, stereoisomer, isotopically labeled compound, or prodrug thereof for use, or composition for use of any one of claims 38-42, wherein the second mRNA isoform is three-repeat Tau mRNA (3R-Tau mRNA).

44. The method, compound for use, pharmaceutically acceptable salt, solvate, hydrate, polymorph, co-crystal, tautomer, stereoisomer, isotopically labeled compound, or prodrug thereof for use, or composition for use of any one of claims 38-43, further comprising decreasing a ratio of an amount of a first protein isoform to an amount of a second protein isoform.

45. The method, compound for use, pharmaceutically acceptable salt, solvate, hydrate, polymorph, co-crystal, tautomer, stereoisomer, isotopically labeled compound, or prodrug thereof for use, or composition for use of claim 44, comprising decreasing the amount of the first protein isoform.

46. The method, compound for use, pharmaceutically acceptable salt, solvate, hydrate, polymorph, co-crystal, tautomer, stereoisomer, isotopically labeled compound, or prodrug thereof for use, or composition for use of any one of claim 44 or 45, wherein the first protein isoform is four-repeat Tau (4R-Tau).

47. The method, compound for use, pharmaceutically acceptable salt, solvate, hydrate, polymorph, co-crystal, tautomer, stereoisomer, isotopically labeled compound, or prodrug thereof for use, or composition for use of any one of claims 44-46, wherein the second protein isoform is three-repeat Tau (3R-Tau).

48. A method of treating or preventing a disease in a subject in need thereof, comprising administering to the subject in need thereof an effective amount of:
a compound of any one of claims 1-29, or a pharmaceutically acceptable salt, solvate, hydrate, polymorph, co-crystal, tautomer, stereoisomer, isotopically labeled compound, or prodrug thereof; or
the composition of claim 30.

49. The compound of any one of claims 1-29, or a pharmaceutically acceptable salt, solvate, hydrate, polymorph, co-crystal, tautomer, stereoisomer, isotopically labeled compound, or prodrug thereof, or the composition of claim 30, for use in treating a disease in a subject in need thereof.

50. The compound of any one of claims 1-29, or a pharmaceutically acceptable salt, solvate, hydrate, polymorph, co-crystal, tautomer, stereoisomer, isotopically labeled compound, or prodrug thereof, or the composition of claim 30, for use in the manufacture of a medicament for treatment of a disease in a subject in need thereof.

51. The method, compound for use, pharmaceutically acceptable salt, solvate, hydrate, polymorph, co-crystal, tautomer, stereoisomer, isotopically labeled compound, or prodrug thereof for use, or composition for use of any one of claims 48-50, wherein the disease is associated with microtubule-associated protein Tau (MAPT) pre-mRNA.

52. The method, compound for use, pharmaceutically acceptable salt, solvate, hydrate, polymorph, co-crystal, tautomer, stereoisomer, isotopically labeled compound, or prodrug thereof for use, or composition for use of any one of claims 48-51, wherein the disease is a neurodegenerative disease.

53. The method, compound for use, pharmaceutically acceptable salt, solvate, hydrate, polymorph, co-crystal, tautomer, stereoisomer, isotopically labeled compound, or prodrug thereof for use, or composition for use of claim 52, wherein the neurodegenerative disease is frontotemporal dementia and parkinsonism linked to chromosome 17 (FTDP-17) or Alzheimer's disease.

54. The method, compound for use, pharmaceutically acceptable salt, solvate, hydrate, polymorph, co-crystal, tautomer, stereoisomer, isotopically labeled compound, or prodrug thereof for use, or composition for use of any one of claims 48-53, comprising mitigating pathologies relative to Tauopathy.

55. The method, compound for use, pharmaceutically acceptable salt, solvate, hydrate, polymorph, co-crystal, tautomer, stereoisomer, isotopically labeled compound, or prodrug thereof for use, or composition for use of any one of claims 48-54, comprising correcting aberrant behavior phenotypes associated with the disease.

56. A method of preparing a compound of Formula (I-a-v): 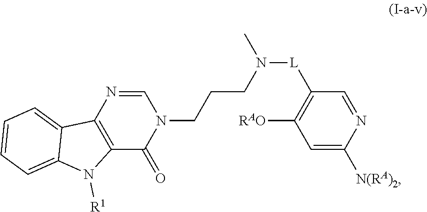
or a pharmaceutically acceptable salt, solvate, hydrate, polymorph, co-crystal, tautomer, stereoisomer, isotopically labeled compound, or prodrug thereof, comprising reacting a compound of Formula (II): 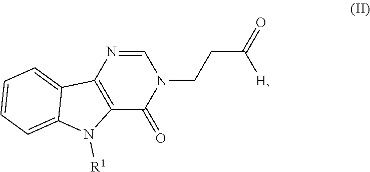
or a salt thereof, with a compound of Formula (III): 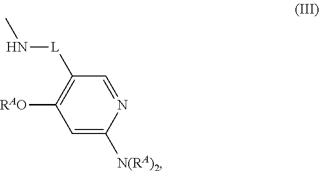
or a salt thereof, wherein:
R¹ is hydrogen or optionally substituted alkyl;
L is linker; and
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom, or two occurrences of R^{A} are joined together with their intervening atom to form an optionally substituted heterocyclic ring or optionally substituted heteroaryl ring.

57. The method of claim 56, wherein the compound of Formula (III) is of Formula (III-a): 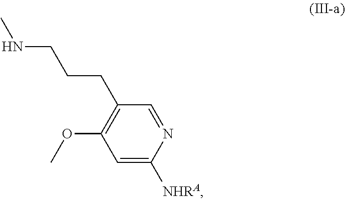
or a salt thereof, wherein R^{A} is a nitrogen protecting group.

58. The method of claim 57, further comprising deprotecting the nitrogen protecting group.

59. A kit comprising the compound of any one of claims 1-29, or a pharmaceutically acceptable salt, solvate, hydrate, polymorph, co-crystal, tautomer, stereoisomer, isotopically labeled compound, or prodrug thereof, or the composition of claim 30, and instructions for its use.


SEQUENCE LISTING (42 sequences)

SEQ ID NO: 1 - DNA, 21 bp, synthetic construct
  source 1..21: mol_type = other DNA
tgaaggtactcacactgccgc

SEQ ID NO: 2 - DNA, 18 bp, synthetic construct
  source 1..18: mol_type = other DNA
ctgtctgacgttctttgt

SEQ ID NO: 3 - RNA, 11 nt, synthetic construct
  source 1..11: mol_type = other RNA
ccggcagtgtg

SEQ ID NO: 4 - RNA, 10 nt, synthetic construct
  source 1..10: mol_type = other RNA
cacacgtcgg

SEQ ID NO: 5 - RNA, 25 nt, synthetic construct
  source 1..25: mol_type = other RNA
ggcagtgtgagtaccttcatacgtc

SEQ ID NO: 6 - RNA, 13 nt, synthetic construct
  source 1..13: mol_type = other RNA
gtccactcatgga

SEQ ID NO: 7 - DNA, 41 bp, synthetic construct
  source 1..41: mol_type = other DNA
cagacgtgtgctcttccgatctgacactccagtcccacagt

SEQ ID NO: 8 - DNA, 25 bp, synthetic construct
  source 1..25: mol_type = other DNA
nnnagatcggaagagcgtcgtgtag

SEQ ID NO: 9 - DNA, 21 bp, synthetic construct
  source 1..21: mol_type = other DNA
cagacgtgtgctcttccgatc

SEQ ID NO: 10 - DNA, 22 bp, synthetic construct
  source 1..22: mol_type = other DNA
ctacacgacgctcttccgatct

SEQ ID NO: 11 - DNA, 30 bp, synthetic construct
  source 1..30: mol_type = other DNA
gaggcgggaaggtgcagataattaataaga

SEQ ID NO: 12 - DNA, 28 bp, synthetic construct
  source 1..28: mol_type = other DNA
cagccgggaggcgggaaggtgcaaatag

SEQ ID NO: 13 - DNA, 20 bp, synthetic construct
  source 1..20: mol_type = other DNA
gccttatgcagttgctctcc

SEQ ID NO: 14 - DNA, 28 bp, synthetic construct
  source 1..28: mol_type = other DNA
gaggcgggaaggtgcagataattaataa

SEQ ID NO: 15 - DNA, 22 bp, synthetic construct
  source 1..22: mol_type = other DNA
ctggtttatgatggatgttgcc

SEQ ID NO: 16 - DNA, 24 bp, synthetic construct
  source 1..24: mol_type = other DNA
gaagaatgtcaagtccaagatcgg

SEQ ID NO: 17 - DNA, 20 bp, synthetic construct
  source 1..20: mol_type = other DNA
gactatttgcaccttcccgc

SEQ ID NO: 18 - DNA, 23 bp, synthetic construct
  source 1..23: mol_type = other DNA
ggaagtggtgtgagtgcgtacac

SEQ ID NO: 19 - DNA, 23 bp, synthetic construct
  source 1..23: mol_type = other DNA
caccttcagcccaacttccaatg

SEQ ID NO: 20 - DNA, 19 bp, synthetic construct
  source 1..19: mol_type = other DNA
agaagcaggcattggagac

SEQ ID NO: 21 - DNA, 20 bp, synthetic construct
  source 1..20: mol_type = other DNA
tcttcgttttaccatcagcc

SEQ ID NO: 22 - DNA, 18 bp, synthetic construct
  source 1..18: mol_type = other DNA
tgcccccatgttgtgatg

SEQ ID NO: 23 - DNA, 19 bp, synthetic construct
  source 1..19: mol_type = other DNA
tgtggtcatgagcccttcc

SEQ ID NO: 24 - DNA, 20 bp, synthetic construct
  source 1..20: mol_type = other DNA
cactgagaacctgaagcacc

SEQ ID NO: 25 - DNA, 21 bp, synthetic construct
  source 1..21: mol_type = other DNA
ggacgttgctaagatccagct

SEQ ID NO: 26 - DNA, 20 bp, synthetic construct
  source 1..20: mol_type = other DNA
ggcaaattcaacggcacagt

SEQ ID NO: 27 - DNA, 20 bp, synthetic construct
  source 1..20: mol_type = other DNA
gggtctcgctcctggaagat

SEQ ID NO: 28 - DNA, 21 bp, synthetic construct
  source 1..21: mol_type = other DNA
catgtacgttgctatccaggc

SEQ ID NO: 29 - DNA, 21 bp, synthetic construct
  source 1..21: mol_type = other DNA
ctccttaatgtcacgcacgat

SEQ ID NO: 30 - DNA, 20 bp, synthetic construct
  source 1..20: mol_type = other DNA
gtaacccgttgaaccccatt

SEQ ID NO: 31 - DNA, 17 bp, synthetic construct
  source 1..17: mol_type = other DNA
tccaatcggtagtagcg

SEQ ID NO: 32 - DNA, 25 bp, synthetic construct
  source 1..25: mol_type = other DNA
actttgaaccaggatggctgagccc

SEQ ID NO: 33 - DNA, 25 bp, synthetic construct
  source 1..25: mol_type = other DNA
ctgtgcatggctgtccactaacctt

SEQ ID NO: 34 - RNA, 25 nt, synthetic construct
  source 1..25: mol_type = other RNA
ggcagtgtgagtaccttcacacgtc

SEQ ID NO: 35 - RNA, 26 nt, synthetic construct
  source 1..26: mol_type = other RNA
ggcagtgtgagtaccttcacactgtc

SEQ ID NO: 36 - RNA, 26 nt, synthetic construct
  source 1..26: mol_type = other RNA
ggcagtgtgagtaccttcatactgtc

SEQ ID NO: 37 - DNA, 41 bp, synthetic construct
  source 1..41: mol_type = other DNA
tgatctacacgacgctcttccgatctgggtcccatgcgccg

SEQ ID NO: 38 - DNA, 25 bp, synthetic construct
  source 1..25: mol_type = other DNA
gatgtgctgcgagaaggctagaccc

SEQ ID NO: 39 - RNA, 35 nt, synthetic construct
  source 1..35: mol_type = other RNA
  modified_base 29: mod_base = OTHER; note = thymine
ggcagtgtgagtaccttcacacgtcccatgcgccg

SEQ ID NO: 40 - RNA, 25 nt, synthetic construct
  source 1..25: mol_type = other RNA
  modified_base 5: mod_base = OTHER; note = 7-deaza-guanosine
ggcagtgtgagtaccttcacacgtc

SEQ ID NO: 41 - RNA, 12 nt, synthetic construct
  source 1..12: mol_type = other RNA
  modified_base 1: mod_base = OTHER; note = guanosine triphosphate
gatacttacctg

SEQ ID NO: 42 - RNA, 11 nt, synthetic construct
  source 1..11: mol_type = other RNA
ggagtaagtct